SEQUENCE LISTING (238 sequences)

SEQ ID NO: 1 - protein, 8 aa, Artificial Sequence
  Description of Artificial Sequence Synthetic peptide
GFTFSNYA

SEQ ID NO: 2 - protein, 8 aa, Artificial Sequence
  Description of Artificial Sequence Synthetic peptide
ISFDGSIN

SEQ ID NO: 3 - protein, 21 aa, Artificial Sequence
  Description of Artificial Sequence Synthetic peptide
CARDRYYYDSSAYFLIDAFDI

SEQ ID NO: 4 - protein, 6 aa, Artificial Sequence
  Description of Artificial Sequence Synthetic peptide
QSVSSH

SEQ ID NO: 5 - protein, 3 aa, Artificial Sequence
  Description of Artificial Sequence Synthetic peptide
DAS

SEQ ID NO: 6 - protein, 10 aa, Artificial Sequence
  Description of Artificial Sequence Synthetic peptide
QQRSNWPPIT

SEQ ID NO: 7 - protein, 8 aa, Artificial Sequence
  Description of Artificial Sequence Synthetic peptide
GFILSDHY

SEQ ID NO: 8 - protein, 10 aa, Artificial Sequence
  Description of Artificial Sequence Synthetic peptide
SRNKAKRYTT

SEQ ID NO: 9 - protein, 18 aa, Artificial Sequence
  Description of Artificial Sequence Synthetic peptide
TRAGYYDKNGDSLDALDI

SEQ ID NO: 10 - protein, 6 aa, Artificial Sequence
  Description of Artificial Sequence Synthetic peptide
QTVSSN

SEQ ID NO: 11 - protein, 3 aa, Artificial Sequence
  Description of Artificial Sequence Synthetic peptide
GIS

SEQ ID NO: 12 - protein, 9 aa, Artificial Sequence
  Description of Artificial Sequence Synthetic peptide
QQSYSLPRT

SEQ ID NO: 13 - protein, 8 aa, Artificial Sequence
  Description of Artificial Sequence Synthetic peptide
GYSFTSHT

SEQ ID NO: 14 - protein, 8 aa, Artificial Sequence
  Description of Artificial Sequence Synthetic peptide
INTNTGNP

SEQ ID NO: 15 - protein, 25 aa, Artificial Sequence
  Description of Artificial Sequence Synthetic peptide
ARVLKCLGGSASCSGHGYYDYGMAV

SEQ ID NO: 16 - protein, 11 aa, Artificial Sequence
  Description of Artificial Sequence Synthetic peptide
QSLLHSNGYNF

SEQ ID NO: 17 - protein, 3 aa, Artificial Sequence
  Description of Artificial Sequence Synthetic peptide
LGS

SEQ ID NO: 18 - protein, 10 aa, Artificial Sequence
  Description of Artificial Sequence Synthetic peptide
MQALQTFMYT

SEQ ID NO: 19 - protein, 8 aa, Artificial Sequence
  Description of Artificial Sequence Synthetic peptide
GFTFRDYW

SEQ ID NO: 20 - protein, 8 aa, Artificial Sequence
  Description of Artificial Sequence Synthetic peptide
INRNGNEK

SEQ ID NO: 21 - protein, 19 aa, Artificial Sequence
  Description of Artificial Sequence Synthetic peptide
VRDSSPSFGPGNYYDAFDI

SEQ ID NO: 22 - protein, 6 aa, Artificial Sequence
  Description of Artificial Sequence Synthetic peptide
QDIRNE

SEQ ID NO: 23 - protein, 3 aa, Artificial Sequence
  Description of Artificial Sequence Synthetic peptide
AAS

SEQ ID NO: 24 - protein, 9 aa, Artificial Sequence
  Description of Artificial Sequence Synthetic peptide
LQDFNYPRT

SEQ ID NO: 25 - protein, 8 aa, Artificial Sequence
  Description of Artificial Sequence Synthetic peptide
GYTFSGYY

SEQ ID NO: 26 - protein, 8 aa, Artificial Sequence
  Description of Artificial Sequence Synthetic peptide
INPNSGGT

SEQ ID NO: 27 - protein, 14 aa, Artificial Sequence
  Description of Artificial Sequence Synthetic peptide
SRDPGYTYGYPLGY

SEQ ID NO: 28 - protein, 7 aa, Artificial Sequence
  Description of Artificial Sequence Synthetic peptide
QSVSSSY

SEQ ID NO: 29 - protein, 3 aa, Artificial Sequence
  Description of Artificial Sequence Synthetic peptide
GTS

SEQ ID NO: 30 - protein, 10 aa, Artificial Sequence
  Description of Artificial Sequence Synthetic peptide
QQYGNSPPYT

SEQ ID NO: 31 - protein, 8 aa, Artificial Sequence
  Description of Artificial Sequence Synthetic peptide
GFTIIDSY

SEQ ID NO: 32 - protein, 8 aa, Artificial Sequence
  Description of Artificial Sequence Synthetic peptide
INPSGSVI

SEQ ID NO: 33 - protein, 13 aa, Artificial Sequence
  Description of Artificial Sequence Synthetic peptide
ARGIYDQSDAFDL

SEQ ID NO: 34 - protein, 6 aa, Artificial Sequence
  Description of Artificial Sequence Synthetic peptide
QGISNS

SEQ ID NO: 35 - protein, 3 aa, Artificial Sequence
  Description of Artificial Sequence Synthetic peptide
GAS

SEQ ID NO: 36 - protein, 10 aa, Artificial Sequence
  Description of Artificial Sequence Synthetic peptide
QQYYITPPMT

SEQ ID NO: 37 - protein, 8 aa, Artificial Sequence
  Description of Artificial Sequence Synthetic peptide
GSTFISHA

SEQ ID NO: 38 - protein, 8 aa, Artificial Sequence
  Description of Artificial Sequence Synthetic peptide
LIPVSGTP

SEQ ID NO: 39 - protein, 17 aa, Artificial Sequence
  Description of Artificial Sequence Synthetic peptide
ATWDADSTTLLYYFMDV

SEQ ID NO: 40 - protein, 6 aa, Artificial Sequence
  Description of Artificial Sequence Synthetic peptide
QSISRW

SEQ ID NO: 41 - protein, 3 aa, Artificial Sequence
  Description of Artificial Sequence Synthetic peptide
KAS

SEQ ID NO: 42 - protein, 10 aa, Artificial Sequence
  Description of Artificial Sequence Synthetic peptide
QQYNSYSTWT

SEQ ID NO: 43 - protein, 8 aa, Artificial Sequence
  Description of Artificial Sequence Synthetic peptide
GFTFSSFA

SEQ ID NO: 44 - protein, 8 aa, Artificial Sequence
  Description of Artificial Sequence Synthetic peptide
ISYEGKNK

SEQ ID NO: 45 - protein, 14 aa, Artificial Sequence
  Description of Artificial Sequence Synthetic peptide
ARPFSMSWFEGFEF

SEQ ID NO: 46 - protein, 6 aa, Artificial Sequence
  Description of Artificial Sequence Synthetic peptide
QNINSF

SEQ ID NO: 47 - protein, 3 aa, Artificial Sequence
  Description of Artificial Sequence Synthetic peptide
EAS

SEQ ID NO: 48 - protein, 9 aa, Artificial Sequence
  Description of Artificial Sequence Synthetic peptide
QQSYTAPLT

SEQ ID NO: 49 - protein, 8 aa, Artificial Sequence
  Description of Artificial Sequence Synthetic peptide
GYTFTNYY

SEQ ID NO: 50 - protein, 8 aa, Artificial Sequence
  Description of Artificial Sequence Synthetic peptide
IYPSGGDT

SEQ ID NO: 51 - protein, 17 aa, Artificial Sequence
  Description of Artificial Sequence Synthetic peptide
ARDHLNRDSSSRGFMDY

SEQ ID NO: 52 - protein, 6 aa, Artificial Sequence
  Description of Artificial Sequence Synthetic peptide
QSISHY

SEQ ID NO: 53 - protein, 3 aa, Artificial Sequence
  Description of Artificial Sequence Synthetic peptide
DAS

SEQ ID NO: 54 - protein, 9 aa, Artificial Sequence
  Description of Artificial Sequence Synthetic peptide
QQRGTWPPS

SEQ ID NO: 55 - protein, 8 aa, Artificial Sequence
  Description of Artificial Sequence Synthetic peptide
GFNFNIFP

SEQ ID NO: 56 - protein, 8 aa, Artificial Sequence
  Description of Artificial Sequence Synthetic peptide
ISDDVTKK

SEQ ID NO: 57 - protein, 20 aa, Artificial Sequence
  Description of Artificial Sequence Synthetic peptide
ARASGWQRTGTKYYYYGMDV

SEQ ID NO: 58 - protein, 6 aa, Artificial Sequence
  Description of Artificial Sequence Synthetic peptide
QDISNN

SEQ ID NO: 59 - protein, 3 aa, Artificial Sequence
  Description of Artificial Sequence Synthetic peptide
DAS

SEQ ID NO: 60 - protein, 9 aa, Artificial Sequence
  Description of Artificial Sequence Synthetic peptide
LQYDNLPYS

SEQ ID NO: 61 - protein, 8 aa, Artificial Sequence
  Description of Artificial Sequence Synthetic peptide
GFTFSDYY

SEQ ID NO: 62 - protein, 8 aa, Artificial Sequence
  Description of Artificial Sequence Synthetic peptide
ISTSGSTM

SEQ ID NO: 63 - protein, 13 aa, Artificial Sequence
  Description of Artificial Sequence Synthetic peptide
ARGIYYQSDAFDI

SEQ ID NO: 64 - protein, 6 aa, Artificial Sequence
  Description of Artificial Sequence Synthetic peptide
QGISNS

SEQ ID NO: 65 - protein, 3 aa, Artificial Sequence
  Description of Artificial Sequence Synthetic peptide
AAS

SEQ ID NO: 66 - protein, 10 aa, Artificial Sequence
  Description of Artificial Sequence Synthetic peptide
QQYYSTPPMT

SEQ ID NO: 67 - protein, 8 aa, Artificial Sequence
  Description of Artificial Sequence Synthetic peptide
GYTFTSSY

SEQ ID NO: 68 - protein, 8 aa, Artificial Sequence
  Description of Artificial Sequence Synthetic peptide
IYPSGGNT

SEQ ID NO: 69 - protein, 17 aa, Artificial Sequence
  Description of Artificial Sequence Synthetic peptide
ARDHLNRDSTSRGFIDS

SEQ ID NO: 70 - protein, 6 aa, Artificial Sequence
  Description of Artificial Sequence Synthetic peptide
QSVGNY

SEQ ID NO: 71 - protein, 3 aa, Artificial Sequence
  Description of Artificial Sequence Synthetic peptide
DAS

SEQ ID NO: 72 - protein, 9 aa, Artificial Sequence
  Description of Artificial Sequence Synthetic peptide
EQRGDWPLT

SEQ ID NO: 73 - protein, 8 aa, Artificial Sequence
  Description of Artificial Sequence Synthetic peptide
GYTFTNYP

SEQ ID NO: 74 - protein, 8 aa, Artificial Sequence
  Description of Artificial Sequence Synthetic peptide
INTNTGKP

SEQ ID NO: 75 - protein, 16 aa, Artificial Sequence
  Description of Artificial Sequence Synthetic peptide
ARGRGATTVTTYYFDY

SEQ ID NO: 76 - protein, 6 aa, Artificial Sequence
  Description of Artificial Sequence Synthetic peptide
QSVSSN

SEQ ID NO: 77 - protein, 3 aa, Artificial Sequence
  Description of Artificial Sequence Synthetic peptide
GAS

SEQ ID NO: 78 - protein, 10 aa, Artificial Sequence
  Description of Artificial Sequence Synthetic peptide
QHYINRPGRT

SEQ ID NO: 79 - protein, 8 aa, Artificial Sequence
  Description of Artificial Sequence Synthetic peptide
GYTFIAFY

SEQ ID NO: 80 - protein, 8 aa, Artificial Sequence
  Description of Artificial Sequence Synthetic peptide
INPYSGDT

SEQ ID NO: 81 - protein, 24 aa, Artificial Sequence
  Description of Artificial Sequence Synthetic peptide
ARTVYVDKGMVMVRRLYQYFGMDV

SEQ ID NO: 82 - protein, 7 aa, Artificial Sequence
  Description of Artificial Sequence Synthetic peptide
QTVSSSY

SEQ ID NO: 83 - protein, 3 aa, Artificial Sequence
  Description of Artificial Sequence Synthetic peptide
GAS

SEQ ID NO: 84 - protein, 10 aa, Artificial Sequence
  Description of Artificial Sequence Synthetic peptide
QQYGISPEFT

SEQ ID NO: 85 - protein, 8 aa, Artificial Sequence
  Description of Artificial Sequence Synthetic peptide
GFTFSDYF

SEQ ID NO: 86 - protein, 8 aa, Artificial Sequence
  Description of Artificial Sequence Synthetic peptide
ISDNGNTI

SEQ ID NO: 87 - protein, 13 aa, Artificial Sequence
  Description of Artificial Sequence Synthetic peptide
ARGLYIQSDAFDL

SEQ ID NO: 88 - protein, 6 aa, Artificial Sequence
  Description of Artificial Sequence Synthetic peptide
QGLSNS

SEQ ID NO: 89 - protein, 3 aa, Artificial Sequence
  Description of Artificial Sequence Synthetic peptide
AAS

SEQ ID NO: 90 - protein, 10 aa, Artificial Sequence
  Description of Artificial Sequence Synthetic peptide
QQYYNTPPIT

SEQ ID NO: 91 - protein, 8 aa, Artificial Sequence
  Description of Artificial Sequence Synthetic peptide
GYNFTNYW

SEQ ID NO: 92 - protein, 8 aa, Artificial Sequence
  Description of Artificial Sequence Synthetic peptide
IYPGDSDS

SEQ ID NO: 93 - protein, 27 aa, Artificial Sequence
  Description of Artificial Sequence Synthetic peptide
ARRPREQLGRLLLGDVVPHGRNDAFDI

SEQ ID NO: 94 - protein, 6 aa, Artificial Sequence
  Description of Artificial Sequence Synthetic peptide
QSISTY

SEQ ID NO: 95 - protein, 3 aa, Artificial Sequence
  Description of Artificial Sequence Synthetic peptide
SAS

SEQ ID NO: 96 - protein, 9 aa, Artificial Sequence
  Description of Artificial Sequence Synthetic peptide
QQSYGTLWT

SEQ ID NO: 97 - protein, 8 aa, Artificial Sequence
  Description of Artificial Sequence Synthetic peptide
GFIFKTYG

SEQ ID NO: 98 - protein, 8 aa, Artificial Sequence
  Description of Artificial Sequence Synthetic peptide
IWYDGSNE

SEQ ID NO: 99 - protein, 18 aa, Artificial Sequence
  Description of Artificial Sequence Synthetic peptide
ARDEAVGPYQYAAEYFHH

SEQ ID NO: 100 - protein, 6 aa, Artificial Sequence
  Description of Artificial Sequence Synthetic peptide
KSVTSN

SEQ ID NO: 101 - protein, 3 aa, Artificial Sequence
  Description of Artificial Sequence Synthetic peptide
GAS

SEQ ID NO: 102 - protein, 8 aa, Artificial Sequence
  Description of Artificial Sequence Synthetic peptide
QQYNNWLT

SEQ ID NO: 103 - protein, 8 aa, Artificial Sequence
  Description of Artificial Sequence Synthetic peptide
GFTFSSYA

SEQ ID NO: 104 - protein, 8 aa, Artificial Sequence
  Description of Artificial Sequence Synthetic peptide
ISPSGSTI

SEQ ID NO: 105 - protein, 13 aa, Artificial Sequence
  Description of Artificial Sequence Synthetic peptide
VRGVYVQSDAFDI

SEQ ID NO: 106 - protein, 6 aa, Artificial Sequence
  Description of Artificial Sequence Synthetic peptide
QGISYS

SEQ ID NO: 107 - protein, 3 aa, Artificial Sequence
  Description of Artificial Sequence Synthetic peptide
AAS

SEQ ID NO: 108 - protein, 10 aa, Artificial Sequence
  Description of Artificial Sequence Synthetic peptide
QQYYSTPPIT

SEQ ID NO: 109 - protein, 8 aa, Artificial Sequence
  Description of Artificial Sequence Synthetic peptide
GVTFSDYD

SEQ ID NO: 110 - protein, 8 aa, Artificial Sequence
  Description of Artificial Sequence Synthetic peptide
IRSSGGTT

SEQ ID NO: 111 - protein, 10 aa, Artificial Sequence
  Description of Artificial Sequence Synthetic peptide
VRDKDGVFDY

SEQ ID NO: 112 - protein, 6 aa, Artificial Sequence
  Description of Artificial Sequence Synthetic peptide
QDISSW

SEQ ID NO: 113 - protein, 3 aa, Artificial Sequence
  Description of Artificial Sequence Synthetic peptide
KAS

SEQ ID NO: 114 - protein, 10 aa, Artificial Sequence
  Description of Artificial Sequence Synthetic peptide
QQYNTYPHST

SEQ ID NO: 115 - protein, 8 aa, Artificial Sequence
  Description of Artificial Sequence Synthetic peptide
GFTFSDYY

SEQ ID NO: 116 - protein, 8 aa, Artificial Sequence
  Description of Artificial Sequence Synthetic peptide
ISISGSTI

SEQ ID NO: 117 - protein, 13 aa, Artificial Sequence
  Description of Artificial Sequence Synthetic peptide
ARGIYHQSDAFDI

SEQ ID NO: 118 - protein, 6 aa, Artificial Sequence
  Description of Artificial Sequence Synthetic peptide
QGISNS

SEQ ID NO: 119 - protein, 3 aa, Artificial Sequence
  Description of Artificial Sequence Synthetic peptide
AAS

SEQ ID NO: 120 - protein, 10 aa, Artificial Sequence
  Description of Artificial Sequence Synthetic peptide
QQYYSTPPIT

SEQ ID NO: 121 - protein, 8 aa, Artificial Sequence
  Description of Artificial Sequence Synthetic peptide
GFTFRDYW

SEQ ID NO: 122 - protein, 8 aa, Artificial Sequence
  Description of Artificial Sequence Synthetic peptide
INRNGNEK

SEQ ID NO: 123 - protein, 19 aa, Artificial Sequence
  Description of Artificial Sequence Synthetic peptide
VRDNSPSFGPGNYYDAFDI

SEQ ID NO: 124 - protein, 6 aa, Artificial Sequence
  Description of Artificial Sequence Synthetic peptide
QDIRNE

SEQ ID NO: 125 - protein, 3 aa, Artificial Sequence
  Description of Artificial Sequence Synthetic peptide
AAS

SEQ ID NO: 126 - protein, 9 aa, Artificial Sequence
  Description of Artificial Sequence Synthetic peptide
LQDYNYPRT

SEQ ID NO: 127 - protein, 8 aa, Artificial Sequence
  Description of Artificial Sequence Synthetic peptide
GYTFTDYY

SEQ ID NO: 128 - protein, 8 aa, Artificial Sequence
  Description of Artificial Sequence Synthetic peptide
ISPKSGGT

SEQ ID NO: 129 - protein, 19 aa, Artificial Sequence
  Description of Artificial Sequence Synthetic peptide
TRDNYNSWRGPDFYTGVDV

SEQ ID NO: 130 - protein, 6 aa, Artificial Sequence
  Description of Artificial Sequence Synthetic peptide
QSVSSY

SEQ ID NO: 131 - protein, 3 aa, Artificial Sequence
  Description of Artificial Sequence Synthetic peptide
NAS

SEQ ID NO: 132 - protein, 9 aa, Artificial Sequence
  Description of Artificial Sequence Synthetic peptide
QQRSSLGLS

SEQ ID NO: 133 - protein, 8 aa, Artificial Sequence
  Description of Artificial Sequence Synthetic peptide
GYSLTRYY

SEQ ID NO: 134 - protein, 8 aa, Artificial Sequence
  Description of Artificial Sequence Synthetic peptide
ISPSGGGT

SEQ ID NO: 135 - protein, 16 aa, Artificial Sequence
  Description of Artificial Sequence Synthetic peptide
ARDACSGGSCYTPFDY

SEQ ID NO: 136 - protein, 6 aa, Artificial Sequence
  Description of Artificial Sequence Synthetic peptide
QSVSSN

SEQ ID NO: 137 - protein, 3 aa, Artificial Sequence
  Description of Artificial Sequence Synthetic peptide
GAS

SEQ ID NO: 138 - protein, 9 aa, Artificial Sequence
  Description of Artificial Sequence Synthetic peptide
QQYNNWPRT

SEQ ID NO: 139 - protein, 8 aa, Artificial Sequence
  Description of Artificial Sequence Synthetic peptide
GFTFSSYG

SEQ ID NO: 140 - protein, 8 aa, Artificial Sequence
  Description of Artificial Sequence Synthetic peptide
IWLDGTNK

SEQ ID NO: 141 - protein, 19 aa, Artificial Sequence
  Description of Artificial Sequence Synthetic peptide
ARRGFHYDSSGYYYYGMDV

SEQ ID NO: 142 - protein, 11 aa, Artificial Sequence
  Description of Artificial Sequence Synthetic peptide
QSLLHSNGYNY

SEQ ID NO: 143 - protein, 3 aa, Artificial Sequence
  Description of Artificial Sequence Synthetic peptide
LGS

SEQ ID NO: 144 - protein, 10 aa, Artificial Sequence
  Description of Artificial Sequence Synthetic peptide
MQALQTPPFT

SEQ ID NO: 145 - protein, 8 aa, Artificial Sequence
  Description of Artificial Sequence Synthetic peptide
GFTFSNYD

SEQ ID NO: 146 - protein, 7 aa, Artificial Sequence
  Description of Artificial Sequence Synthetic peptide
IDTSGNT

SEQ ID NO: 147 - protein, 16 aa, Artificial Sequence
  Description of Artificial Sequence Synthetic peptide
VRLGGYIGNDRDAFDI

SEQ ID NO: 148 - protein, 6 aa, Artificial Sequence
  Description of Artificial Sequence Synthetic peptide
QDISSW

SEQ ID NO: 149 - protein, 3 aa, Artificial Sequence
  Description of Artificial Sequence Synthetic peptide
KAS

SEQ ID NO: 150 - protein, 10 aa, Artificial Sequence
  Description of Artificial Sequence Synthetic peptide
QQYNTYPHST

SEQ ID NO: 151 - protein, 8 aa, Artificial Sequence
  Description of Artificial Sequence Synthetic peptide
GFIFGDFA

SEQ ID NO: 152 - protein, 10 aa, Artificial Sequence
  Description of Artificial Sequence Synthetic peptide
IRSQAHGGTT

SEQ ID NO: 153 - protein, 18 aa, Artificial Sequence
  Description of Artificial Sequence Synthetic peptide
TREGVVVAARYYYYIMDV

SEQ ID NO: 154 - protein, 6 aa, Artificial Sequence
  Description of Artificial Sequence Synthetic peptide
HNISRY

SEQ ID NO: 155 - protein, 3 aa, Artificial Sequence
  Description of Artificial Sequence Synthetic peptide
AAS

SEQ ID NO: 156 - protein, 9 aa, Artificial Sequence
  Description of Artificial Sequence Synthetic peptide
QQNYRTPRT

SEQ ID NO: 157 - protein, 8 aa, Artificial Sequence
  Description of Artificial Sequence Synthetic peptide
GGIFSNYA

SEQ ID NO: 158 - protein, 8 aa, Artificial Sequence
  Description of Artificial Sequence Synthetic peptide
FIPIVNIG

SEQ ID NO: 159 - protein, 17 aa, Artificial Sequence
  Description of Artificial Sequence Synthetic peptide
ARDLEAANSVILPRLFY

SEQ ID NO: 160 - protein, 6 aa, Artificial Sequence
  Description of Artificial Sequence Synthetic peptide
QGISNS

SEQ ID NO: 161 - protein, 3 aa, Artificial Sequence
  Description of Artificial Sequence Synthetic peptide
AAS

SEQ ID NO: 162 - protein, 10 aa, Artificial Sequence
  Description of Artificial Sequence Synthetic peptide
QQYYSTPPIT

SEQ ID NO: 163 - protein, 8 aa, Artificial Sequence
  Description of Artificial Sequence Synthetic peptide
GFTFTDYY

SEQ ID NO: 164 - protein, 8 aa, Artificial Sequence
  Description of Artificial Sequence Synthetic peptide
ISPSGSTI

SEQ ID NO: 165 - protein, 13 aa, Artificial Sequence
  Description of Artificial Sequence Synthetic peptide
ARGIYYQSDAFDT

SEQ ID NO: 166 - protein, 6 aa, Artificial Sequence
  Description of Artificial Sequence Synthetic peptide
QVIRNS

SEQ ID NO: 167 - protein, 3 aa, Artificial Sequence
  Description of Artificial Sequence Synthetic peptide
AAS

SEQ ID NO: 168 - protein, 10 aa, Artificial Sequence
  Description of Artificial Sequence Synthetic peptide
QQYYSTPPIT

SEQ ID NO: 169 - protein, 8 aa, Artificial Sequence
  Description of Artificial Sequence Synthetic peptide
GYTFIDYF

SEQ ID NO: 170 - protein, 8 aa, Artificial Sequence
  Description of Artificial Sequence Synthetic peptide
IYPKSGET

SEQ ID NO: 171 - protein, 14 aa, Artificial Sequence
  Description of Artificial Sequence Synthetic peptide
ARDIAPTGAWWFDS

SEQ ID NO: 172 - protein, 7 aa, Artificial Sequence
  Description of Artificial Sequence Synthetic peptide
QMLSSSR

SEQ ID NO: 173 - protein, 3 aa, Artificial Sequence
  Description of Artificial Sequence Synthetic peptide
GAS

SEQ ID NO: 174 - protein, 8 aa, Artificial Sequence
  Description of Artificial Sequence Synthetic peptide
QQYGSPRT

SEQ ID NO: 175 - protein, 8 aa, Artificial Sequence
  Description of Artificial Sequence Synthetic peptide
GFTFSSHA

SEQ ID NO: 176 - protein, 8 aa, Artificial Sequence
  Description of Artificial Sequence Synthetic peptide
ISYDGSNK

SEQ ID NO: 177 - protein, 10 aa, Artificial Sequence
  Description of Artificial Sequence Synthetic peptide
VRWVAYYFDN

SEQ ID NO: 178 - protein, 7 aa, Artificial Sequence
  Description of Artificial Sequence Synthetic peptide
QSVSSSS

SEQ ID NO: 179 - protein, 3 aa, Artificial Sequence
  Description of Artificial Sequence Synthetic peptide
GTS

SEQ ID NO: 180 - protein, 10 aa, Artificial Sequence
  Description of Artificial Sequence Synthetic peptide
QYYGSLPPIT

SEQ ID NO: 181 - protein, 8 aa, Artificial Sequence
  Description of Artificial Sequence Synthetic peptide
GDSISSYY

SEQ ID NO: 182 - protein, 7 aa, Artificial Sequence
  Description of Artificial Sequence Synthetic peptide
ISYTGST

SEQ ID NO: 183 - protein, 14 aa, Artificial Sequence
  Description of Artificial Sequence Synthetic peptide
ARLGYSHPYWYFDL

SEQ ID NO: 184 - protein, 6 aa, Artificial Sequence
  Description of Artificial Sequence Synthetic peptide
QSISNF

SEQ ID NO: 185 - protein, 3 aa, Artificial Sequence
  Description of Artificial Sequence Synthetic peptide
AAS

SEQ ID NO: 186 - protein, 10 aa, Artificial Sequence
  Description of Artificial Sequence Synthetic peptide
QQSYSPPLIT

SEQ ID NO: 187 - protein, 8 aa, Artificial Sequence
  Description of Artificial Sequence Synthetic peptide
GFTFSDYY

SEQ ID NO: 188 - protein, 8 aa, Artificial Sequence
  Description of Artificial Sequence Synthetic peptide
ISGSGKIT

SEQ ID NO: 189 - protein, 15 aa, Artificial Sequence
  Description of Artificial Sequence Synthetic peptide
ARVQGEQWRGLHFDS

SEQ ID NO: 190 - protein, 6 aa, Artificial Sequence
  Description of Artificial Sequence Synthetic peptide
QDISNY

SEQ ID NO: 191 - protein, 3 aa, Artificial Sequence
  Description of Artificial Sequence Synthetic peptide
DAS

SEQ ID NO: 192 - protein, 8 aa, Artificial Sequence
  Description of Artificial Sequence Synthetic peptide
QHRSNWPA

SEQ ID NO: 193 - protein, 8 aa, Artificial Sequence
  Description of Artificial Sequence Synthetic peptide
GFRFGDYA

SEQ ID NO: 194 - protein, 8 aa, Artificial Sequence
  Description of Artificial Sequence Synthetic peptide
INWDSGDI

SEQ ID NO: 195 - protein, 23 aa, Artificial Sequence
  Description of Artificial Sequence Synthetic peptide
AKDSGWLRRGDYDTSGFYGPIDY

SEQ ID NO: 196 - protein, 6 aa, Artificial Sequence
  Description of Artificial Sequence Synthetic peptide
QYISTY

SEQ ID NO: 197 - protein, 3 aa, Artificial Sequence
  Description of Artificial Sequence Synthetic peptide
SAS

SEQ ID NO: 198 - protein, 9 aa, Artificial Sequence
  Description of Artificial Sequence Synthetic peptide
QQSYGTLLT

SEQ ID NO: 199 - protein, 128 aa, Artificial Sequence
  Description of Artificial Sequence Synthetic polypeptide
EVQLVESGGGVVQPGRSLRLSCAASGFTFSNYALHWVRQAPGKGLEWVAVISFDGSINKYYADSVKGRFTISRDNSKNTLYLQMNSLRADGTAVYYCARDRYYYDSSAYFLIDAFDIWGQGTTVTVSS

SEQ ID NO: 200 - protein, 108 aa, Artificial Sequence
  Description of Artificial Sequence Synthetic polypeptide
EIVLTQSPATLSLSPGERATLSCRASQSVSSHLAWYQQKPGQAPRLLIYDASNRATGIPARFSGSGSGTDFTLTISSLEPEDFAIYYCQQRSNWPPITFGGGTKLEIK

SEQ ID NO: 201 - protein, 127 aa, Artificial Sequence
  Description of Artificial Sequence Synthetic polypeptide
EVQLVESGGGLVQPGGSLSLACEVSGFILSDHYIDWVRQAPGKGLEWVGRSRNKAKRYTTEYAASVKGRFTISRDDSNNSLYLLMKSLKTEDTAVYFCTRAGYYDKNGDSLDALDIWGQGTMVTVSS

SEQ ID NO: 202 - protein, 107 aa, Artificial Sequence
  Description of Artificial Sequence Synthetic polypeptide
DIQVTQSPSSLSASVGDRVTITCRTSQTVSSNLNWYQQRPGKAPKLLISGISDLHSGVPSRFSGSGSGTDSTLTISSLQPEDSATYYCQQSYSLPRTFGQGTKVEIK

SEQ ID NO: 203 - protein, 132 aa, Artificial Sequence
  Description of Artificial Sequence Synthetic polypeptide
EVQLVESGSELKKPGASVRVSCEASGYSFTSHTIVWMRQAPGQGLECLGWINTNTGNPTYAQGFTGRFVFSLDTSVSTAYLQISSLKAADTAVYYCARVLKCLGGSASCSGHGYYDYGMAVWGQGTTVTVSS

SEQ ID NO: 204 - protein, 113 aa, Artificial Sequence
  Description of Artificial Sequence Synthetic polypeptide
DIVLTQSPLSLPVTPGEPASISCRSSQSLLHSNGYNFLHWYLQKPGQSPQLLIYLGSIRASGVPDRFSGSGSGTDFTLKISRVEAEDVGVYYCMQALQTFMYTFGQGTKLEIK

SEQ ID NO: 205 - protein, 126 aa, Artificial Sequence
  Description of Artificial Sequence Synthetic polypeptide
EVQLVESGGGLVQPGGSLRLSCAASGFTFRDYWMIWVRQTPGKGLEWVANINRNGNEKHYVDSLKGRFTISRDNTKNSLYLQVNGLSAEDTAVYYCVRDSSPSFGPGNYYDAFDIWGQGTTVTVSS

SEQ ID NO: 206 - protein, 107 aa, Artificial Sequence
  Description of Artificial Sequence Synthetic polypeptide
DIRVTQSPSSLSASVGDRVTITCRASQDIRNEVGWYQQKPGQAPKVLIFAASTLQSGVPSRFRGSGSGTVFTLTISSLQPEDLATYYCLQDFNYPRTFGQGTKVDIK

SEQ ID NO: 207 - protein, 121 aa, Artificial Sequence
  Description of Artificial Sequence Synthetic polypeptide
QVQLVQSGAEVKKPGAAVKVSCKASGYTFSGYYIHWVRQAPGQGLEWMGWINPNSGGTDFAQKFQGRVTMTRDTSISTAYMELSRLRSDDTAVYLCSRDPGYTYGYPLGYWGQGTLVTVSS

SEQ ID NO: 208 - protein, 109 aa, Artificial Sequence
  Description of Artificial Sequence Synthetic polypeptide
ETTLTQSPGTLSLSPGERATLSCRASQSVSSSYLAWYQQKPGQAPRLLIYGTSSRATGIPDRFSGRGSGTDFTLTISRLEPEDFAVYYCQQYGNSPPYTFGQGTKVDIK

SEQ ID NO: 209 - protein, 120 aa, Artificial Sequence
  Description of Artificial Sequence Synthetic polypeptide
QVQLVQSGGRLVKPGGSLRLSCAASGFTIIDSYMSWIRQAPGKGLEWISYINPSGSVIYYSDSVKGRFISSRDNAKNSVYLQLSSLRAEDAAVYYCARGIYDQSDAFDLWGQGTTVTVSS

SEQ ID NO: 210 - protein, 108 aa, Artificial Sequence
  Description of Artificial Sequence Synthetic polypeptide
DIVLTQSPSSLSASVGDRVTITCRASQGISNSLAWYQQRPGKAPKLLIYGASRLGSGVPSRFSGSGSGADYTLTISGLQPEDFATYYCQQYYITPPMTFGPGTKVDIK

SEQ ID NO: 211 - protein, 124 aa, Artificial Sequence
  Description of Artificial Sequence Synthetic polypeptide
QVQLVQSGAEVKKSGSSVTVSCKASGSTFISHAISWVRQAPGEGLEWMGRLIPVSGTPKYAHKFQGRLTITADDSTTTVYLTLRSLRFEDTAVYYCATWDADSTTLLYYFMDVWGKGTTVTVSS

SEQ ID NO: 212 - protein, 108 aa, Artificial Sequence
  Description of Artificial Sequence Synthetic polypeptide
DIQVTQSPSTLSASVGDRVTITCRASQSISRWLAWYQQKPGKAPKLLIYKASSLESGVPSRFSGTGSGTEFTLTISSLQPDDFATYYCQQYNSYSTWTFGQGTKLEIK

SEQ ID NO: 213 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGRPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 214 - protein, 20 aa, Artificial Sequence
  Description of Artificial Sequence Synthetic peptide
ARDRYYYDSSAYFLIDAFDI

SEQ ID NO: 215 - protein, 16 aa, Artificial Sequence
  Description of Artificial Sequence Synthetic peptide
GGGSGGGSGGGSGGGS

SEQ ID NO: 216 - protein, 96 aa, Artificial Sequence
  Description of Artificial Sequence Synthetic polypeptide
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYC

SEQ ID NO: 217 - protein, 109 aa, Artificial Sequence
  Description of Artificial Sequence Synthetic polypeptide
EVQLVESGGGLVKSGGSLRLSCAASGFTFSDYYMSWIRQAPGKGLEWVSYISTSGSTMYYADSVKGRFTISRDNAKNLLYLQMNSLRAEDTAVYYCARGIYYQSDAFDI

SEQ ID NO: 218 - protein, 109 aa, Artificial Sequence
  Description of Artificial Sequence Synthetic polypeptide
EVQLVESGGNLVKPGGSLRLSCAASGFTFSDYYMSWIRQAPGKGLDWVSYISISGSTIYYADSVKGRFTASRDNAKNSLYLQMNSLRAEDTAVYYCARGIYHQSDAFDI

SEQ ID NO: 219 - protein, 109 aa, Artificial Sequence
  Description of Artificial Sequence Synthetic polypeptide
QVQLVESGGGLVKPGGSLRLSCEASGFTFSDYFVSWIRQAPGKGLQWISYISDNGNTINYADSVKGRFTFSRDNAKNSVYLQMKSLRAEDTAVYYCARGLYIQSDAFDL

SEQ ID NO: 220 - protein, 109 aa, Artificial Sequence
  Description of Artificial Sequence Synthetic polypeptide
EVQLVESGGGLVKPGGSLRLSCAVSGFTFTDYYMSWIRQAPGKGLEWISYISPSGSTINYAESVKGRFTFSRDNAKNSVYLQMDGLRTEDTAVYYCARGIYYQSDAFDT

SEQ ID NO: 221 - protein, 88 aa, Artificial Sequence
  Description of Artificial Sequence Synthetic polypeptide
DIQMTQSPSSLSASVGDRVTITCRASQGISNSLAWYQQKPGKAPKLLLYAASRLESGVPSRFSGSGSGTDYTLTISSLQPEDFATYYC

SEQ ID NO: 222 - protein, 98 aa, Artificial Sequence
  Description of Artificial Sequence Synthetic polypeptide
DIQLTQSPSSLSASVGDRATITCRASQGISNSLAWYQQKPGKAPKLLLYAASRLESGVPSRFSGSGSGTDYSLTISSLQPEDFATYYCQQYYSTPPMT

SEQ ID NO: 223 - protein, 98 aa, Artificial Sequence
  Description of Artificial Sequence Synthetic polypeptide
DIRLTQSPSSLSASVGDRVTITCRASQGISNSLAWYQQKPGKAPKLLLYAASRLESGVPSRFSGSGSGTDYTLTISSLQPEDFATYYCQQYYSTPPIT

SEQ ID NO: 224 - protein, 98 aa, Artificial Sequence
  Description of Artificial Sequence Synthetic polypeptide
DIRMTQSPSSLSASVGDRVTITCRASQGLSNSLAWYQQKPGKAPKLLLYAASRLGSGVPSRFSGSGSGTDYTLTISSLQPEDFATYYCQQYYNTPPIT

SEQ ID NO: 225 - protein, 98 aa, Artificial Sequence
  Description of Artificial Sequence Synthetic polypeptide
DIVMTQTPSSLSASVGDRVTITCRASQVIRNSLAWYQQKPGKAPKLLLYAASRLGSGVPSRFSGNGSGAEYTLTISSLQPEDFATYYCQQYYSTPPIT

SEQ ID NO: 226 - protein, 59 aa, Chikungunya virus
PDTPDRTLMSQQSGNVKITVNSQTVRYKCNCGDSNEGLTTTDKVINNCKVDQCHAAVTN

SEQ ID NO: 227 - protein, 59 aa, Chikungunya virus
PDTPDRTLMSQQSGNVKITVNGQTVRYKCNCGGSNEGLTTTDKVINNCKVDQCHAAVTN

SEQ ID NO: 228 - protein, 59 aa, Onyong-nyong virus
PDTPDYTLMTQQAGNVKITVDGQTVRYKCKCDGSNEGLITTDKVINNCKVDQCHTAVTN

SEQ ID NO: 229 - protein, 59 aa, Mayaro virus
PDVPDRTILSQQSGNVKITVNGRTVKYSCSCGSKPSGTTTTDKTINSCTVDKCQAYVTS

SEQ ID NO: 230 - protein, 59 aa, Mayaro virus
PDIPDRTILSQQSGNVKITVNGRTVKYSCSCGSKPSGTTTTDKTINSCTVDKCQAYVTS

SEQ ID NO: 231 - protein, 59 aa, Mayaro virus
PDIPDRTILSQQSGNVKITVNGRTVRYSCSCGSKPSGTTTTDKTINSCTVDKCQAYVTS

SEQ ID NO: 232 - protein, 59 aa, Ross River virus
PDIPDRTLLSQTAGNVKITAGGRTIRYNCTCGRDNVGTTSTDKTINTCKIDQCHAAVTS

SEQ ID NO: 233 - protein, 59 aa, Semliki Forest virus
PDTPDRTLLSQQSGNVKITVGGKKVKYNCTCGTGNVGTTNSDMTINTCLIEQCHVSVTD

SEQ ID NO: 234 - protein, 61 aa, Sindbis virus
RPHAYTSYLEESSGKVYAKPPSGKNITYECKCGDYKTGTVSTRTEITGCTAIKQCVAYKSD

SEQ ID NO: 235 - protein, 96 aa, Artificial Sequence
  Description of Artificial Sequence Synthetic polypeptide
QVQLVESGGGVVQPGRSLRLSCAASGFTFSSYGMHWVRQAPGKGLEWVAVISYDGSNKYYADSVKGRFTISRDNSKNTLYLQMNSLRAEGTAVYYC

SEQ ID NO: 236 - protein, 117 aa, Artificial Sequence
  Description of Artificial Sequence Synthetic polypeptide
EVQLVESGGGVVQPGRSLRLSCAASGFTFSNYALHWVRQAPGKGLEWVAVISFDGSINKYYADSVKGRFTISRDNSKNTLYLQMNSLRADGTAVYYCARDRYYYDSSAYFLIDAFDI

SEQ ID NO: 237 - protein, 88 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYC

SEQ ID NO: 238 - protein, 98 aa, Artificial Sequence
  Description of Artificial Sequence Synthetic polypeptide
EIVLTQSPATLSLSPGERATLSCRASQSVSSHLAWYQQKPGQAPRLLIYDASNRATGIPARFSGSGSGTDFTLTISSLEPEDFAIYYCQQRSNWPPIT

(19) United States
(12) Patent Application Publication
(10) Pub. No.: US 2025/0320277 A1
(43) Pub. Date: Oct. 16, 2025

(54) ANTIBODIES OR ANTIBODY-FRAGMENTS THEREOF TARGETING ALPHAVIRUSES, AND COMPOSITIONS AND METHODS COMPRISING SAME

(71) Applicant: Albert Einstein College of Medicine, Bronx, NY (US)

(72) Inventors: Jonathan R. LAI, Dobbs Ferry, NY (US); Ryan Malonis, Bronx, NY (US); Margaret Kielian, Dobbs Ferry, NY (US)

(21) Appl. No.: 18/026,392

(22) PCT Filed: Sep. 15, 2021
(86) PCT No.: PCT/US21/50456

§ 371 Date: Mar. 15, 2023

Related U.S. Application Data
(60) Provisional application No. 63/078,614, filed on Sep. 15, 2020.
(60) Provisional application No. 63/221,159, filed on Jul. 13, 2021.

(51) Int. Cl. C07K 16/10; A61K 39/00; A61P 31/14; G01N 33/569

(52) U.S. Cl. CPC C07K 16/10; A61P 31/14; G01N 33/56983; A61K 2039/505; C07K 2317/33; C07K 2317/52; C07K 2317/565; C07K 2317/76; C07K 2317/92; G01N 2333/181

(57) ABSTRACT

Provided are high affinity anti-alphavirus antibody or alphavirus-binding fragment thereof, as well as methods of use and devices employing such antibodies and/or fragments.

### CROSS REFERENCE TO RELATED APPLICATIONS

This application is a national stage filing under 35 U.S.C. § 371 of PCT International Application No. PCT/US2021/050456, filed Sep. 15, 2021, which claims priority to U.S. Provisional Application No: 63/078,614, filed Sep. 15, 2020, and U.S. Provisional Application No: 63/221,159, filed Jul. 13, 2021; the contents of each of said applications are incorporated herein by reference in their entirety.

### STATEMENT OF GOVERNMENT SUPPORT

This invention was made with government support under grant numbers NIH R01-AI125462, R01 AI114816, U19 AI142790, AI201800001, R01 AI075647, R01-AI132633, T32-GM007288, F30-AI150055, and P30CA013330 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Nov. 16, 2021, is named AET-02025_SL.txt and is 83,985 bytes in size.

### BACKGROUND OF THE INVENTION

The disclosures of all publications, patents, patent application publications and books referred to herein, are hereby incorporated by reference in their entirety into the subject application to more fully describe the art to which the subject invention pertains.

Alphaviruses are enveloped, positive sense single-stranded RNA viruses that can cause significant human diseases ranging from arthritis to encephalitis (1-3). Alphaviruses that are associated with musculoskeletal disease (arthritogenic alphaviruses) include Chikungunya virus (CHIKV), Mayaro virus (MAYV), Ross River virus (RRV), O'nyong-nyong virus (ONNV), and others; these viruses are globally distributed and transmitted by mosquitos. Symptomatic infection by arthritogenic alphaviruses is characterized by fever, rash, myalgia, as well as both acute and chronic peripheral polyarthralgia (4, 5). The arthropathy can be debilitating and persist for months to years after infection. More severe manifestations of alphavirus disease—including encephalopathy and mortality—have been reported (6, 7). These viruses cause endemic disease as well as large, sporadic global outbreaks (8-10). Currently, there are no approved vaccines or antiviral therapies for the prevention or treatment of alphavirus infection.

Mayaro virus (MAYV) is an arthritogenic alphavirus that was first isolated in 1954 in Trinidad, and recent outbreaks have been reported in numerous areas of Central and South America (11, 12). The primary vectors for MAYV are Haemagogus spp. mosquitoes, which transmit the virus to primates in a sylvatic cycle. However, MAYV vector competence studies have demonstrated transmission potential in multiple Aedes and Anopheles mosquitoes (13-17). The wide range and distribution of MAYV-competent vectors underscores the risk of potential urban transmission (18) and global spread (19).

The alphavirus glycoprotein is composed of heterodimers of two transmembrane subunits, E2 and E1, which mediate viral attachment and membrane fusion, respectively (20-22). The pre-fusion E2/E1 heterodimer forms a trimeric spike that is arranged in an icosahedral lattice on the viral particle. E2 is initially expressed as a precursor polypeptide known as p62. During virus biogenesis, p62 is processed by cellular furin to generate E2 and the peripheral E3 polypeptide. E3 remains bound to the E2/E1 heterodimer during exocytic transport and prevents premature conformational changes and membrane fusion (23, 24). The release of E3 is the final step of virus maturation and primes the glycoprotein for membrane fusion.

Both E2 and E1 proteins are targets of the neutralizing antibody response. Antibody-mediated protection by neutralizing mAbs has been shown against several alphaviruses (25-31). The isolation of potent and protective neutralizing CHIKV mAbs targeting regions of E2, such as the β-connector region and the A domain has been reported (25-27). These mAbs neutralize virus via multiple mechanisms, including prevention of attachment and membrane fusion. The alphavirus receptor Mxra8 binds to regions spanning the A and B domains of E2 protein (32), and neutralizing mAbs targeting these regions can effectively disrupt virus interaction with the host receptor (33).

Many of the identified neutralizing human mAbs against alphaviruses are virus-specific and do not inhibit heterologous alphaviruses. Notably, most of these mAbs target CHIKV, and there are few examples of MAYV-reactive human mAbs. Recent work has demonstrated cross-reactivity and cross-neutralization of human polyclonal sera to heterologous alphaviruses (34-36), suggesting that broadly-reactive and/or broadly-neutralizing monoclonal antibodies (bNAbs) may be elicited by alphavirus infection in humans. While a number of murine bNAbs have been characterized (30, 37, 38), few human bNAbs that engage multiple alphaviruses have been described (33). For example, the murine mAb CHK-265 can protect against CHIKV, MAYV and RRV challenge in mice (38). More recently, a human mAb RRV-12 was shown to protect mice against RRV and MAYV infection (33). Both CHK-265 and RRV-12 broadly neutralize infection by engaging the B domain of E2, but whether such protective alphavirus bNAbs are elicited commonly during the course of human CHIKV infection is unknown.

### SUMMARY OF THE INVENTION

The present disclosure describes the isolation and characterization of cross-reactive alphavirus mAbs from a CHIKV convalescent donor. A single B cell sorting strategy using a heterologous MAYV antigen was employed to isolate 33 cross-reactive mAbs and found that they target multiple epitopes on the E1 and E2 proteins. Five human bNAbs that neutralize CHIKV, MAYV and other alphaviruses with differing potencies were identified. Epitope binning and viral escape studies suggest that human bNAbs target related but distinct regions of the B domain of E2. Remarkably, sequence analysis of human bNAbs showed few somatic mutations, and inferred germline variants largely retained neutralizing function. Two bNAbs demonstrated protection against both CHIKV- and MAYV-induced musculoskeletal disease in mice. Together, these studies further define heterologous humoral immunity among related alphaviruses in humans as well as the determinants of antibody-mediated cross-protection.

An anti-alphavirus antibody or alphavirus-binding fragment thereof, wherein said antibody or fragment thereof comprises:
(1) a heavy chain comprising the CDRS set forth in GFTFSNYA (SEQ ID NO:1), ISFDGSIN (SEQ ID NO:2), CARDRYYYDSSAYFLIDAFDI (SEQ ID NO:3), and a light chain comprising the CDRS set forth in QSVSSH (SEQ ID NO:4), DAS (SEQ ID NO: 5), and QQRSNWPPIT (SEQ ID NO:6);
(2) a heavy chain comprising the CDRS set forth in GFILSDHY (SEQ ID NO:7), SRNKAKRYTT (SEQ ID NO:8), and TRAGYYDKNGDSLDALDI (SEQ ID NO:9), and a light chain comprising the CDRS set forth in QTVSSN (SEQ ID NO:10), GIS (SEQ ID NO: 11), and QQSYSLPRT (SEQ ID NO:12);
(3) a heavy chain comprising the CDRS set forth in GYSFTSHT (SEQ ID NO:13), INTNTGNP (SEQ ID NO:14), and ARVLKCLGGSASCSGHGYYDYGMAV (SEQ ID NO: 15), and a light chain comprising the CDRS set forth in QSLLHSNGYNF (SEQ ID NO: 16), LGS (SEQ ID NO:17), and MQALQTFMYT (SEQ ID NO:18);
(4) a heavy chain comprising the CDRS set forth in GFTFRDYW (SEQ ID NO:19), INRNGNEK (SEQ ID NO:20), and VRDSSPSFGPGNYYDAFDI (SEQ ID NO:21), and a light chain comprising the CDRS set forth in QDIRNE (SEQ ID NO:22), AAS (SEQ ID NO: 23), and LQDFNYPRT (SEQ ID NO:24);
(5) a heavy chain comprising the CDRS set forth in GYTFSGYY (SEQ ID NO:25), INPNSGGT (SEQ ID NO:26), and SRDPGYTYGYPLGY (SEQ ID NO:27), and a light chain comprising the CDRS set forth in QSVSSSY (SEQ ID NO:28), GTS (SEQ ID NO: 29), and QQYGNSPPYT (SEQ ID NO:30);
(6) a heavy chain comprising the CDRS set forth in GFTIIDSY (SEQ ID NO:31), INPSGSVI (SEQ ID NO:32), and ARGIYDQSDAFDL (SEQ ID NO:33), and a light chain comprising the CDRS set forth in QGISNS (SEQ ID NO:34), GAS (SEQ ID NO: 35), and QQYYITPPMT (SEQ ID NO:36); or
(7) a heavy chain comprising the CDRS set forth in GSTFISHA (SEQ ID NO:37), LIPVSGTP (SEQ ID NO:38), and ATWDADSTTLLYYFMDV (SEQ ID NO:39), and a light chain comprising the CDRS set forth in QSISRW (SEQ ID NO:40), KAS (SEQ ID NO: 41), and QQYNSYSTWT (SEQ ID NO:42).

An anti-alphavirus antibody or alphavirus-binding fragment thereof, wherein said antibody or fragment thereof comprises:
(1) a heavy chain variable domain set forth in SEQ ID NO:199, and a light chain variable domain set forth in SEQ ID NO:200;
(2) a heavy chain variable domain set forth in SEQ ID NO:201, and a light chain variable domain set forth in SEQ ID NO:202;
(3) a heavy chain variable domain set forth in SEQ ID NO:203, and a light chain variable domain set forth in SEQ ID NO:204;
(4) a heavy chain variable domain set forth in SEQ ID NO:205, and a light chain variable domain set forth in SEQ ID NO:206;
(5) a heavy chain variable domain set forth in SEQ ID NO:207, and a light chain variable domain set forth in SEQ ID NO:208;
(6) a heavy chain variable domain set forth in SEQ ID NO:209, and a light chain variable domain set forth in SEQ ID NO:210;
(7) a heavy chain variable domain set forth in SEQ ID NO:211, and a light chain variable domain set forth in SEQ ID NO:212.

An anti-alphavirus antibody or alphavirus-binding fragment thereof, wherein said antibody or fragment thereof comprises:
(1) a heavy chain comprising the CDRS set forth in GFTFSNYA (SEQ ID NO:1), ISFDGSIN (SEQ ID NO:2), CARDRYYYDSSAYFLIDAFDI (SEQ ID NO:3); and
(2) a light chain comprising the CDRS set forth in QSVSSH (SEQ ID NO:4), DAS (SEQ ID NO: 5), and QQRSNWPPIT (SEQ ID NO:6).

An anti-alphavirus antibody or alphavirus-binding fragment thereof, wherein said antibody or fragment thereof comprises:
(1) a heavy chain variable domain set forth in SEQ ID NO:199; and
(2) a light chain variable domain set forth in SEQ ID NO:200.

A method for treating an alphavirus infection in a subject, comprising administering an antibody or antigen-binding fragment thereof as described herein in an amount effective to treat the alphavirus infection in the subject. In some embodiments, the alphavirus is selected from the group consisting of Chikungunya virus (CHIKV), Mayaro virus (MAYV), Ross River virus (RRV), O'nyong-nyong virus (ONNV), and Semliki Forest virus (SFV).

A method for inhibiting an alphavirus infection in a subject, comprising administering an antibody or antigen-binding fragment thereof as described herein in an amount effective to inhibit the alphavirus infection in the subject. In some embodiments, the alphavirus is selected from the group consisting of Chikungunya virus (CHIKV), Mayaro virus (MAYV), Ross River virus (RRV), O'nyong-nyong virus (ONNV), and Semliki Forest virus (SFV).

An isolated nucleic acid molecule encoding the antibody, or binding fragment thereof, as described herein.

A vector comprising the nucleic acid molecule as described herein.

A host cell comprising the nucleic acid molecule as described herein, or the vector as described herein.

A method of producing an anti-alphavirus antibody comprising culturing the host cell of as described herein, under conditions wherein the anti-alphavirus antibody is produced by the host cell.

A pharmaceutical composition comprising an anti-alphavirus antibody, or alphavirus-binding fragment thereof, as described herein, and a pharmaceutically acceptable excipient.

A method of reducing an activity of an alphavirus in a subject in need thereof, comprising administering to said subject a therapeutically effective amount of the anti-alphavirus antibody, or alphavirus-binding fragment thereof, as described herein, or the pharmaceutical composition as described herein.

A method of treating a disease, disorder, or condition mediated by, or related to increased activity of an alphavirus in a subject a therapeutically effective amount of the anti-alphavirus antibody, or alphavirus-binding fragment thereof, as described herein, or the pharmaceutical composition as described herein.

An assay device is provided for selectively detecting an alphavirus in a biological sample comprising:
a first portion comprising a first plurality of anti-alphavirus antibodies as described herein,
wherein the antibodies are each attached to their own reporting entity;
a second portion comprising a second plurality of anti-alphavirus antibodies.

In some embodiments, the alphavirus is selected from the group consisting of Chikungunya virus (CHIKV), Mayaro virus (MAYV), Ross River virus (RRV), O'nyong-nyong virus (ONNV), and Semliki Forest virus (SFV).

### BRIEF DESCRIPTION OF THE DRAWINGS

FIGS. 1A-1F show design, expression, and purification of MAYV p62-E1 heterodimer for single B cell sorting. FIGS. 1A shows structural organization of the alphavirus glycoprotein. FIG. 1B shows schematic of MAYV structural protein and p62-E1 construct. Arrows indicate sites of proteolytic cleavage. MAYV p62-E1 contains a mutated furin cleavage site, a glycine-serine linker connecting p62 to E1, and a C-terminal strep II tag for purification. FIG. 1B discloses SEQ ID NO: 215. FIG. 1C shows immunoblot and FIG. 1D shows SDS-PAGE of MAYV p62-E1 generated in S2 cells. MAYV p62-E1 was detected by an anti-E1/E2 antibody. FIG. 1E shows size exclusion chromatogram of MAYV p62-E1. Sample was run on Superdex S200 10/300 AKTA column. A retention time of 13.5 mL was observed, consistent with MW of ˜100 kDa. FIG. 1F shows ELISA reactivity of CHK-265 toward CHIKV/MAYV p62-E1 (left) and serological ELISA (right) of patient DC2 serum toward CHIKV/MAYV p62-E1. ELISAs were performed twice independently in triplicate wells (mean±SD).

FIGS. 2A-2E show sequence and reactivity profiles of mAbs isolated from MAYV-reactive B cells. FIG. 2A shows ELISA three-point screen of 71 human mAbs for reactivity against CHIKV and MAYV glycoproteins. All mAbs were tested at 300 nM, 30 nM, and 3 nM concentrations against CHIKV p62-E1, MAYV p62-E1 and 1% BSA control. Experiment was performed twice independently in triplicate wells.—FIG. 2B shows IGHV and IGKV gene usage of 33 cross-reactive mAbs. FIG. 2C shows distribution of HCDR3 lengths of 33 cross-reactive mAbs. FIG. 2D shows summary of CHIKV E1 E2 ELISA reactivity of cross-reactive mAbs. Mabs that showed signal (Abs 450 nm) greater than 5-fold relative to BSA control at 30 nM (E1′) or 16.6 nM (E2) were considered reactive. Experiment was performed three times independently in triplicate wells. FIG. 2E shows range of EC50 values of cross-reactive mAbs for CHIKV and MAYV p62-E1. Values were determined by eight-point serial dilution series for each mAb. MAbs are colored according to reactivity to E1′ (red), E2 (blue) or both (green). Experiment was performed twice independently in duplicate wells.

FIGS. 3A-3C show identification of cross-neutralizing mAbs targeting multiple alphaviruses. FIG. 3A shows two-point neutralization screen against CHIKV (181/25) and MAYV (Guyane). Focus reduction neutralization tests (FRNT) were performed at 300 nM and 30 nM for all 71 human mAbs. Highlighted are mAbs that demonstrated >80% inhibition of both viruses at only 300 nM (black) or both 300 nM and 30 nM (colored as indicated). Values for each mAb represent the mean of the % inhibition of two independent experiments performed in triplicate wells relative to untreated control. FIG. 3B shows full neutralization curves of cross-neutralizing mAbs. FRNT was performed with serially diluted mAbs at as indicated. Viruses tested were CHIKV 181/25, MAYV TRVL-4675, ONNV SG650, RRV T48 and SFV 88/11. Shown are experiments performed twice independently in triplicate (points represent mean±SD) FIG. 3C shows IC50 values of human bNAbs. Values shown are in nM and were determined by fitting nonlinear regression to FRNTs. 95% confidence interval is shown for each curve.

FIGS. 4A-4C show binding kinetic profiles of cross-reactive mAbs and epitope binning. FIG. 4A shows binding of E1 and E2 reactive mAbs to CHIKV and MAYV glycoproteins by BLI. A representative dataset from two independent experiments is shown. FIG. 4B shows kinetic analysis of cross-reactive mAbs by BLI. A representative dataset from two independent experiments is shown. FIG. 4C shows two-phase binding of cross-reactive mAbs to MAYV p62-E1 against CHK-265 by BLI. MAb CHK-265 was bound to a sensor loaded with MAYV p62-E1 followed by the sequential addition of the indicated second mAb. Representative data from two independent experiments is shown.

FIGS. 5A-5C show generation of escape mutants of human bNAbs DC2.M108 and DC2.M357. FIG. 5A shows neutralization curves of DC2.M16, DC2.M108 and DC2.M357 bNAbs against wild-type and mutant rVSV-CHIKV. Data shown are from two independent experiments performed in triplicate wells (points represent mean±SD). IC50 values were determined by fitting a non-linear regression and 95% confidence interval is shown. FIG. 5B shows escape mutations for DC2.M108 and DC2.M357 mapped to the CHIKV p62-E1 heterodimer structure (PDB ID: 3N40) and the FIG. 5C shows E1/E2 heterohexamer (PDB ID: 3J2W). Cα spheres indicate positions of escape mutations for DC2.M108 (cyan), DC2.M357 (magenta), as well as CHK-265 escape mutations against RRV (silver). E1 is colored gray.

FIGS. 6A-6E show germline sequence analysis and functional characteristics of inferred germline bNAbs. FIG. 6A shows number of V-gene nucleotide substitutions for cross-reactive mAbs. BNAbs are highlighted as indicated. Mean and SD are shown in gray bars. FIG. 6B shows alignment of mAbs DC2.M16, DC2.M131, DC2.M108 and DC2.M230 to the IGHV3-11/IGKV1-NL1 germline sequence (SEQ ID NOS 216-225, respectively, in order of appearance). Mutations shared by DC2.M108 and DC2.M230 are highlighted in blue. FIG. 6C shows binding of inferred germline mAbs DC2.M16gL and DC2.M108gL to CHIKV and MAYV p62-E1 proteins by BLI. FIG. 6D shows neutralization of CHIKV and MAYV by inferred germline mAbs. Shown are two independent experiments performed in triplicate wells (points represent mean±SD). IC₅₀ values were determined by fitting a non-linear regression and 95% confidence interval is shown. FIG. 6E shows neutralization of ONNV by DC2.M16 after introduction of key mutations. Mutations shared by DC2.M108 and DC2.M230 were incorporated to DC2.M16 sequence. Shown are two independent experiments performed in triplicate wells (points represent mean±SD). IC50 values were determined by fitting a non-linear regression and 95% confidence interval is shown.

FIGS. 7A-7C show antibody protection of CHIKV and MAYV-induced musculoskeletal disease in vivo. FIG. 7A shows CHIKV and MAYV-induced joint swelling in mAb treated mice. 4-week-old C57BL/6J mice were administered 200 μg (10 mg/kg) of DC2.M16, DC2.M357 or isotype control mAb by intraperitoneal route one day before subcutaneous inoculation of either 103 FFU of CHIKV-LR2006-OPY1 or MAYV-BeH407 in the footpad. Swelling was measured in the ipsilateral ankle using digital calipers over 14 days. Two independent experiments were performed, with n=10 mice per antibody for each group (two-way ANOVA with Tukey's post-test) (FIG. 7B) CHIKV-LR2006-OPY1 and (FIG. 7C) MAYV-BeH407 viral titers in ankle, calf muscle, spleen and draining lymph node were determined 3 days post-inoculation. Viral RNA levels were measured by qRT-PCR by qRT-PCR and compared to a viral RNA standard curve generated from titered viral stock to determine FFU equivalents. Two independent experiments were performed, with n=6 mice per antibody for each group (one-way ANOVA with Dunnett's post test). *, P<0.05; **, P<0.01; ***, P<0.001, ****, P<0.0001 compared to isotype control.

FIG. 8 shows MAYV-induced (ipsilateral and contralateral) ankle swelling, or CHIKV-induced ipsilateral ankle swelling in mice pretreated with DC2.M16, DC2.M357 or isotype control.

FIG. 9 shows viremia in tissues of mice challenged with CHIKV and pretreated with DC2.M16, DC2.M357 or isotype control.

FIG. 10 shows viremia in tissues of mice challenged with MAYV and pretreated with DC2.M16, DC2.M357 or isotype control.

FIG. 11 shows gating strategy for isolation of MAYV-reactive memory B cells from a CHIKV convalescent donor. Representative flow cytometric gating of PBMCs is shown. Cells were filtered for size and granularity. CD3+, CD8+ and CD14+ cells were excluded and CD20+ CD27+ IgG+ MAYV p62-E1+ B cells were collected in individual wells.

FIG. 12 shows FRNT of human mAbs with SINV. FRNT was performed with serially diluted mAbs at nine different concentrations as indicated. Shown are two independent experiments performed in triplicate wells (points represent mean±SD). IC50 values were determined by fitting a non-linear regression and 95% confidence interval is shown.

FIGS. 13A-13B show monovalent binding kinetics of cross-reactive Fabs by BLI. FIG. 13A shows binding of Fabs DC2.M16, DC2.M108 and DC2.M357 to CHIKV and MAYV p62-E1 glycoproteins by BLI. FIG. 13B shows kinetic analysis of cross-reactive Fabs. A representative data set from two independent experiments is shown.

FIG. 14 shows two-phase CHK-265 binning by BLI. Two-phase binding of cross-reactive mAbs to MAYV p62-E1 vs. CHK-265 by BLI. Cross-reactive mAb was first bound to a sensor loaded with MAYV p62-E1 and then CHK-265 was added as indicated. Representative data from two independent experiments are shown.

FIGS. 15A-15B show sequence relation and alignment of arthritogenic alphaviruses. FIG. 15A shows phylogenetic tree based on the structural polyprotein amino acid sequences of indicated alphaviruses was generated using ClustalW2. The percentage identity to CHIKV AF15561 is indicated. FIG. 15B shows alignment of B domain amino acid sequence of arthritogenic alphaviruses (SEQ ID NOS 226-234, respectively, in order of appearance). Alignment was generated using ESPript 3.0. Asterisks indicate escape mutations identified for DC2.M108 (cyan) and DC2.357 (magenta).

FIGS. 16A-16C show DC2.M357 inferred germline sequence alignment, binding and neutralization. FIG. 16A shows alignment of DC2.M357 to IGHV3-30/IGKV3-11 germline sequence (SEQ ID NOS 235-238, respectively, in order of appearance). FIG. 16B shows binding of inferred germline mAbs DC2.M16gL, DC2.M108gL and DC2.M357gL to CHIKV and MAYV p62-E1 by ELISA. Data are from two independent experiments performed in triplicate wells (points represent mean±SD). FIG. 16C shows neutralization of CHIKV 181/25 and MAYV TRVL 4675 by DC2.M357gL. Data are from two independent experiments performed in triplicate wells (points represent mean±SD).

### DETAILED DESCRIPTION OF THE INVENTION

Arthritogenic alphaviruses are globally distributed, mosquito-transmitted viruses that cause rheumatological disease in humans and include Chikungunya virus (CHIKV), Mayaro virus (MAYV), and others. Although serological evidence suggests that some antibody-mediated heterologous immunity may be afforded by alphavirus infection, the extent to which broadly neutralizing antibodies that protect against multiple arthritogenic alphaviruses are elicited during natural infection remains unknown. Here, the isolation and characterization of MAYV-reactive alphavirus mAbs from a CHIKV convalescent donor were described. 33 human monoclonal antibodies (mAbs) that cross-reacted with CHIKV and MAYV and engaged multiple epitopes on the E1 and E2 glycoproteins were characterized. five mAbs that target distinct regions of the B domain of E2 and potently neutralize multiple alphaviruses with differential breadth of inhibition were identified. These broadly neutralizing mAbs contain few somatic mutations, and inferred germline-revertants retained neutralizing capacity. Two bNAbs, DC2.M16 and DC2.M357, protected against both CHIKV- and MAYV-induced musculoskeletal disease in mice. These findings enhance understanding of the cross-reactive and cross-protective antibody response to human alphavirus infections.

Arthritogenic alphaviruses such as Chikungunya and Mayaro viruses cause febrile illness, rash, and a debilitating chronic polyarthritis in humans. Currently, there are no approved vaccines or antiviral therapies for the prevention or treatment of alphavirus infection. Here, 33 mAbs from a CHIKV convalescent donor that cross-react with other arthritogenic alphaviruses were identified and characterized. It was demonstrated that five broadly neutralizing mAbs can inhibit multiple arthritogenic alphaviruses and map their epitopes through binding and viral escape mutant analysis. Finally, it was shown that two mAbs, DC2.M16 and DC2.M357, protect against alphavirus disease in mice. These studies inform how the human immune system combats alphavirus infection and can guide the development of new antiviral treatments and vaccines.

An anti-alphavirus antibody or alphavirus-binding fragment thereof, wherein said antibody or fragment thereof comprises:
(1) a heavy chain comprising the CDRS set forth in GFTFSNYA (SEQ ID NO:1), ISFDGSIN (SEQ ID NO:2), CARDRYYYDSSAYFLIDAFDI (SEQ ID NO:3), and a light chain comprising the CDRS set forth in QSVSSH (SEQ ID NO:4), DAS (SEQ ID NO: 5), and QQRSNWPPIT (SEQ ID NO:6);
(2) a heavy chain comprising the CDRS set forth in GFILSDHY (SEQ ID NO:7), SRNKAKRYTT (SEQ ID NO:8), and TRAGYYDKNGDSLDALDI (SEQ ID NO:9), and a light chain comprising the CDRS set forth in QTVSSN (SEQ ID NO:10), GIS (SEQ ID NO: 11), and QQSYSLPRT (SEQ ID NO:12);
(3) a heavy chain comprising the CDRS set forth in GYSFTSHT (SEQ ID NO:13), INTNTGNP (SEQ ID NO:14), and ARVLKCLGGSASCSGHGYYDYGMAV (SEQ ID NO: 15), and a light chain comprising the CDRS set forth in QSLLHSNGYNF (SEQ ID NO: 16), LGS (SEQ ID NO:17), and MQALQTFMYT (SEQ ID NO:18);
(4) a heavy chain comprising the CDRS set forth in GFTFRDYW (SEQ ID NO:19), INRNGNEK (SEQ ID NO:20), and VRDSSPSFGPGNYYDAFDI (SEQ ID NO:21), and a light chain comprising the CDRS set forth in QDIRNE (SEQ ID NO:22), AAS (SEQ ID NO: 23), and LQDFNYPRT (SEQ ID NO:24);
(5) a heavy chain comprising the CDRS set forth in GYTFSGYY (SEQ ID NO:25), INPNSGGT (SEQ ID NO:26), and SRDPGYTYGYPLGY (SEQ ID NO:27), and a light chain comprising the CDRS set forth in QSVSSSY (SEQ ID NO:28), GTS (SEQ ID NO: 29), and QQYGNSPPYT (SEQ ID NO:30);
(6) a heavy chain comprising the CDRS set forth in GFTIIDSY (SEQ ID NO:31), INPSGSVI (SEQ ID NO:32), and ARGIYDQSDAFDL (SEQ ID NO:33), and a light chain comprising the CDRS set forth in QGISNS (SEQ ID NO:34), GAS (SEQ ID NO: 35), and QQYYITPPMT (SEQ ID NO:36); or
(7) a heavy chain comprising the CDRS set forth in GSTFISHA (SEQ ID NO:37), LIPVSGTP (SEQ ID NO:38), and ATWDADSTTLLYYFMDV (SEQ ID NO:39), and a light chain comprising the CDRS set forth in QSISRW (SEQ ID NO:40), KAS (SEQ ID NO: 41), and QQYNSYSTWT (SEQ ID NO:42).

An anti-alphavirus antibody or alphavirus-binding fragment thereof, wherein said antibody or fragment thereof comprises:
(1) a heavy chain variable domain set forth in SEQ ID NO:199, and a light chain variable domain set forth in SEQ ID NO:200;
(2) a heavy chain variable domain set forth in SEQ ID NO:201, and a light chain variable domain set forth in SEQ ID NO:202;
(3) a heavy chain variable domain set forth in SEQ ID NO:203, and a light chain variable domain set forth in SEQ ID NO:204;
(4) a heavy chain variable domain set forth in SEQ ID NO:205, and a light chain variable domain set forth in SEQ ID NO:206;
(5) a heavy chain variable domain set forth in SEQ ID NO:207, and a light chain variable domain set forth in SEQ ID NO:208;
(6) a heavy chain variable domain set forth in SEQ ID NO:209, and a light chain variable domain set forth in SEQ ID NO:210;
(7) a heavy chain variable domain set forth in SEQ ID NO:211, and a light chain variable domain set forth in SEQ ID NO:212.

An anti-alphavirus antibody or alphavirus-binding fragment thereof, wherein said antibody or fragment thereof comprises:
(1) a heavy chain comprising the CDRS set forth in GFTFSNYA (SEQ ID NO:1), ISFDGSIN (SEQ ID NO:2), CARDRYYYDSSAYFLIDAFDI (SEQ ID NO:3); and
(2) a light chain comprising the CDRS set forth in QSVSSH (SEQ ID NO:4), DAS (SEQ ID NO: 5), and QQRSNWPPIT (SEQ ID NO:6).

An anti-alphavirus antibody or alphavirus-binding fragment thereof, wherein said antibody or fragment thereof comprises:
(1) a heavy chain variable domain set forth in SEQ ID NO:199; and
(2) a light chain variable domain set forth in SEQ ID NO:200.

In some embodiments, the antibody comprises a non-naturally occurring Fc region. In some embodiments, the antibody comprises a mutated human Fc region. In some embodiments, the antibody is an Immunoglobulin G type antibody.

In some embodiments, the antibody comprises antibody, or alphavirus-binding fragment thereof, binds an alphavirus with a binding affinity (K_{D}) of from about 0.005 nM to 100 nM, from 0.25 nM to 25 nM, from 0.5 nM to 15 nM, from 0.7 nM to 16 nM, from 1 nM to 10 nM, or from 0.5 nM to 4.4 nM.

In some embodiments, the antibody comprises antibody, or alphavirus-binding fragment thereof, is a monoclonal antibody.

In some embodiments, the antibody comprises antibody, or alphavirus-binding fragment thereof, is a recombinant antibody.

In some embodiments, the alphavirus-binding fragment comprises an Fab, F(ab)2 or scFv.

In some embodiments, the antibody, or alphavirus-binding fragment thereof binds to E2.

In some embodiments, the antibody, or alphavirus-binding fragment thereof binds to B domain of E2.

In some embodiments, the alphavirus is selected from the group consisting of Chikungunya virus (CHIKV), Mayaro virus (MAYV), Ross River virus (RRV), O'nyong-nyong virus (ONNV), and Semliki Forest virus (SFV).

A method for treating an alphavirus infection in a subject, comprising administering an antibody or antigen-binding fragment thereof as described herein in an amount effective to treat the alphavirus infection in the subject. In some embodiments, the alphavirus is selected from the group consisting of Chikungunya virus (CHIKV), Mayaro virus (MAYV), Ross River virus (RRV), O'nyong-nyong virus (ONNV), and Semliki Forest virus (SFV).

A method for inhibiting an alphavirus infection in a subject, comprising administering an antibody or antigen-binding fragment thereof as described herein in an amount effective to inhibit the alphavirus infection in the subject. In some embodiments, the alphavirus is selected from the group consisting of Chikungunya virus (CHIKV), Mayaro virus (MAYV), Ross River virus (RRV), O'nyong-nyong virus (ONNV), and Semliki Forest virus (SFV).

In some embodiments, the antibody, or alphavirus-binding fragment thereof binds to E2.

In some embodiments, the antibody, or alphavirus-binding fragment thereof binds to B domain of E2.

In some embodiments, the antibody, or antigen-binding fragment thereof binds Chikungunya virus E2, Chikungunya virus p62-E1 hybrid protein, Chikungunya virus E1-E2 glycoprotein, Mayaro virus E2, Mayaro virus p62-E1 hybrid protein or Mayaro virus E1-E2 glycoprotein.

In some embodiments, the method is for treating or inhibiting Chikungunya virus infection.

In some embodiments, the method is for treating or inhibiting Mayaro virus infection.

In some embodiments, the method is for treating or inhibiting O'nyong'nyong virus infection.

In some embodiments, the method is for treating or inhibiting Ross River virus infection.

In some embodiments, the method is for treating or inhibiting Semliki Forest virus infection.

An isolated nucleic acid molecule encoding the antibody, or antigen-binding fragment thereof, as described herein. In some embodiments, the isolated nucleic acid molecule is DNA. In some embodiments, the isolated nucleic acid molecule is cDNA.

A vector comprising the nucleic acid molecule as described herein.

A host cell comprising the nucleic acid molecule as described herein, or the vector as described herein.

A method of producing an anti-alphavirus antibody comprising culturing the host cell of as described herein, under conditions wherein the anti-alphavirus antibody is produced by the host cell.

A pharmaceutical composition comprising an anti-alphavirus antibody, or alphavirus-binding fragment thereof, as described herein, and a pharmaceutically acceptable excipient.

A method of reducing an activity of alphavirus in a subject in need thereof, comprising administering to said subject a therapeutically effective amount of the anti-alphavirus antibody, or alphavirus-binding fragment thereof, as described herein, or the pharmaceutical composition as described herein.

A method of treating a disease, disorder, or condition mediated by, or related to increased activity of an alphavirus in a subject a therapeutically effective amount of the anti-alphavirus antibody, or alphavirus-binding fragment thereof, as described herein, or the pharmaceutical composition as described herein.

An assay device is provided for selectively detecting an alphavirus in a biological sample comprising:
a first portion comprising a first plurality of anti-alphavirus antibodies as described herein,
wherein the antibodies are each attached to their own reporting entity;
a second portion comprising a second plurality of anti-alphavirus antibodies.

In some embodiments, the antibody is a monoclonal antibody, or the fragment thereof is a fragment of a monoclonal antibody.

In some embodiments, the reporting entity comprises a gold nanoparticle. In some embodiments, the reporting entity comprises an enzyme. In some embodiments, the second plurality of anti-alphavirus antibodies is affixed to a solid support of the device. In some embodiments, the first plurality of anti-alphavirus antibodies is not affixed to a solid support of the device. In some embodiments, the solid support comprises nitrocellulose. In some embodiments, the assay device further comprises a fluid sample pad prior in sequential order to the first and second portions. In some embodiments, the assay device further comprises a control portion subsequent in sequential order to the first and second portions. In some embodiments, the control portion comprises a third plurality of antibodies, immobilized on a solid support of the device, and which third plurality of antibodies are capable of binding the first plurality of anti-alphavirus antibodies each attached to their own reporting molecule. In some embodiments, the assay device further comprises a fluid-absorbent wicking pad subsequent in sequential order to the first and second portions, and third portion if present.

A vaccine composition is provided comprising an anti-alphavirus antibody, or alphavirus-binding fragment thereof, and a carrier. In some embodiments, the vaccine further comprises an immunological adjuvant.

A method is provided of detecting an alphavirus in a biological sample comprising contacting the device described herein with the sample and observing if alphavirus-bound antibodies bind to the second plurality of alphavirus-binding antibodies, wherein if such antibodies bind then alphavirus has been detected in the biological sample and wherein if no alphavirus-bound antibodies bind to the second plurality of alphavirus-binding antibodies then alphavirus has not been detected in the biological sample.

In some embodiments, the method further comprises obtaining the sample from a subject.

In some embodiments, the sample is urine or blood. In some embodiments, the subject is human.

As used herein, the term “antibody” refers to an intact antibody, i.e. with complete Fc and Fv regions. “Fragment” refers to any portion of an antibody, or portions of an antibody linked together, such as, in non-limiting examples, a Fab, F(ab)2, a single-chain Fv (scFv), which is less than the whole antibody but which is an antigen-binding portion and which competes with the intact antibody of which it is a fragment for specific binding. In this case, the antigen is locate on the alphavirus.

As such a fragment can be prepared, for example, by cleaving an intact antibody or by recombinant means. See generally, Fundamental Immunology, Ch. 7 (Paul, W., ed., 2nd ed. Raven Press, N.Y. (1989), hereby incorporated by reference in its entirety). Antigen-binding fragments may be produced by recombinant DNA techniques or by enzymatic or chemical cleavage of intact antibodies or by molecular biology techniques. In some embodiments, a fragment is an Fab, Fab′, F(ab′)2, Fd, Fv, complementarity determining region (CDR) fragment, single-chain antibody (scFv), (a variable domain light chain (VL) and a variable domain heavy chain (VH) linked via a peptide linker. In an embodiment, the scFv comprises a variable domain framework sequence having a sequence identical to a human variable domain FR1, FR2, FR3 or FR4. In an embodiment, the scFv comprises a linker peptide from 5 to 30 amino acid residues long. In an embodiment, the scFv comprises a linker peptide comprising one or more of glycine, serine and threonine residues.

In an embodiment the linker of the scFv is 10-25 amino acids in length. In an embodiment the peptide linker comprises glycine, serine and/or threonine residues. For example, see Bird et al., Science, 242:423-426 (1988) and Huston et al., Proc. Natl. Acad. Sci. USA, 85:5879-5883 (1988) each of which are hereby incorporated by reference in their entirety), or a polypeptide that contains at least a portion of an antibody that is sufficient to confer Mtb capsular AM-specific antigen binding on the polypeptide, including a diabody. From N-terminus to C-terminus, both the mature light and heavy chain variable domains comprise the regions FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md. (1987 and 1991)), Chothia & Lesk, J. Mol. Biol. 196:901-917 (1987), or Chothia et al., Nature 342:878-883 (1989), each of which are hereby incorporated by reference in their entirety). As used herein, the term “polypeptide” encompasses native or artificial proteins, protein fragments and polypeptide analogs of a protein sequence. A polypeptide may be monomeric or polymeric. As used herein, an Fd fragment means an antibody fragment that consists of the VH and CH1 domains; an Fv fragment consists of the V1 and VH domains of a single arm of an antibody; and a dAb fragment (Ward et al., Nature 341:544-546 (1989) hereby incorporated by reference in its entirety) consists of a VH domain. In some embodiments, fragments are at least 5, 6, 8 or 10 amino acids long. In other embodiments, the fragments are at least 14, at least 20, at least 50, or at least 70, 80, 90, 100, 150 or 200 amino acids long.

The term “monoclonal antibody” as used herein refers to an antibody member of a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target on an alphavirus, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins. Thus an identified monoclonal antibody can be produced by non-hybridoma techniques, e.g. by appropriate recombinant means once the sequence thereof is identified.

In an embodiment of the inventions described herein, the antibody is isolated. As used herein, the term “isolated antibody” refers to an antibody that by virtue of its origin or source of derivation has one, two, three or four of the following: (1) is not associated with naturally associated components that accompany it in its native state, (2) is free of other proteins from the same species, (3) is expressed by a cell from a different species, and (4) does not occur in nature.

As used herein, a “human antibody” unless otherwise indicated is one whose sequences correspond to (i.e. are identical in sequence to) an antibody that could be produced by a human and/or has been made using any of the techniques for making human antibodies as disclosed herein, but not one which has been made in a human. This definition of a human antibody specifically excludes a humanized antibody. A “human antibody” as used herein can be produced using various techniques known in the art, including phage-display libraries (e.g. Hoogenboom and Winter, J. Mol. Biol., 227:381 (1991); Marks et al., J. Mol. Biol., 222:581 (1991), hereby incorporated by reference in its entirety), by methods described in Cole et al., Monoclonal Antibodies and Cancer Therapy, Alan R. Liss, p. 77 (1985) (hereby incorporated by reference in its entirety); Boerner et al., J. Immunol., 147(1):86-95 (1991) (hereby incorporated by reference in its entirety), van Dijk and van de Winkel, Curr. Opin. Pharmacol., 5:368-74 (2001) (hereby incorporated by reference in its entirety), and by administering the antigen (e.g. an alphavirus protein or glycoprotein or an entity comprising such) to a transgenic animal that has been modified to produce such antibodies in response to antigenic challenge, but whose endogenous loci have been disabled, e.g., immunized xenomice (see, e.g., U.S. Pat. Nos. 5,939,598; 6,075,181; 6,114,598; 6,150,584 and 6,162,963 to Kucherlapati et al. regarding XENOMOUSE™ technology, each of which patents are hereby incorporated by reference in their entirety), e.g. VelocImmune® (Regeneron, Tarrytown, NY), e.g. UltiMab® platform (Medarex, now Bristol Myers Squibb, Princeton, NJ). See also, for example, Li et al., Proc. Natl. Acad. Sci. USA, 103:3557-3562 (2006) regarding human antibodies generated via a human B-cell hybridoma technology. See also KM Mouse® system, described in PCT Publication WO 02/43478 by Ishida et al., in which the mouse carries a human heavy chain transchromosome and a human light chain transgene, and the TC mouse system, described in Tomizuka et al. (2000) Proc. Natl. Acad. Sci. USA 97:722-727, in which the mouse carries both a human heavy chain transchromosome and a human light chain transchromosome, both of which are hereby incorporated by reference in their entirety. In each of these systems, the transgenes and/or transchromosomes carried by the mice comprise human immunoglobulin variable and constant region sequences.

In an embodiment, the antibody described herein is a recombinant human antibody. The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Other forms of humanized antibodies have one or more CDRs (CDR L1, CDR L2, CDR L3, CDR H1, CDR H2, or CDR H3) which are altered with respect to the original antibody, which are also termed one or more CDRs “derived from” one or more CDRs from the original antibody.

In an embodiment, the anti-alphavirus antibody described herein is capable of specifically binding or specifically binds an alphavirus. In an embodiment, the anti-alphavirus antibody described herein is capable of specifically binding alphavirus E1. In an embodiment, the anti-alphavirus antibody described herein is capable of specifically binding Chikungunya virus E1. As used herein, the terms “is capable of specifically binding” or “specifically binds” refers to the property of an antibody or fragment of binding to the (specified) antigen with a dissociation constant that is <1 μM, preferably <1 nM and most preferably <10 pM. In an embodiment, the Kd of the antibody (or fragment) for the antigen is better than 1.0 nM. In an embodiment, the Kd of the antibody (or fragment) for the antigen is better than 1.5 nM. An epitope that “specifically binds” to an antibody or a polypeptide is a term well understood in the art, and methods to determine such specific or preferential binding are also well known in the art. A molecular entity is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances.

The term “compete”, as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

Depending on the amino acid sequences of the constant domains of their heavy chains, antibodies (immunoglobulins) can be assigned to different classes. The antibody or fragment can be, e.g., any of an IgG, IgD, IgE, IgA or IgM antibody or fragment thereof, respectively. In an embodiment the antibody is an immunoglobulin G. In an embodiment the antibody fragment is a fragment of an immunoglobulin G. In an embodiment the antibody is an IgG1, IgG2, IgG2a, IgG2b, IgG3 or IgG4. In an embodiment the antibody comprises sequences from a human IgG1, human IgG2, human IgG2a, human IgG2b, human IgG3 or human IgG4. A combination of any of these antibodies subtypes can also be used. One consideration in selecting the type of antibody to be used is the desired serum half-life of the antibody. For example, an IgG generally has a serum half-life of 23 days, IgA 6 days, IgM 5 days, IgD 3 days, and IgE 2 days. (Abbas A K, Lichtman A H, Pober J S. Cellular and Molecular Immunology, 4th edition, W.B. Saunders Co., Philadelphia, 2000, hereby incorporated by reference in its entirety).

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites. The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any vertebrate species can be assigned to one of two clearly distinct types, called kappa (κ) and lambda (λ), based on the amino acid sequences of their constant domains.

“Framework” or “FR” residues are those variable domain residues other than the HVR residues as herein defined.

The term “hypervariable region” or “HVR” when used herein refers to the regions of an antibody variable domain which are hypervariable in sequence and/or form structurally defined loops. Generally, antibodies comprise six HVRs; three in the VH (H1, H2, H3) and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996). A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991) hereby incorporated by reference in its entirety). Chothia refers instead to the location of the structural loops (Chothia and Lesk, J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine of the Fc region may be removed, for example, during production or purification of the antibody, or by recombinantly engineering the nucleic acid encoding a heavy chain of the antibody. Accordingly, an intact antibody as used herein may be an antibody with or without the otherwise C-terminal lysine.

Compositions or pharmaceutical compositions comprising the antibodies, ScFvs or fragments of antibodies disclosed herein are preferably comprise stabilizers to prevent loss of activity or structural integrity of the protein due to the effects of denaturation, oxidation or aggregation over a period of time during storage and transportation prior to use. The compositions or pharmaceutical compositions can comprise one or more of any combination of salts, surfactants, pH and tonicity agents such as sugars can contribute to overcoming aggregation problems. Where a composition or pharmaceutical composition of the present invention is used as an injection, it is desirable to have a pH value in an approximately neutral pH range, it is also advantageous to minimize surfactant levels to avoid bubbles in the formulation which are detrimental for injection into subjects. In an embodiment, the composition or pharmaceutical composition is in liquid form and stably supports high concentrations of bioactive antibody in solution and is suitable for inhalational or parenteral administration. In an embodiment, the composition or pharmaceutical composition is suitable for intravenous, intramuscular, intraperitoneal, intradermal and/or subcutaneous injection. In an embodiment, the composition or pharmaceutical composition is in liquid form and has minimized risk of bubble formation and anaphylactoid side effects. In an embodiment, the composition or pharmaceutical composition is isotonic. In an embodiment, the composition or pharmaceutical composition has a pH or 6.8 to 7.4.

In an embodiment the ScFvs or fragments of antibodies disclosed herein are lyophilized and/or freeze dried and are reconstituted for use.

Examples of pharmaceutically acceptable carriers include, but are not limited to, phosphate buffered saline solution, sterile water (including water for injection USP), emulsions such as oil/water emulsion, and various types of wetting agents. Preferred diluents for aerosol or parenteral administration are phosphate buffered saline or normal (0.9%) saline, for example 0.9% sodium chloride solution, USP. Compositions comprising such carriers are formulated by well-known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000, the content of each of which is hereby incorporated in its entirety). In non-limiting examples, the can comprise one or more of dibasic sodium phosphate, potassium chloride, monobasic potassium phosphate, polysorbate 80 (e.g. 2-[2-[3,5-bis(2-hydroxyethoxy)oxolan-2-yl]-2-(2-hydroxyethoxy)ethoxy]ethyl (E)-octadec-9-enoate), disodium edetate dehydrate, sucrose, monobasic sodium phosphate monohydrate, and dibasic sodium phosphate dihydrate.

The antibodies, or fragments of antibodies, or compositions, or pharmaceutical compositions described herein can also be lyophilized or provided in any suitable forms including, but not limited to, injectable solutions or inhalable solutions, gel forms and tablet forms.

The term “Kd”, as used herein, is intended to refer to the dissociation constant of an antibody-antigen interaction. One way of determining the Kd or binding affinity of antibodies to alphavirus by measuring binding affinity of monofunctional Fab fragments of the antibody. (The affinity constant is the inverted dissociation constant). To obtain monofunctional Fab fragments, an antibody (for example, IgG) can be cleaved with papain or expressed recombinantly. The affinity of a fragment of an anti-alphavirus antibody can be determined by surface plasmon resonance (BIAcore3000™ surface plasmon resonance (SPR) system, BIAcore Inc., Piscataway N.J.). CM5 chips can be activated with N-ethyl-N′-(3-dimethylaminopropyl)-carbodiinide hydrochloride (EDC) and N-hydroxysuccinimide (NHS) according to the supplier's instructions. Alphavirus antigens can be diluted into 10 mM sodium acetate pH 4.0 and injected over the activated chip at a concentration of 0.005 mg/mL. Using variable flow time across the individual chip channels, two ranges of antigen density can be achieved: 100-200 response units (RU) for detailed kinetic studies and 500-600 RU for screening assays. Serial dilutions (0.1-10× estimated Kd) of purified Fab samples are injected for 1 min at 100 microliters/min and dissociation times of up to 2 h are allowed. The concentrations of the Fab proteins are determined by ELISA and/or SDS-PAGE electrophoresis using a Fab of known concentration (as determined by amino acid analysis) as a standard. Kinetic association rates (kon) and dissociation rates (koff) are obtained simultaneously by fitting the data to a 1:1 Langmuir binding model (Karlsson, R. Roos, H. Fagerstam, L. Petersson, B. (1994). Methods Enzymology 6. 99-110, the content of which is hereby incorporated in its entirety) using the BIA evaluation program. Equilibrium dissociation constant (Kd) values are calculated as koff/kon. This protocol is suitable for use in determining binding affinity of an antibody or fragment to any alphavirus antigen. Other protocols known in the art may also be used. For example, ELISA of alphavirus antigen with mAb can be used to determine the kD values. The Kd values reported herein used this ELISA-based protocol.

The term Fc domain or region herein is used to define a C-terminal region of an immunoglobulin heavy chain, including native sequence Fc regions and variant Fc regions. Although the boundaries of the Fc domain of an immunoglobulin heavy chain might vary, the human IgG heavy chain Fc domain is usually defined to stretch from an amino acid residue at position Cys226, or from Pro230, to the carboxyl-terminus thereof. The C-terminal lysine of the Fc domain may be removed, for example, by recombinantly engineering the nucleic acid encoding it.

In some embodiments, the antibody comprises an Fc domain. In an embodiment, the Fc domain has the same sequence or 99% or greater sequence similarity with a human IgG1 Fc domain. In an embodiment, the Fc domain has the same sequence or 99% or greater sequence similarity with a human IgG2 Fc domain. In an embodiment, the Fc domain has the same sequence or 99% or greater sequence similarity with a human IgG3 Fc domain. In an embodiment, the Fc domain has the same sequence or 99% or greater sequence similarity with a human IgG4 Fc domain. In an embodiment, the Fc domain is not mutated. In an embodiment, the Fc domain is mutated at the CH2-CH3 domain interface to increase the affinity of IgG for FcRn at acidic but not neutral pH (Dall'Acqua et al, 2006; Yeung et al, 2009). In an embodiment, the Fc domain has the same sequence as a human IgG1 Fc domain.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag. Other insertional variants of the antibody molecule include the fusion to the N- or C-terminus of the antibody of an enzyme or a polypeptide which increases the half-life of the antibody in the blood circulation.

Amino Acid Substitutions

| Original | Conservative | |
|---|---|---|
| Residue | Substitutions | Exemplary Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; |
| | | Norleucine |
| Leu (L) | Ile | Norleucine; Ile; Val; Met; |
| | | Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; |
| | | Norleucine |

Substantial modifications in the biological properties of the antibody are accomplished by selecting substitutions that differ significantly in their effect on maintaining (a) the structure of the polypeptide backbone in the area of the substitution, for example, as a β-sheet or helical conformation, (b) the charge or hydrophobicity of the molecule at the target site, or (c) the bulk of the side chain. Naturally occurring residues are divided into groups based on common side-chain properties:
(1) Non-polar: Norleucine, Met, Ala, Val, Leu, Ile;
(2) Polar without charge: Cys, Ser, Thr, Asn, Gln;
(3) Acidic (negatively charged): Asp, Glu;
(4) Basic (positively charged): Lys, Arg;
(5) Residues that influence chain orientation: Gly, Pro; and
(6) Aromatic: Trp, Tyr, Phe, His.

Non-conservative substitutions are made by exchanging a member of one of these classes for another class.

One type of substitution, for example, that may be made is to change one or more cysteines in the antibody, which may be chemically reactive, to another residue, such as, without limitation, alanine or serine. For example, there can be a substitution of a non-canonical cysteine. The substitution can be made in a CDR or framework region of a variable domain or in the constant region of an antibody. In some embodiments, the cysteine is canonical. Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as an Fv fragment.

A modification or mutation may also be made in a framework region or constant region to increase the half-life of an anti-alphavirus antibody. See, e.g., PCT Publication No. WO 00/09560. A mutation in a framework region or constant region can also be made to alter the immunogenicity of the antibody, to provide a site for covalent or non-covalent binding to another molecule, or to alter such properties as complement fixation, FcR binding and antibody-dependent cell-mediated cytotoxicity. According to the invention, a single antibody may have mutations in any one or more of the CDRs or framework regions of the variable domain or in the constant region.

In an embodiment, an antibody described herein is recombinantly produced. In an embodiment, the antibody is produced in a eukaryotic expression system. In an embodiment, the antibody produced in the eukaryotic expression system comprises glycosylation at a residue on the Fc portion corresponding to Asn297.

This invention also provides a composition comprising an antibody, or antigen-binding fragment thereof, as described herein. In an embodiment, the composition is a pharmaceutical composition. In an embodiment the composition or pharmaceutical composition comprising the antibody, or antigen-binding fragment thereof, described herein is substantially pure with regard to the antibody, or antigen-binding fragment thereof. A composition or pharmaceutical composition comprising the antibody, or antigen-binding fragment thereof, described herein is “substantially pure” with regard to the antibody or fragment when at least 60% to 75% of a sample of the composition or pharmaceutical composition exhibits a single species of the antibody, or antigen-binding fragment thereof. A substantially pure composition or pharmaceutical composition comprising the antibody, or antigen-binding fragment thereof, described herein can comprise, in the portion thereof which is the antibody, or antigen-binding fragment, 60%, 70%, 80% or 90% of the antibody, or antigen-binding fragment, of the single species, more usually about 95%, and preferably over 99%. Purity or homogeneity may be tested by a number of means well known in the art, such as polyacrylamide gel electrophoresis or HPLC.

In a preferred embodiment, the antibody is an IgG1 antibody. In an embodiment, the antibody is an IgG2 antibody. In an embodiment, the antibody is an IgG3 antibody. In an embodiment, the antibody is an IgG4 antibody.

In an embodiment, the antibody comprises the following Fc region sequence:

(SEQ ID NO: 213)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGRPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In some embodiments, the Fc region of the antibody comprises one or more Xtend mutations, for example: M428L/N434S.

In some embodiments, the Fc region of the antibody comprises one or more YTE mutations, for example: M252Y/S254T/T256E.

TABLE 1
Exemplary CDRs of the antibodies are set forth in the following table:

| Laboratory | | | |
|---|---|---|---|
| Designation | CDR1 | CDR2 | CDR3 |
| DC2_M357_HC | GFTFSNYA (SEQ | ISFDGSIN | CARDRYYYDSSAYFLIDAFD |
| | ID NO: 1) | (SEQ ID NO: 2) | I |
| | | | (SEQ ID NO: 3) |
| DC2_M357_LC | QSVSSH | DAS | QQRSNWPPIT |
| | (SEQ ID NO: 4) | (SEQ ID NO: 5) | (SEQ ID NO: 6) |
| DC2_M5_HC | GFILSDHY | SRNKAKRYTT | TRAGYYDKNGDSLDALDI |
| | (SEQ ID NO: 7) | (SEQ ID NO: 8) | (SEQ ID NO: 9) |
| DC2_M5_LC | QTVSSN | GIS | QQSYSLPRT |
| | (SEQ ID NO: 10) | (SEQ ID NO: 11) | (SEQ ID NO: 12) |
| DC2_M17_HC | GYSFTSHT | INTNTGNP | ARVLKCLGGSASCSGHGYY |
| | (SEQ ID NO: 13) | (SEQ ID NO: 14) | DYGMAV |
| | | | (SEQ ID NO: 15) |
| DC2_M17_LC | QSLLHSNGYNF | LGS | MQALQTFMYT |
| | (SEQ ID NO: 16) | (SEQ ID NO: 17) | (SEQ ID NO: 18) |
| DC2_M287_HC | GFTFRDYW | INRNGNEK | VRDSSPSFGPGNYYDAFDI |
| | (SEQ ID NO: 19) | (SEQ ID NO: 20) | (SEQ ID NO: 21) |
| DC2_M287_LC | QDIRNE | AAS | LQDFNYPRT |
| | (SEQ ID NO: 22) | (SEQ ID NO: 23) | (SEQ ID NO: 24) |
| DC2_M301_HC | GYTFSGYY | INPNSGGT | SRDPGYTYGYPLGY |
| | (SEQ ID NO: 25) | (SEQ ID NO: 26) | (SEQ ID NO: 27) |
| DC2_M301_LC | QSVSSSY | GTS | QQYGNSPPYT |
| | (SEQ ID NO: 28) | (SEQ ID NO: 29) | (SEQ ID NO: 30) |
| DC2_M336_HC | GFTIIDSY | INPSGSVI | ARGIYDQSDAFDL |
| | (SEQ ID NO: 31) | (SEQ ID NO: 32) | (SEQ ID NO: 33) |
| DC2_M336_LC | QGISNS | GAS | QQYYITPPMT |
| | (SEQ ID NO: 34) | (SEQ ID NO: 35) | (SEQ ID NO: 36) |
| DC2_M342_HC | GSTFISHA | LIPVSGTP | ATWDADSTTLLYYFMDV |
| | (SEQ ID NO: 37) | (SEQ ID NO: 38) | (SEQ ID NO: 39) |
| DC2_M342_LC | QSISRW | KAS | QQYNSYSTWT |
| | (SEQ ID NO: 40) | (SEQ ID NO: 41) | (SEQ ID NO: 42) |
| DC2_M1_HC | GFTFSSFA (SEQ ID | ISYEGKNK (SEQ ID | ARPFSMSWFEGFEF (SEQ ID |
| | NO: 43) | NO: 44) | NO: 45) |
| DC2_M1_LC | QNINSF (SEQ ID | EAS (SEQ ID NO: 47) | QQSYTAPLT (SEQ ID NO: 48) |
| | NO: 46) | | |
| DC2_M10_HC | GYTFTNYY (SEQ | IYPSGGDT (SEQ ID | ARDHLNRDSSSRGFMDY |
| | ID NO: 49) | NO: 50) | (SEQ ID NO: 51) |
| DC2_M10_LC | QSISHY (SEQ ID | DAS (SEQ ID NO: 53) | QQRGTWPPS (SEQ ID NO: 54) |
| | NO: 52) | | |
| DC2_M11_HC | GFNFNIFP (SEQ ID | ISDDVTKK (SEQ ID | ARASGWQRTGTKYYYYGM |
| | NO: 55) | NO: 56) | DV (SEQ ID NO: 57) |
| DC2_M11_LC | QDISNN (SEQ ID | DAS (SEQ ID NO: 59) | LQYDNLPYS (SEQ ID NO: 60) |
| | NO: 58) | | |
| DC2_M16_HC | GFTFSDYY | ISTSGSTM | ARGIYYQSDAFDI |
| | (SEQ ID NO: 61) | (SEQ ID NO: 62) | (SEQ ID NO: 63) |
| DC2_M16_LC | QGISNS | AAS | QQYYSTPPMT |
| | (SEQ ID NO: 64) | (SEQ ID NO: 65) | (SEQ ID NO: 66) |
| DC2_M21_HC | GYTFTSSY (SEQ | IYPSGGNT (SEQ ID | ARDHLNRDSTSRGFIDS |
| | ID NO: 67) | NO: 68) | (SEQ ID NO: 69) |
| DC2_M21_LC | QSVGNY (SEQ ID | DAS (SEQ ID NO: 71) | EQRGDWPLT (SEQ ID NO: 72) |
| | NO: 70) | | |
| DC2_M101_HC | GYTFTNYP (SEQ | INTNTGKP (SEQ ID | ARGRGATTVTTYYFDY |
| | ID NO: 73) | NO: 74) | (SEQ ID NO: 75) |
| DC2_M101_LC | QSVSSN (SEQ ID | GAS (SEQ ID NO: 77) | QHYINRPGRT (SEQ ID |
| | NO: 76) | | NO: 78) |
| DC2_M105_HC | GYTFIAFY (SEQ ID | INPYSGDT (SEQ ID | ARTVYVDKGMVMVRRLYQ |
| | NO: 79) | NO: 80) | YFGMDV (SEQ ID NO: 81) |
| DC2_M105_LC | QTVSSSY (SEQ ID | GAS (SEQ ID NO: 83) | QQYGISPEFT (SEQ ID NO: 84) |
| | NO: 82) | | |
| DC2_M108_HC | GFTFSDYF (SEQ | ISDNGNTI (SEQ ID | ARGLYIQSDAFDL (SEQ ID |
| | ID NO: 85) | NO: 86) | NO: 87) |
| DC2_M108_LC | QGLSNS (SEQ ID | AAS (SEQ ID NO: 89) | QQYYNTPPIT (SEQ ID |
| | NO: 88) | | NO: 90) |
| DC2_M109_HC | GYNFTNYW (SEQ | IYPGDSDS (SEQ ID | ARRPREQLGRLLLGDVVPH |
| | ID NO: 91) | NO: 92) | GRNDAFDI (SEQ ID NO: 93) |
| DC2_M109_LC | QSISTY (SEQ ID | SAS (SEQ ID NO: 95) | QQSYGTLWT (SEQ ID |
| | NO: 94) | | NO: 96) |
| DC2_M112_HC | GFIFKTYG (SEQ ID | IWYDGSNE (SEQ ID | ARDEAVGPYQYAAEYFHH |
| | NO: 97) | NO: 98) | (SEQ ID NO: 99) |
| DC2_M112_LC | KSVTSN (SEQ ID | GAS (SEQ ID NO: 101) | QQYNNWLT (SEQ ID |
| | NO: 100) | | NO: 102) |
| DC2_M125_HC | GFTFSSYA (SEQ | ISPSGSTI (SEQ ID | VRGVYVQSDAFDI (SEQ ID |
| | ID NO: 103) | NO: 104) | NO: 105) |
| DC2_M125_LC | QGISYS (SEQ ID | AAS (SEQ ID NO: 107) | QQYYSTPPIT (SEQ ID |
| | NO: 106) | | NO: 108) |
| DC2_M129_HC | GVTFSDYD (SEQ | IRSSGGTT (SEQ ID | VRDKDGVFDY (SEQ ID |
| | ID NO: 109) | NO: 110) | NO: 111) |
| DC2_M129_LC | QDISSW (SEQ ID | KAS (SEQ ID NO: 113) | QQYNTYPHST (SEQ ID |
| | NO: 112) | | NO: 114) |
| DC2_M131_HC | GFTFSDYY (SEQ | ISISGSTI (SEQ ID | ARGIYHQSDAFDI (SEQ ID |
| | ID NO: 115) | NO: 116) | NO: 117) |
| DC2_M131_LC | QGISNS (SEQ ID | AAS (SEQ ID NO: 119) | QQYYSTPPIT (SEQ ID |
| | NO: 118) | | NO: 120) |
| DC2_M133_HC | GFTFRDYW (SEQ | INRNGNEK (SEQ ID | VRDNSPSFGPGNYYDAFDI |
| | ID NO: 121) | NO: 122) | (SEQ ID NO: 123) |
| DC2_M133_LC | QDIRNE (SEQ ID | AAS (SEQ ID NO: 125) | LQDYNYPRT (SEQ ID |
| | NO: 124) | | NO: 126) |
| DC2_M152_HC | GYTFTDYY (SEQ | ISPKSGGT (SEQ ID | TRDNYNSWRGPDFYTGVDV |
| | ID NO: 127) | NO: 128) | (SEQ ID NO: 129) |
| DC2_M152_LC | QSVSSY (SEQ ID | NAS (SEQ ID NO: 131) | QQRSSLGLS (SEQ ID |
| | NO: 130) | | NO: 132) |
| DC2_M173_HC | GYSLTRYY (SEQ | ISPSGGGT (SEQ ID | ARDACSGGSCYTPFDY (SEQ |
| | ID NO: 133) | NO: 134) | ID NO: 135) |
| DC2_M173_LC | QSVSSN (SEQ ID | GAS (SEQ ID NO: 137) | QQYNNWPRT (SEQ ID |
| | NO: 136) | | NO: 138) |
| DC2_M192_HC | GFTFSSYG (SEQ | IWLDGTNK (SEQ ID | ARRGFHYDSSGYYYYGMDV |
| | ID NO: 139) | NO: 140) | (SEQ ID NO: 141) |
| DC2_M192_LC | QSLLHSNGYNY | LGS (SEQ ID NO: 143) | MQALQTPPFT (SEQ ID |
| | (SEQ ID NO: 142) | | NO: 144) |
| DC2_M203_HC | GFTFSNYD (SEQ | IDTSGNT (SEQ ID | VRLGGYIGNDRDAFDI (SEQ |
| | ID NO: 145) | NO: 146) | ID NO: 147) |
| DC2_M203_LC | QDISSW (SEQ ID | KAS (SEQ ID NO: 149) | QQYNTYPHST (SEQ ID |
| | NO: 148) | | NO: 150) |
| DC2_M209_HC | GFIFGDFA (SEQ ID | IRSQAHGGTT (SEQ ID | TREGVVVAARYYYYIMDV |
| | NO: 151) | NO: 152) | (SEQ ID NO: 153) |
| DC2_M209_LC | HNISRY (SEQ ID | AAS (SEQ ID NO: 155) | QQNYRTPRT (SEQ ID |
| | NO: 154) | | NO: 156) |
| DC2_M222_HC | GGIFSNYA (SEQ | FIPIVNIG (SEQ ID | ARDLEAANSVILPRLFY |
| | ID NO: 157) | NO: 158) | (SEQ ID NO: 159) |
| DC2_M222_LC | QGISNS (SEQ ID | AAS (SEQ ID NO: 161) | QQYYSTPPIT (SEQ ID |
| | NO: 160) | | NO: 162) |
| DC2_M230_HC | GFTFTDYY (SEQ | ISPSGSTI (SEQ ID NO: | ARGIYYQSDAFDT (SEQ ID |
| | ID NO: 163) | 164) | NO: 165) |
| DC2_M230_LC | QVIRNS (SEQ ID | AAS (SEQ ID NO: 167) | QQYYSTPPIT (SEQ ID NO: |
| | NO: 166) | | 168) |
| DC2_M242_HC | GYTFIDYF (SEQ ID | IYPKSGET (SEQ ID | ARDIAPTGAWWFDS (SEQ ID |
| | NO: 169) | NO: 170) | NO: 171) |
| DC2_M242_LC | QMLSSSR (SEQ ID | GAS (SEQ ID NO: 173) | QQYGSPRT (SEQ ID NO: 174) |
| | NO: 172) | | |
| DC2_M261_HC | GFTFSSHA (SEQ | ISYDGSNK (SEQ ID | VRWVAYYFDN (SEQ ID |
| | ID NO: 175) | NO: 176) | NO: 177) |
| DC2_M261_LC | QSVSSSS (SEQ ID | GTS (SEQ ID NO: 179) | QYYGSLPPIT (SEQ ID |
| | NO: 178) | | NO: 180) |
| DC2_M262_HC | GDSISSYY (SEQ ID | ISYTGST (SEQ ID | ARLGYSHPYWYFDL (SEQ |
| | NO: 181) | NO: 182) | ID NO: 183) |
| DC2_M262_LC | QSISNF (SEQ ID | AAS (SEQ ID NO: 185) | QQSYSPPLIT (SEQ ID |
| | NO: 184) | | NO: 186) |
| DC2_M266_HC | GFTFSDYY (SEQ | ISGSGKIT (SEQ ID | ARVQGEQWRGLHFDS (SEQ |
| | ID NO: 187) | NO: 188) | ID NO: 189) |
| DC2_M266_LC | QDISNY (SEQ ID | DAS (SEQ ID NO: 191) | QHRSNWPA (SEQ ID NO: 192) |
| | NO: 190) | | |
| DC2_M280_HC | GFRFGDYA (SEQ | INWDSGDI (SEQ ID | AKDSGWLRRGDYDTSGFYG |
| | ID NO: 193) | NO: 194) | PIDY (SEQ ID NO: 195) |
| DC2_M280_LC | QYISTY (SEQ ID | SAS (SEQ ID NO: 197) | QQSYGTLLT (SEQ ID |
| | NO: 196) | | NO: 198) |

TABLE 2
Exemplary variable domains of the antibodies are set forth in the following
table:

| | Variable Domain AA Sequence |
|---|---|
| DC2_M357_HC | EVQLVESGGGVVQPGRSLRLSCAASGFTFSNYALHWVRQAPGKGLEWVA |
| | VISFDGSINKYYADSVKGRFTISRDNSKNTLYLQMNSLRADGTAVYYCARD |
| | RYYYDSSAYFLIDAFDIWGQGTTVTVSS (SEQ ID NO: 199) |
| DC2_M357_LC | EIVLTQSPATLSLSPGERATLSCRASQSVSSHLAWYQQKPGQAPRLLIYDAS |
| | NRATGIPARFSGSGSGTDFTLTISSLEPEDFAIYYCQQRSNWPPITFGGGTKLE |
| | IK (SEQ ID NO: 200) |
| DC2_M5_HC | EVQLVESGGGLVQPGGSLSLACEVSGFILSDHYIDWVRQAPGKGLEWVGRS |
| | RNKAKRYTTEYAASVKGRFTISRDDSNNSLYLLMKSLKTEDTAVYFCTRAG |
| | YYDKNGDSLDALDIWGQGTMVTVSS (SEQ ID NO: 201) |
| DC2_M5_LC | DIQVTQSPSSLSASVGDRVTITCRTSQTVSSNLNWYQQRPGKAPKLLISGISD |
| | LHSGVPSRFSGSGSGTDSTLTISSLQPEDSATYYCQQSYSLPRTFGQGTKVEI |
| | K (SEQ ID NO: 202) |
| DC2_M17_HC | EVQLVESGSELKKPGASVRVSCEASGYSFTSHTIVWMRQAPGQGLECLGWI |
| | NTNTGNPTYAQGFTGRFVFSLDTSVSTAYLQISSLKAADTAVYYCARVLKC |
| | LGGSASCSGHGYYDYGMAVWGQGTTVTVSS (SEQ ID NO: 203) |
| DC2_M17_LC | DIVLTQSPLSLPVTPGEPASISCRSSQSLLHSNGYNFLHWYLQKPGQSPQLLI |
| | YLGSIRASGVPDRFSGSGSGTDFTLKISRVEAEDVGVYYCMQALQTFMYTF |
| | GQGTKLEIK (SEQ ID NO: 204) |
| DC2_M287_HC | EVQLVESGGGLVQPGGSLRLSCAASGFTFRDYWMIWVRQTPGKGLEWVA |
| | NINRNGNEKHYVDSLKGRFTISRDNTKNSLYLQVNGLSAEDTAVYYCVRDS |
| | SPSFGPGNYYDAFDIWGQGTTVTVSS (SEQ ID NO: 205) |
| DC2_M287_LC | DIRVTQSPSSLSASVGDRVTITCRASQDIRNEVGWYQQKPGQAPKVLIFAAS |
| | TLQSGVPSRFRGSGSGTVFTLTISSLQPEDLATYYCLQDFNYPRTFGQGTKV |
| | DIK (SEQ ID NO: 206) |
| DC2_M301_HC | QVQLVQSGAEVKKPGAAVKVSCKASGYTFSGYYIHWVRQAPGQGLEWMG |
| | WINPNSGGTDFAQKFQGRVTMTRDTSISTAYMELSRLRSDDTAVYLCSRDP |
| | GYTYGYPLGYWGQGTLVTVSS (SEQ ID NO: 207) |
| DC2_M301_LC | ETTLTQSPGTLSLSPGERATLSCRASQSVSSSYLAWYQQKPGQAPRLLIYGT |
| | SSRATGIPDRFSGRGSGTDFTLTISRLEPEDFAVYYCQQYGNSPPYTFGQGTK |
| | VDIK (SEQ ID NO: 208) |
| DC2_M336_HC | QVQLVQSGGRLVKPGGSLRLSCAASGFTIIDSYMSWIRQAPGKGLEWISYIN |
| | PSGSVIYYSDSVKGRFISSRDNAKNSVYLQLSSLRAEDAAVYYCARGIYDQS |
| | DAFDLWGQGTTVTVSS (SEQ ID NO: 209) |
| DC2_M336_LC | DIVLTQSPSSLSASVGDRVTITCRASQGISNSLAWYQQRPGKAPKLLIYGASR |
| | LGSGVPSRFSGSGSGADYTLTISGLQPEDFATYYCQQYYITPPMTFGPGTKV |
| | DIK (SEQ ID NO: 210) |
| DC2_M342_HC | QVQLVQSGAEVKKSGSSVTVSCKASGSTFISHAISWVRQAPGEGLEWMGRL |
| | IPVSGTPKYAHKFQGRLTITADDSTTTVYLTLRSLRFEDTAVYYCATWDAD |
| | STTLLYYFMDVWGKGTTVTVSS (SEQ ID NO: 211) |
| DC2_M342_LC | DIQVTQSPSTLSASVGDRVTITCRASQSISRWLAWYQQKPGKAPKLLIYKAS |
| | SLESGVPSRFSGTGSGTEFTLTISSLQPDDFATYYCQQYNSYSTWTFGQGTK |
| | LEIK (SEQ ID NO: 212) |

HC = Heavy chain; LC = Light chain

“And/or” as used herein, for example, with option A and/or option B, encompasses the separate embodiments of (i) option A, (ii) option B, and (iii) option A plus option B.

All combinations of the various elements described herein are within the scope of the invention unless otherwise indicated herein or otherwise clearly contradicted by context.

This invention may be better understood from the Experimental Details, which follow.

### EXEMPLIFICATIONS

### Example 1: Materials and Methods for Examples 2-9

Ethics Statement. The CHIKV convalescent donor DC2 was previously identified (25) and after informed written consent, blood samples were collected and PBMCs isolated by Ficoll gradient centrifugation. The study protocol was approved by the Institutional Review Board of the Albert Einstein College of Medicine (protocol IRB #2016-6137).

The mouse challenge studies were carried out in accordance with the recommendations in the Guide for the Care and Use of Laboratory Animals of the National Institutes of Health. The protocols were approved by the Institutional Animal Care and Use Committee at the Washington University School of Medicine (Assurance number A3381-01) under animal use approval number 20180234.

Cells and viruses. Vero cells were cultured in Dulbecco's Modified Eagle Medium (DMEM) supplemented with 10% fetal bovine serum at 5% CO2, 37° C. ExpiCHO-S cells (Gibco) were maintained in ExpiCHO expression media as per manufacturer's instructions. CHIKV 181/25 was obtained from Dr. Robert B. Tesh (University of Texas Medical Branch). The Mayaro Guyane virus (NR-49911) was obtained through BEI Resources as part of the World Reference Center for Emerging Viruses and Arboviruses (WRCEVA) program. The ONNV SG650 (62), RRV T48 (63), and MAYV TRVL-4675 (64) infectious clones were obtained from Andres Merits (University of Tartu). SFV and SINV were grown from infectious clones pSP6-SFV4(65) and dsTE12Q (66), respectively. Viruses were propagated in BHK-21 cells from Ari Helenius (ETH Zurich).

CHIKV and MAYV glycoprotein production. CHIKV p62-E1 and E1′ were expressed in Drosophila S2 cells and purified as previously described (25, 67). CHIKV E2 residues S1-Y361 (strain LR-2006) were codon-optimized, synthesized (Integrated DNA Technologies), and inserted into the pET21a vector. The plasmid construct was transformed into BL21 (DE3) competent cells (Thermo Fisher), grown to an optimal density (600 nm) of 0.8 and induced with 0.1 mM IPTG for 5 h. Cells were harvested, and inclusion bodies were isolated and refolded as previously described (67). CHIKV E2 protein was further purified by HiLoad 16/600 Superdex 75 size exclusion chromatography (GE Healthcare) and purity was assessed by SDS-PAGE. The MAYV p62-E1 insert was constructed in the same manner as CHIKV p62-E1 (23) based on the MAYV BeAr 20290 sequence. The sequence contained the native MAYV secretion signal, p62 ectodomain, Gly-Ser linker, E1 ectodomain, and a Strep II affinity tag. The sequence was codon-optimized, synthesized (IDT), and cloned into pT353 vector for S2 expression. Protein was purified by Strep-Tactin (IBA) affinity chromatography and purity was assessed by SDS-PAGE and size-exclusion chromatography.

Human mAb isolation. Isolation of human mAbs was performed as previously described (68). Antigen-reactive memory B cells were isolated from human PBMCs by fluorescence activated cell sorting (FACS) with the following antibodies: anti-human CD8 (PE-Cy7), CD3 (PE-Cy7), CD14 (PE-Cy7), CD20 (Pacific Blue), CD27(APC), IgG (FITC). MAYV p62-E1 was biotinylated using EZ-Link NHS-PEG4-Biotin (Life Technologies) and detected with streptavidin conjugated phycoerythrin (Life Technologies). Cells were sorted into single PCR tubes, and cDNA was generated by RT-PCR. Nested PCR was performed with IgH- and IgK-specific primers and cloned into pMAZ vector (69) for sequencing and recombinant expression. Sequences were analyzed using IMGT/V-quest tool (70). MAbs were transiently transfected in ExpiCHO cells as per the manufacturer's protocol (Gibco) and purified by Protein A chromatography. Fab constructs containing a C-terminal His tag on the heavy chain were transiently transfected in ExpiCHO cells and purified by Ni-NTA affinity chromatography.

ELISA binding assays. To assess mAb reactivity by ELISA, CHIKV and MAYV glycoproteins were coated on half-area 96-well high binding plates (Costar) at 200 ng/well. Wells were blocked with 1% BSA at 25° C. for 2 h and washed 5 times with PBS-T (PBS pH 7.4, 0.05% Tween-20). MAbs were diluted in PB-T (PBS pH 7.4, 0.2% BSA, 0.05% Tween) and incubated for 1 h at 37° C. Plates were washed and Protein A conjugated to HRP (Life Technologies) was added at a 1:2000 dilution. After 1 h incubation at 37° C., plates were washed and developed using TMB (Thermo Fisher). Absorbance at 450 nm was measured on Synergy H4 Hybrid reader (BioTek).

BLI binding assays. MAb binding kinetics to CHIKV and MAYV p62-E1 were measured by BLI using an OctetRed96 instrument (ForteBio). MAbs were immobilized on anti-human Fc capture sensors. Global data fitting to a 1:1 binding model was used to estimate values for the kon (association rate constant), koff (dissociation rate constant), and KD (equilibrium dissociation constant). Data were analyzed using ForteBio Data Analysis Software 9. For monovalent binding studies, Fabs were immobilized on anti-Penta-His sensors and analyzed as described above. For two-phase binning experiments, biotinylated MAYV p62-E1 was first loaded onto a streptavidin-coated sensor, and then the first mAb bound to saturation. The sensor was then added to a well containing the first and test mAbs at equimolar concentrations (50 nM).

Focus reduction neutralization assay. Vero cells were seeded at 2.5×104 cells/well and incubated for 24 h at 37° C. MAbs were serially diluted and incubated with 100-150 focus-forming units (FFU) of virus for 1 h at 37° C. Cells were inoculated with antibody-virus complexes for 1 h at 37° C. and cells were then overlaid with 1% carboxymethylcellulose in Modified Eagle Media (MEM), supplemented with 2% FBS and 10 mM HEPES pH 7.4. Plates were fixed 18 h post-infection with 1% PFA diluted in PBS and permeabilized in 1× PBS with 0.1% saponin and 0.1% BSA. Cells then were treated with primary antibody followed by horseradish peroxidase (HRP)-conjugated goat anti-mouse IgG and TrueBlue Peroxidase substrate (KPL). Developed foci where quantified on ImmunoSpot S6 Macroanalyzer (Cellular Technologies Ltd.). Infection of mAb-treated wells was determined relative to untreated control wells with virus alone. Non-linear regression analysis was performed using Prism 7 software (GraphPad Software, La Jolla CA).

Escape mutant generation. Vero cells were infected with rVSV-CHIKV in the presence of mAb at an IC90 concentration. Infection was monitored over days for cytopathic effects, and supernatants were harvested from the infected wells. After 4 passages under mAb selection, individual clones were plaque-purified and expanded. Viral RNA isolation was performed using a Viral RNA Kit (Zymo Research), and cDNA synthesis was performed. CHIKV glycoprotein genes were amplified by PCR and sequenced to analyze for the presence of mutations.

Neutralization of rVSV-CHIKV WT and escape mutants was tested by complexing serially diluted mAb with virus for 1 h at 37° C. prior to addition to cell monolayers in 96-well plates. Cells were fixed 9 h post-infection, washed with PBS, and treated with Hoescht stain. The relative infection was measured by automated counting of eGFP+ cells using a Cytation5 Imager (BioTek).

Mouse infection experiments. Four-week-old WT C57BL/6J male mice were purchased from Jackson Laboratories (000664). 200 μg (10 mg/kg) of mAbs DC2.M16, DC2.M357, or an isotype control were administered to individual mice by intraperitoneal injection 1 d before inoculation in the left footpad with 103 FFU of MAYV-BeH407 or CHIKV-La Reunion OPY1 in Hank's Balanced Salt Solution (HBSS) supplemented with 1% heat-inactivated FBS. Foot swelling was monitored via measurements (width×height) using digital calipers. At 3 days post-infection, tissues were harvested after extensive perfusion with 40 ml of PBS. Viral RNA was recovered and measured by qRT-PCR using a standard curve made by RNA isolated from a viral stock of known titer to determine FFU equivalents.

### Example 2: Generation and Characterization of Recombinant MAYV_p62-E1 Hybrid Protein

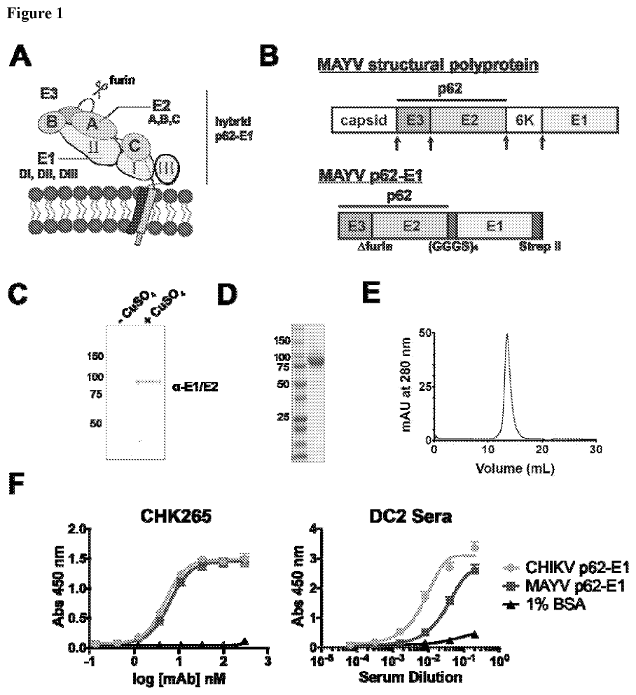

To assess the cross-reactivity of antibodies to the related alphavirus MAYV from patients who experienced heterologous CHIKV infections, a recombinant protein construct containing the MAYV E2 and E1 domains tethered by a flexible glycine-serine linker for heterologous expression in S2 Drosophila cells was designed (FIG. 1A), a strategy previously employed for the CHIKV glycoprotein (23). The MAYV ectodomain, or MAYV “p62-E1” also contains E3 due to introduction of a mutation at the furin cleavage site (FIG. 1B), which prevents maturation. Inducible expression of MAYV p62-E1 was confirmed by immunoblotting (FIG. 1C), and Strep-Tactin affinity purification yielded 1-2 mg/L of the glycoprotein at >90% purity as determined by SDS-PAGE and size-exclusion chromatography (FIG. 1D and 1E). The reactivity of CHK-265, a previously described murine cross-reactive alphavirus mAb (38), was tested against both MAYV and CHIKV p62-E1 glycoproteins by ELISA (FIG. 1F). CHK-265 showed similar binding to both MAYV and CHIKV antigens, consistent with previous work (38) and confirming the MAYV p62-E1 adopts an antigenically relevant conformation. Serum from a CHIKV convalescent donor “DC2” was tested by ELISA for reactivity against MAYV p62-E1 (FIG. 1F). Donor DC2 was exposed to CHIKV in the Dominican Republic, and exhibited symptoms of fever, myalgia, and persistent arthralgia (25). Notably, MAYV has not been reported to circulate in the Dominican Republic. Serum from donor DC2 reacted with both CHIKV and MAYV glycoproteins, suggesting the presence of anti-MAYV antibodies. Together, the efficient production and antigenic analysis of recombinant MAYV p62-E1 provided a rationale for its use as a probe to isolate and characterize cross-reactive alphavirus mAbs.

### Example 3: Isolation of Cross-Reactive MAYV mAbs by Single B-Cell Cloning

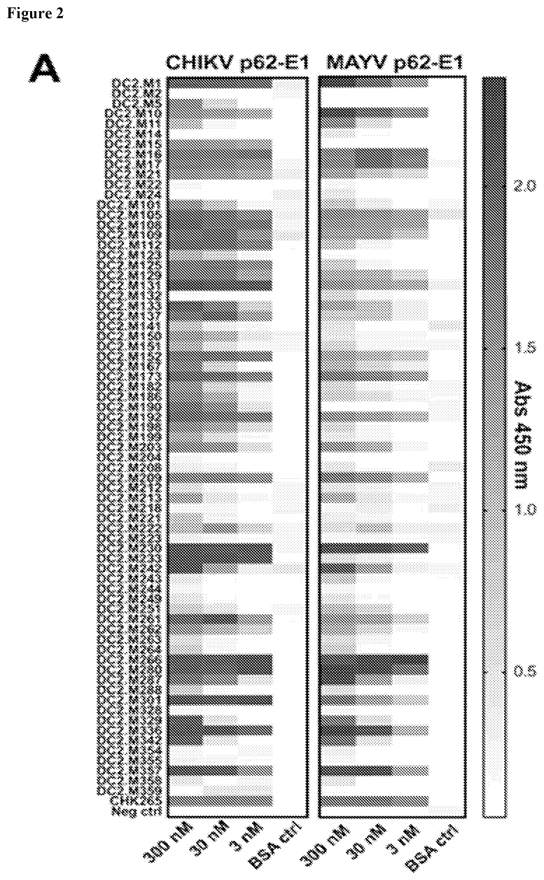

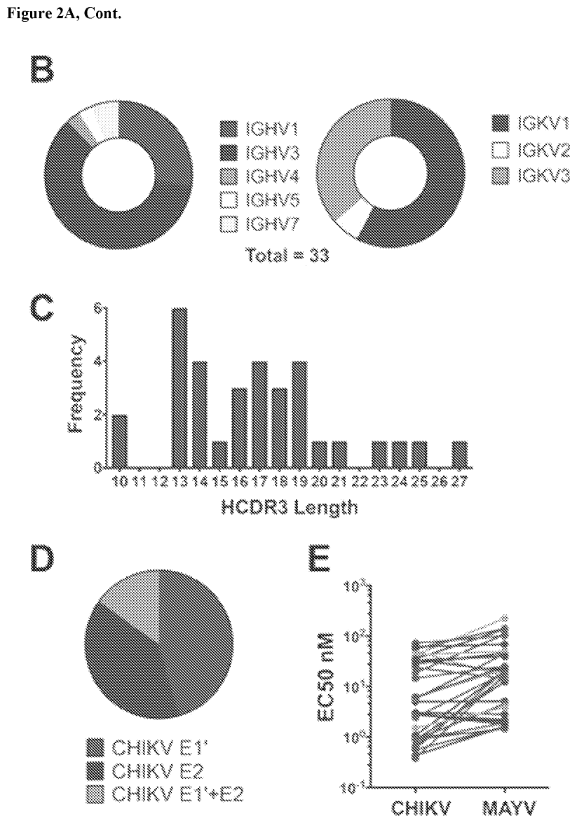

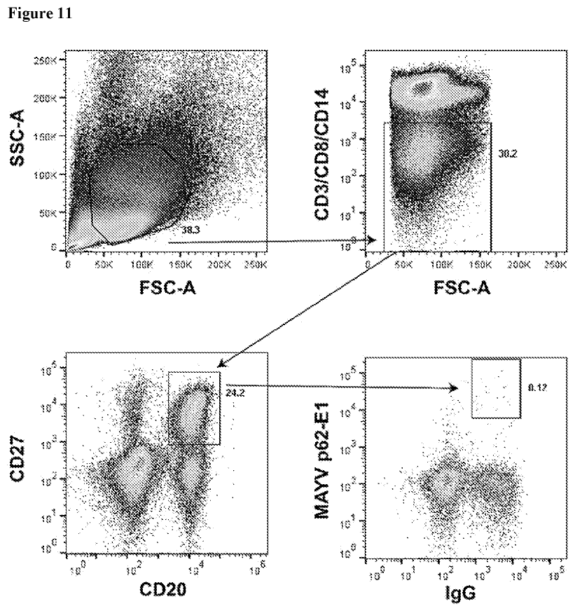

To isolate cross-reactive alphavirus mAbs from donor DC2, a single B cell cloning strategy using MAYV p62-E1 as a heterologous sorting antigen was employed (FIG. 11). Individual CD20⁺ CD27⁺ IgG⁺ MAYV p62-E1⁺ memory B cells were sorted, and then a panel of 71 mAbs were cloned and recombinantly expressed from the corresponding cDNA. IGKV light chains were focused exclusively on since they generally have favorable stability and expression profiles (39). These mAbs were assessed for reactivity to CHIKV and MAYV p62-E1 by ELISA using three different concentrations (3, 30, and 300 nM) of mAb (FIG. 2A). The majority of the mAbs tested (58 of 71) bound to both the CHIKV and MAYV p62-E1 proteins. Thirty-three mAbs (termed DC2.Mx) showed strong cross-reactivity at 30 nM to both CHIKV and MAYV glycoproteins and were characterized further. Sequence analysis of the cross-reactive mAbs showed a diverse distribution of V-gene usage for both IGHV and IGKV (FIG. 2B) as well as a range of HCDR3 lengths (FIG. 2C). To determine whether cross-reactive mAbs targeted the E1 or E2 subunit, ELISA reactivity to the ectodomains of the two independent CHIKV glycoprotein subunits (E1′ or E2) was assessed (FIG. 2D). There were approximately equal numbers of cross-reactive mAbs targeting E1′ and E2. The half maximal effective concentration (EC₅₀) values for binding to CHIKV and MAYV p62-E1 ranged from 0.5-72 nM and 1.6-220 nM, respectively (FIG. 2E and Table 3). Thus, a substantial subset of mAbs isolated from a CHIKV convalescent donor cross-reacted with MAYV, and these mAbs targeted epitopes on both the E1 and E2 subunits.

TABLE 3
Cross-reactive human mAb sequence and reactivity profiles

| | | | | SEQ | | | CHKV | MAYV |
|---|---|---|---|---|---|---|---|---|
| | | CDR | | ID | | | p62-E1 | p62-E1 |
| mAb ID | V-gene | length | CDR3 | NO: | E1′ | E2 | EC₅₀ nM | EC₅₀ nM |
| DC2.M1_H | IGHV3- | 8.8.14 | ARPFSMSWFEGFEF | 45 | + | − | 0.38 | 17 |
| | 30 | | | | | | | |
| DC2.M1_L | IGKV1- | 6.3.9 | QQSYTAPLT | 48 | | | | |
| | 39 | | | | | | | |
| DC2.M5_H | IGHV3- | 8.10.18 | TRAGYYDKNGDSLDALDI | 9 | − | + | 33 | 39 |
| | 72 | | | | | | | |
| DC2.M5_L | IGKV1- | 6.3.9 | QQSYSLPRT | 12 | | | | |
| | 39 | | | | | | | |
| DC2.M10_H | IGHV1- | 8.8.17 | ARDHLNRDSSSRGFMDY | 51 | + | − | 2.5 | 18 |
| | 46 | | | | | | | |
| DC2.M10_L | IGKV3- | 6.3.9 | QQRGTWPPS | 54 | | | | |
| | 11 | | | | | | | |
| DC2.M11_H | IGHV3- | 8.8.20 | ARASGWQRTGTKYYYYGM | 57 | − | + | 34 | 21 |
| | 30 | | DV | | | | | |
| DC2.M11_L | IGKV1- | 6.3.9 | LQYDNLPYS | 60 | | | | |
| | 33 | | | | | | | |
| DC2.M16_H | IGHV3- | 8.8.13 | ARGIYYQSDAFDI | 63 | − | + | 2.8 | 1.8 |
| | 11 | | | | | | | |
| DC2.M16_L | IGKV1- | 6.3.10 | QQYYSTPPMT | 66 | | | | |
| | NL1 | | | | | | | |
| DC2.M17_H | IGHV7- | 8.8.25 | ARVLKCLGGSASCSGHGY | 15 | + | − | 0.9 | 1.9 |
| | 4 | | YDYGMAV | | | | | |
| DC2.M17_L | IGKV2- | 11.3.10 | MQALQTFMYT | 18 | | | | |
| | 28 | | | | | | | |
| DC2.M21_H | IGHV1- | 8.8.17 | ARDHLNRDSTSRGFIDS | 69 | + | + | 43 | 50 |
| | 46 | | | | | | | |
| DC2.M21_L | IGKV3- | 6.3.9 | EQRGDWPLT | 72 | | | | |
| | 11 | | | | | | | |
| DC2.M101_H | IGHV7- | 8.8.16 | ARGRGATTVTTYYFDY | 75 | + | − | 6.2 | 25 |
| | 4 | | | | | | | |
| DC2.M101_L | IGKV3- | 6.3.10 | QHYINRPGRT | 78 | | | | |
| | 15 | | | | | | | |
| DC2.M105_H | IGHV1- | 8.8.24 | ARTVYVDKGMVMVRRLYQ | 81 | + | − | 0.56 | 1.6 |
| | 2 | | YFGMDV | | | | | |
| DC2.M105_L | IGKV3- | 7.3.10 | QQYGISPEFT | 84 | | | | |
| | 20 | | | | | | | |
| DC2.M108_H | IGHV3- | 8.8.13 | ARGLYIQSDAFDL | 87 | − | + | 2.9 | 2.0 |
| | 11 | | | | | | | |
| DC2.M108_L | IGKV1- | 6.3.10 | QQYYNTPPIT | 90 | | | | |
| | NL1 | | | | | | | |
| DC2.M109_H | IGHV5- | 8.8.27 | ARRPREQLGRLLLGDVVPH | 93 | + | + | 1.5 | 3.8 |
| | 51 | | GRNDAFDI | | | | | |
| DC2.M109_L | IGKV1- | 6.3.9 | QQSYGTLWT | 96 | | | | |
| | 39 | | | | | | | |
| DC2.M112_H | IGHV3- | 8.8.18 | ARDEAVGPYQYAAEYFHH | 99 | + | − | 0.58 | 21 |
| | 33 | | | | | | | |
| DC2.M112_L | IGKV3- | 6.3.8 | QQYNNWLT | 102 | | | | |
| | 15 | | | | | | | |
| DC2.M125_H | IGHV3- | 8.8.13 | VRGVYVQSDAFDI | 105 | − | + | 28 | 46 |
| | 48 | | | | | | | |
| DC2.M125_L | IGKV1- | 6.3.10 | QQYYSTPPIT | 108 | | | | |
| | NL1 | | | | | | | |
| DC2.M129_H | IGHV3- | 8.8.10 | VRDKDGVFDY | 111 | + | + | 6.0 | 22 |
| | 11 | | | | | | | |
| DC2.M129_L | IGKV1- | 6.3.10 | QQYNTYPHST | 114 | | | | |
| | 5 | | | | | | | |
| DC2.M131_H | IGHV3- | 8.8.13 | ARGIYHQSDAFDI | 117 | − | + | 2.7 | 2.2 |
| | 11 | | | | | | | |
| DC2.M131_I | IGKV1- | 6.3.10 | QQYYSTPPIT | 108 | | | | |
| | NL1 | | | | | | | |
| DC2.M133_H | IGHV3- | 8.8.19 | VRDNSPSFGPGNYYDAFDI | 123 | − | + | 61 | 68 |
| | 7 | | | | | | | |
| DC2.M133_L | IGKV1- | 6.3.9 | LQDYNYPRT | 126 | | | | |
| | 6 | | | | | | | |
| DC2.M152_H | IGHV1- | 8.8.19 | TRDNYNSWRGPDFYTGVD | 129 | + | − | 19 | 110 |
| | 2 | | V | | | | | |
| DC2.M152_L | IGKV3- | 6.3.9 | QQRSSLGLS | 132 | | | | |
| | 11 | | | | | | | |
| DC2.M173_H | IGHV1- | 8.8.16 | ARDACSGGSCYTPFDY | 135 | + | − | 5.7 | 45 |
| | 46 | | | | | | | |
| DC2.M173_L | IGKV3- | 6.3.9 | QQYNNWPRT | 138 | | | | |
| | 15 | | | | | | | |
| DC2.M192_H | IGHV3- | 8.8.19 | ARRGFHYDSSGYYYYGMD | 141 | + | − | 0.67 | 16 |
| | 33 | | V | | | | | |
| DC2.M192_L | IGKV2- | 11.3.10 | MQALQTPPFT | 144 | | | | |
| | 28 | | | | | | | |
| DC2.M203_H | IGHV3- | 8.7.16 | VRLGGYIGNDRDAFDI | 147 | − | + | 0.44 | 1.60 |
| | 13 | | | | | | | |
| DC2.M203_L | IGKV1- | 6.3.10 | QQYNTYPHST | 114 | | | | |
| | 5 | | | | | | | |
| DC2.M209_H | IGHV3- | 8.10.18 | TREGVVVAARYYYYIMDV | 153 | + | + | 50 | 16 |
| | 49 | | | | | | | |
| DC2.M209_L | IGKV1- | 6.3.9 | QQNYRTPRT | 156 | | | | |
| | 39 | | | | | | | |
| DC2.M222_H | IGHV1- | 8.8.17 | ARDLEAANSVILPRLFY | 159 | + | − | 24 | 15 |
| | 69 | | | | | | | |
| DC2.M222_L | IGKV1- | 6.3.10 | QQYYSTPPIT | 108 | | | | |
| | NL1 | | | | | | | |
| DC2.M230_H | IGHV3- | 8.8.13 | ARGIYYQSDAFDT | 165 | − | + | 3.0 | 2.8 |
| | 11 | | | | | | | |
| DC2.M230_L | IGKV1- | 6.3.10 | QQYYSTPPIT | 108 | | | | |
| | NL1 | | | | | | | |
| DC2.M242_H | IGHV1- | 8.8.14 | ARDIAPTGAWWFDS | 171 | + | − | 72 | 100 |
| | 2 | | | | | | | |
| DC2.M242_L | IGKV3- | 7.3.8 | QQYGSPRT | 174 | | | | |
| | 20 | | | | | | | |
| DC2.M261_H | IGHV3- | 8.8.10 | VRWVAYYFDN | 177 | + | − | 15 | 25 |
| | 30 | | | | | | | |
| DC2.M261_L | IGKV3- | 7.3.10 | QYYGSLPPIT | 180 | | | | |
| | 20 | | | | | | | |
| DC2.M262_H | IGHV4- | 8.7.14 | ARLGYSHPYWYFDL | 183 | + | − | 56 | 140 |
| | 59 | | | | | | | |
| DC2.M262_L | IGKV1- | 6.3.10 | QQSYSPPLIT | 186 | | | | |
| | 39 | | | | | | | |
| DC2.M266_H | IGHV3- | 8.8.15 | ARVQGEQWRGLHFDS | 189 | + | − | 0.89 | 1.5 |
| | 11 | | | | | | | |
| DC2.M266_L | IGKV3- | 6.3.8 | QHRSNWPA | 192 | | | | |
| | 11 | | | | | | | |
| DC2.M280_H | IGHV3- | 8.8.23 | AKDSGWLRRGDYDTSGFY | 195 | − | + | 1.1 | 21 |
| | 9 | | GPIDY | | | | | |
| DC2.M280_L | IGKV1- | 6.3.9 | QQSYGTLLT | 198 | | | | |
| | 39 | | | | | | | |
| DC2.M287_H | IGHV3- | 8.8.19 | VRDSSPSFGPGNYYDAFDI | 21 | + | + | 43 | 220 |
| | 7 | | | | | | | |
| DC2.M287_L | IGKV1- | 6.3.9 | LQDFNYPRT | 24 | | | | |
| | 6 | | | | | | | |
| DC2.M301_H | IGHV1- | 8.8.14 | SRDPGYTYGYPLGY | 27 | + | − | 0.88 | 4.6 |
| | 2 | | | | | | | |
| DC2.M301 L | IGKV3- | 7.3.10 | QQYGNSPPYT | 30 | | | | |
| | 20 | | | | | | | |
| DC2.M336_H | IGHV3- | 8.8.13 | ARGIYDQSDAFDL | 33 | − | + | 0.90 | 12.5 |
| | 11 | | | | | | | |
| DC2.M336_L | IGKV1- | 6.3.10 | QQYYITPPMT | 36 | | | | |
| | NL1 | | | | | | | |
| DC2.M342_H | IGHV1- | 8.8.17 | ATWDADSTTLLYYFMDV | 39 | − | + | 35 | 130 |
| | 69 | | | | | | | |
| DC2.M342_L | IGKV1- | 6.3.10 | QQYNSYSTWT | 42 | | | | |
| | 5 | | | | | | | |
| DC2.M357_H | IGHV3- | 8.9.20 | ARDRYYYDSSAYFLIDAFDI | 214 | − | + | 4.9 | 5.0 |
| | 30 | | | | | | | |
| DC2.M357_L | IGKV3- | 6.3.10 | QQRSNWPPIT | 6 | | | | |
| | 11 | | | | | | | |

### Example 4: Cross-Reactive Human mAbs Exhibit a Range of Neutralizing Potencies and Breadth

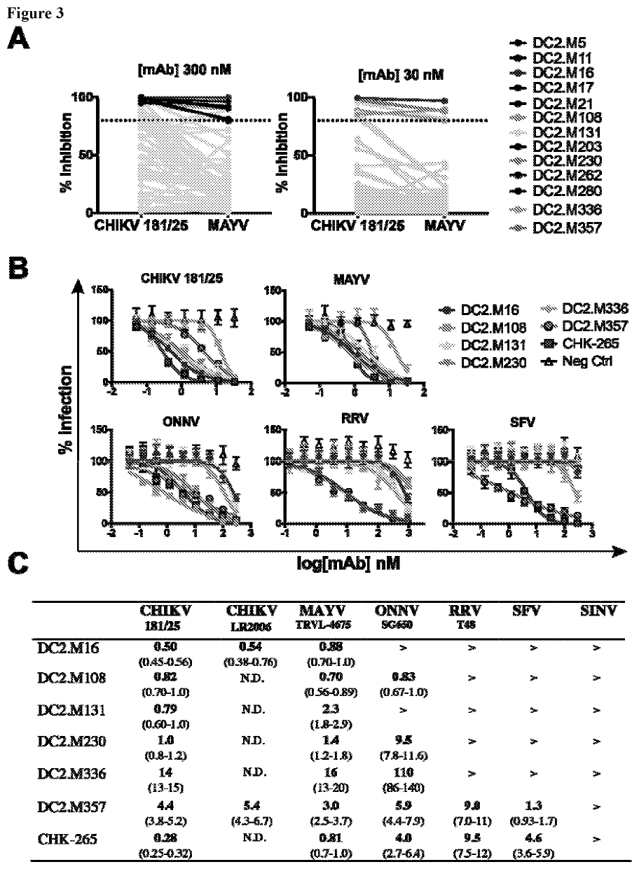

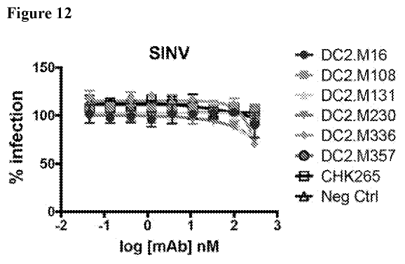

Neutralizing activity of all 71 mAbs was tested by focus reduction neutralization test (FRNT) against CHIKV 181/25 and MAYV at two mAb concentrations (30 and 300 nM) (FIG. 3A). While many mAbs neutralized CHIKV 181/25 infection at both concentrations, only six (DC2.M16, DC2.M108, DC2.M131, DC2.M230, DC2.M336, and DC2.M357) inhibited CHIKV 181/25 and MAYV at both 300 nM and 30 nM concentrations. These neutralizing mAbs were all E2-reactive, and none of the E1-reactive mAbs exhibited neutralizing activity. Full neutralization curves revealed IC₅₀ values ranging from 0.5-14 nM and 0.7-16 nM for CHIKV 181/25 and MAYV, respectively (FIGS. 3B and 3C). Next, the breadth of neutralization towards related arthritogenic alphaviruses ONNV, RRV, and Semliki Forest virus (SFV) was determined by FRNT (FIGS. 3B and 3C). Cross-neutralizing mAbs showed a range of inhibition against the different alphaviruses tested. DC2.M16, DC2.M131 and DC2.M336 exhibited little neutralizing activity against ONNV, RRV or SFV, whereas DC2.M108, DC2.M230 and DC2.M357 all neutralized ONNV, the closest genetic relative to CHIKV. Furthermore, DC2.M357 also neutralized RRV and SFV, with IC₅₀ values comparable to murine CHK-265. None of the mAbs tested neutralized the more distantly related SINV (FIG. 12). Additionally, IC₅₀ values for DC2.M16 and DC2.M357 against the pathogenic East/Central/South African CHIKV-2006 LR-OPY1 strain were comparable to those derived against the attenuated CHIKV 181/25 Asian strain (FIG. 3C).

### Example 5: Differential Binding Kinetics of Neutralizing and Non-Neutralizing mAbs

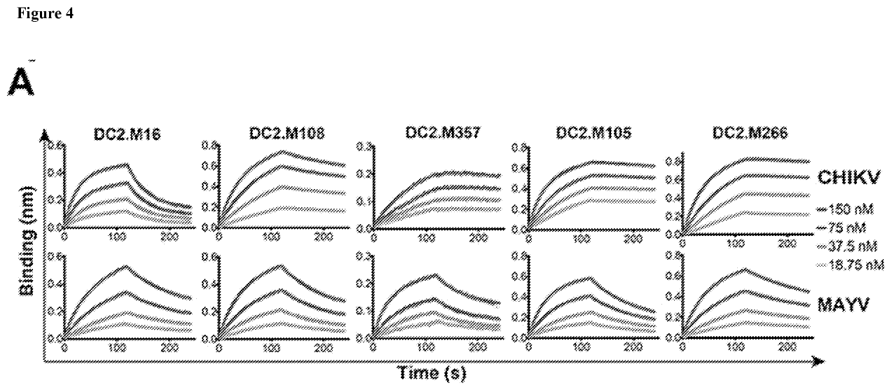

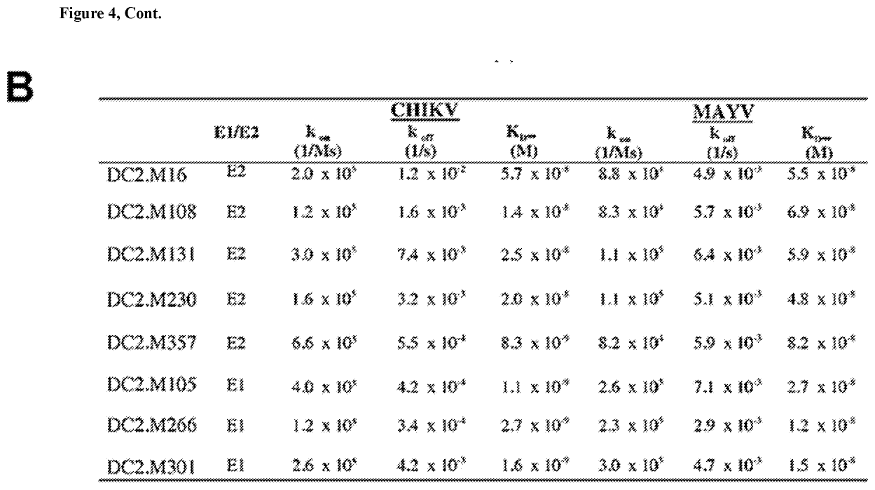

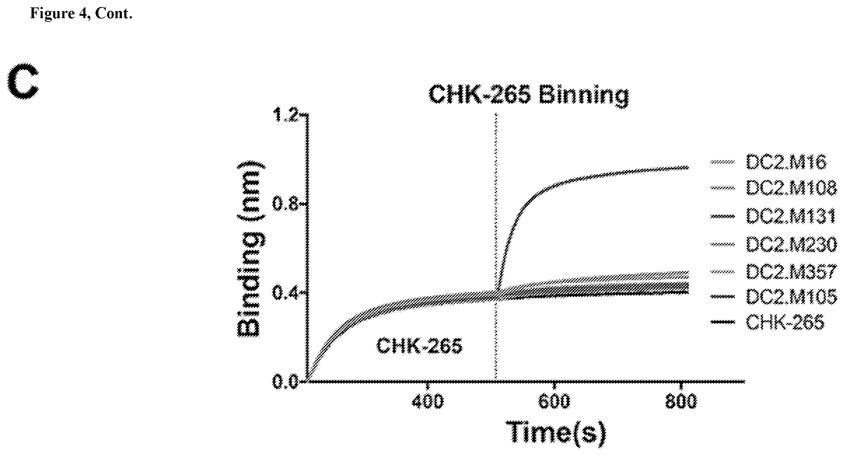

To evaluate the binding kinetics of the most potent human bNAbs for CHIKV and MAYV p62-E1 glycoproteins, biolayer interferometry (BLI) was used (FIG. 4A). MAbs were captured on an anti-human Fc sensor and then dipped into solutions of CHIKV or MAYV p62-E1. While most sensorgrams could be fit with a 1:1 binding model, since the IgG is bivalent, avidity effects cannot be ruled out and therefore dissociation constants derived from on and off rates (kon and koff) have been termed as “apparent” (KDapp). The five E2-directed bNAbs DC2.M16, M108, M131, M230, and M357 had KDapp values ranging from 8-57 nM and 48-82 nM for CHIKV and MAYV p62-E1, respectively (FIG. 4B). Additionally, three E1-specific mAbs (DC2.M105, M266, and M301) were tested and all showed substantially higher affinities for both glycoproteins (1.0-2.7 nM for CHIKV p62-E1 and 12-27 nM for MAYV p62-E1) than the E2 mAbs. In all cases, the measured binding affinities are higher than the neutralizing IC₅₀ values, possibly due to avidity differences of mAb engagement on the monomeric recombinant glycoprotein versus the surface of the viral particle or the stoichiometry of binding required for neutralization (40). Notably, four of the eight mAbs tested (E2 mAb DC2.M357, and E1 mAbs DC2.M105, DC2.M266, and DC2.M301) had higher (˜10-fold) binding affinity for CHIKV p62-E1 than MAYV p62-E1. This enhanced binding to CHIKV p62-E1 was driven primarily by a lower off rate (koff). Thus, a range of kinetic binding profiles of E2-reactive bNAbs and non-neutralizing, cross-reactive E1-directed mAbs was observed against CHIKV and the heterologous MAYV antigen.

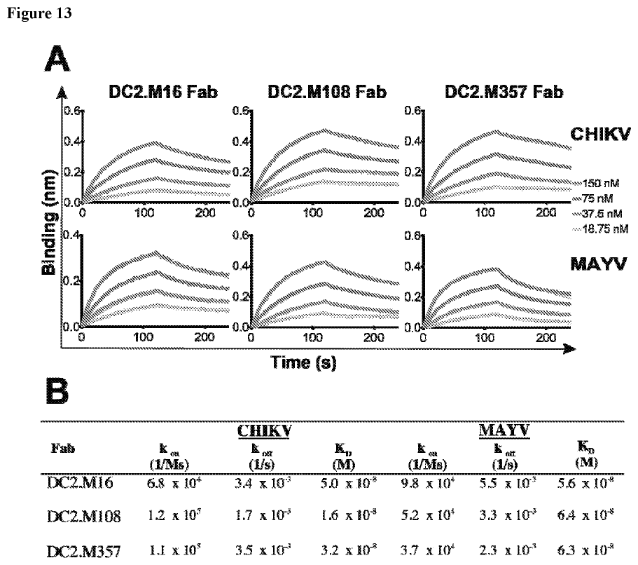

To determine the kinetics of monovalent binding, antigen-binding fragments (Fabs) of bNAbs DC2.M16, DC2.M108, and DC2.M357 were generated and tested interaction with CHIKV and MAYV glycoproteins (FIG. 13). The binding kinetics observed for the Fabs were largely consistent with the full-length mAbs, with K_{D} values ranging from 16 to 50 nM for CHIKV p62-E1 and 56 to 64 nM for MAYV p62-E1.

### Example 6: Cross-Neutralizing mAbs Target the B Domain of E2

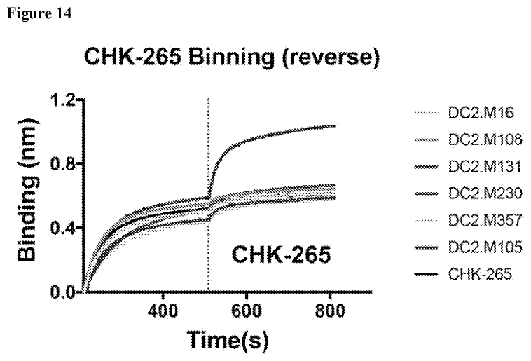

The epitope of murine CHK-265 lies primarily within the B domain of E2. To determine whether human bNAbs also target the B domain, two-phase epitope binning was performed (FIGS. 4C and 14). CHK-265 was loaded onto sensors coated with MAYV p62-E1 and then added to an equimolar mixture of CHK-265 and the human mAb. All cross-neutralizing mAbs tested showed minimal binding to the MAYV glycoprotein pre-loaded with CHK-265, indicating that they also target the B domain. In contrast, DC2.M105 (an E1-targeting mAb) bound the CHK-265/p62-E1 complex since their two epitopes are spatially distant.

### Example 7: Viral Escape Mutants of Cross-Neutralizing mAbs Map to Distinct Regions of the E2 B Domain

The cross-neutralizing human mAbs have similar binding affinities to recombinant MAYV and CHIKV glycoproteins and compete with B-domain specific CHK-265 but exhibited a range of potency and breadth against related alphaviruses. These observations suggest that subtle differences in their epitopes within the B domain may determine their functional activity. To test this hypothesis, viral escape mutants against DC2.M108 and DC2.M357 were generated using a replication-competent recombinant vesicular stomatitis virus bearing the CHIKV glycoproteins (rVSV-CHIKV) as previously described (25). DC2.M108 neutralizes CHIKV, ONNV, and MAYV, whereas DC2.M357 neutralizes these viruses as well as RRV and SFV.

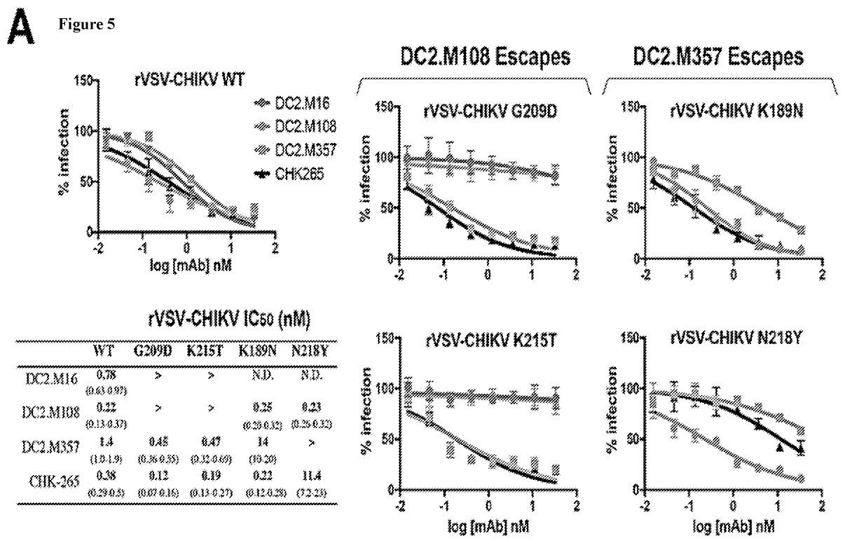

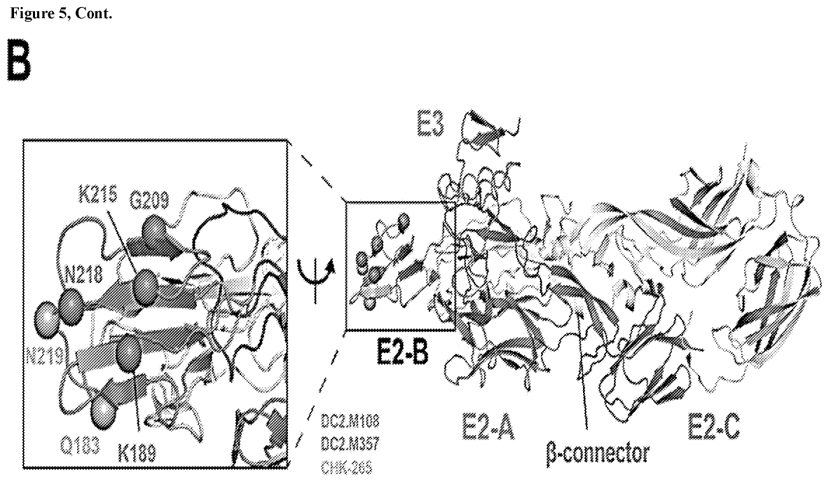

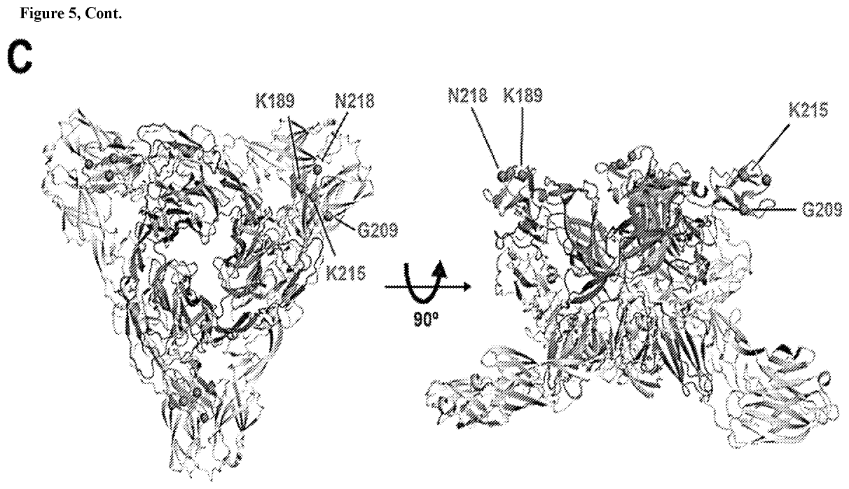

Both DC2.M108 and DC2.M357 efficiently neutralized rVSV-CHIKV (FIG. 5A). Serial passage of rVSV-CHIKV in the presence of these mAbs resulted in a resistant population from which plaques were selected, genotyped, and tested for neutralization. For both DC2.M108 and DC2.M357, two distinct viral escape mutants harboring a single point mutation were isolated. All mutations mapped to the B domain of E2, consistent with binning experiments with CHK-265. For DC2.M108, escape mutants G209D or K215T resulted in complete escape from DC2.M108 as well as the related mAb DC2.M16. However, these mutations did not confer escape for either DC2.M357 or CHK-265. For DC2.M357, the observed viral escape mutant K189N was only partially neutralized by DC2.M357 at high concentrations but was effectively neutralized by both DC2.M108 and CHK-265. Finally, the N218Y mutation was a complete escape from both DC2.M357 and CHK-265, but not DC2.M108.

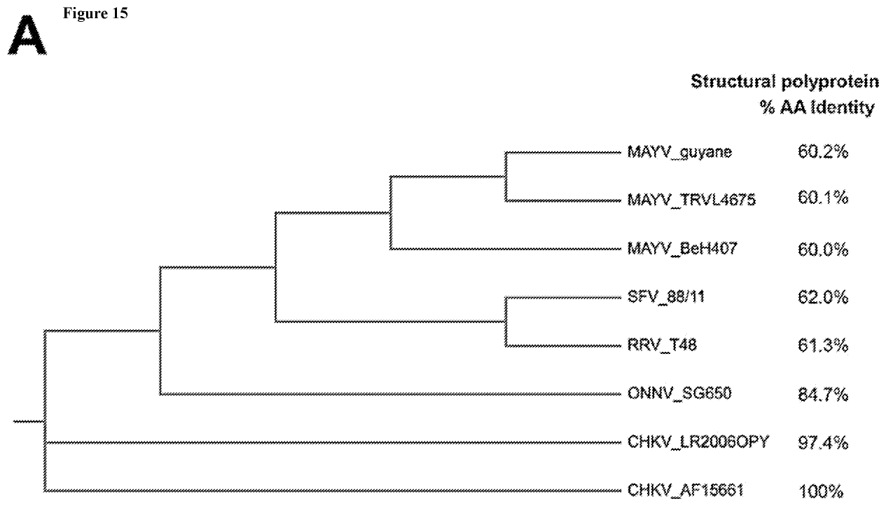

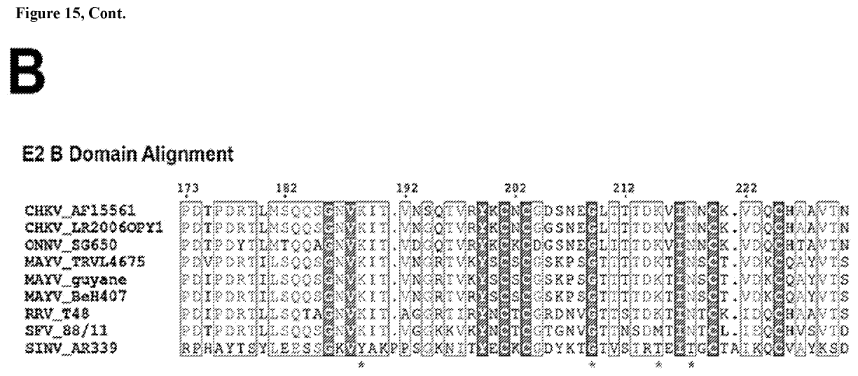

Mapping of these mutations to the CHIKV p62-E1 crystal structure (FIG. 5B), and comparison with the recently described CHK-265 viral escape mutations against RRV, revealed distinct regions within the β-sheet structure of the B domain that are targeted by cross-neutralizing mAbs. All mutations identified map to positions that are highly conserved amongst related alphaviruses (FIG. 15). Notably, the DC2.M357 escape positions K189 and N218 are more proximal to those previously described for CHK-265 (Q183 and N219), and these mAbs share similar neutralization breadth. The DC2.M108 escape positions G209 and K215 are more distal to the CHK-265 escape mutations, which may suggest a different angle of approach for binding of the DC2.M108 variable domains. Since some B domain mAbs can engage adjacent heterodimers on the alphavirus surface (38), the mutations were also mapped to the CHIKV trimeric spike (FIG. 5C). The DC2.M357 (K189 and N218) escape mutations are oriented toward the crest of the crown formed by the three E2 subunits on the prefusion trimer, whereas those for DC2.M108 are more lateral. Together, these data suggest that recognition of distinct residues within the B domain of E2 can determine the breadth of neutralization of related arthritogenic alphaviruses.

### Example 8: Germline Sequence and Mutagenesis Analysis of Cross-Neutralizing mAbs

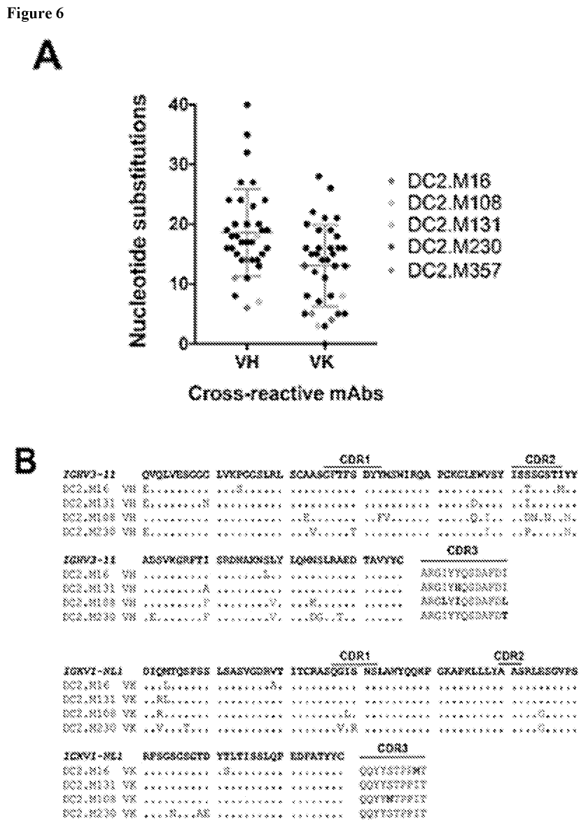

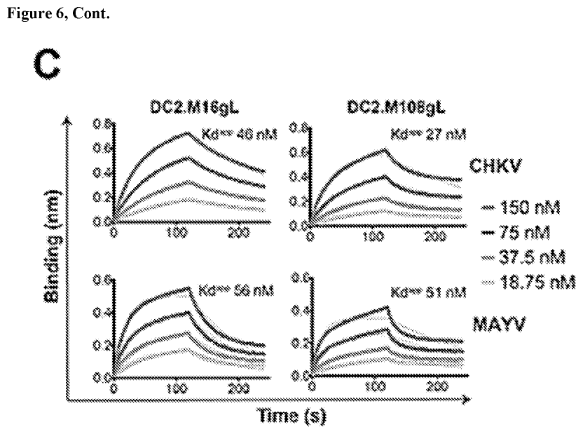

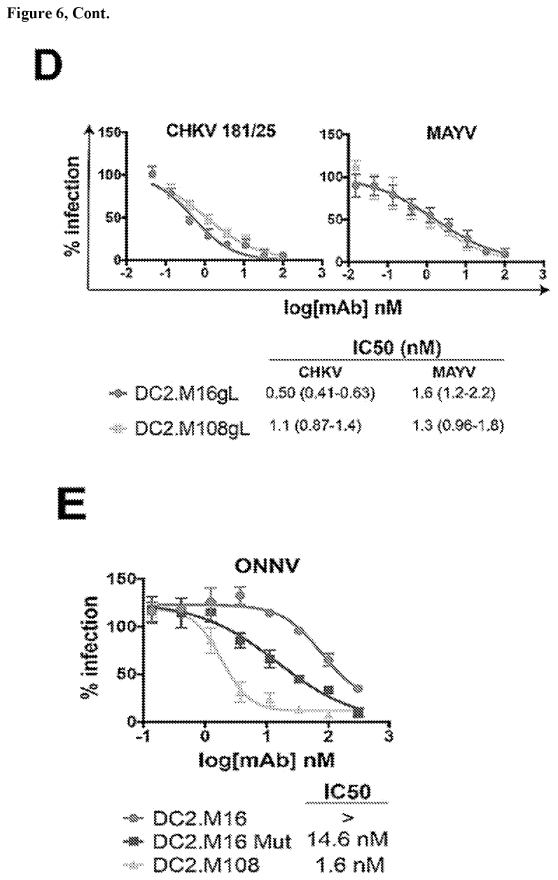

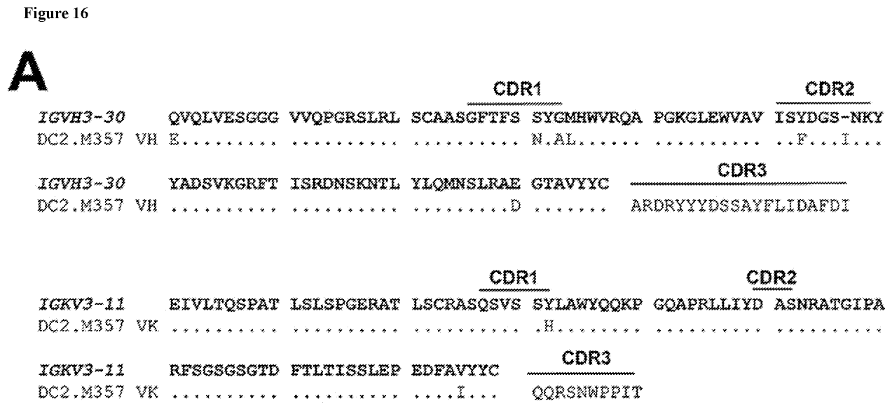

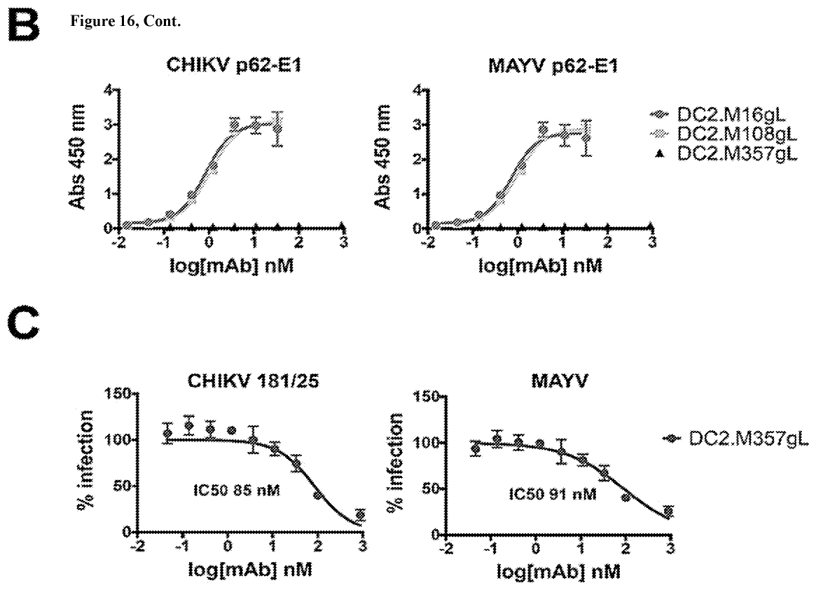

Sequence analysis of V-gene nucleotide substitutions showed that cross-reactive mAbs had a range of somatic mutations within the VH and VK domains (FIG. 6A). The most potent cross-neutralizing mAbs had relatively few substitutions and had the least divergence from germline. In particular, DC2.M16, DC2.M131, and DC2.M357 all shared >96% and >98% nucleotide identity to their IGHV and IGKV germlines, respectively. MAbs DC2.M16, DC2.M131, DC2.M108 and DC2.M230 all shared the same V-gene families IGHV3-11 and IGKV1-NL1, the same CDR lengths, the same J-gene family IGHJ3 and IGKJ3, and had similar sequences (FIG. 6B). This analysis suggests that all four of these mAbs were derived from a common progenitor. DC2.M357 has an unrelated sequence derived from IGHV3-30 and IGKV3-11 (FIG. 16). To determine the impact of V-gene somatic mutations on mAb function, inferred germline-revertants (gL) DC2.M16gL, DC2.M108gL and DC2.M357gL were generated, which harbored the V-gene germline sequence while retaining the junctional diversity at the CDR3s of the heavy and light chains. DC2.M16gL and DC2.M108gL bound CHIKV and MAYV p62-E1 with similar affinities to the somatically mutated, wild-type mAbs (FIG. 6C). Furthermore, DC2.M16gL and DC2.M108gL neutralized both CHIKV 181/25 and MAYV infection with equivalent potency to the wild-type mAbs (FIG. 6D). In contrast, DC2.M357gL did not bind to recombinant alphavirus glycoproteins by ELISA (FIG. 16) and neutralized CHIKV 181/25 and MAYV infection with a ˜20-fold lower potency than the wild-type mAb. These results demonstrate that human mAbs from IGHV3-11 and IGKV1-NL1 lineages can possess broad alphavirus recognition and neutralizing capacity from junctional diversity alone and not V-gene somatic mutations. In the case of DC2.M357, nine total amino acid mutations in V-gene IGHV3-30 and IGKV3-11, primarily within the CDR1 and CDR2, are critical for enhancing antigen-binding and neutralizing activity (FIG. 16). These data demonstrate that germline-like human mAbs harboring few somatic mutations can potently neutralize multiple alphaviruses.

Differences in ONNV neutralization by the IGHV3-11/IGKV1-NL1 mAbs were observed even though they share similar sequences. Notably, DC2.M108 and DC2.M230, but not DC2.M16 and DC2.131, neutralized ONNV and shared five mutations from the germline primarily in framework regions 2 and 3 proximal to HCDR2 (FIGS. 3B-3C and 6B). Substitution of the mutations shared by DC2.M108 and DC2.M230 (FIG. 6B) into the corresponding positions in the DC2.M16 sequence (DC2.M16 Mut) substantially improved neutralization of ONNV (FIG. 6E). Thus, the functional paratope permitting ONNV neutralization lies at least in part in these regions, and the neutralizing breadth of IGHV3-11/IGKV1-NL1 mAbs can be modulated by limited V-gene mutation.

### Example 9: Protective Efficacy of Cross-Neutralizing mAbs In Vivo

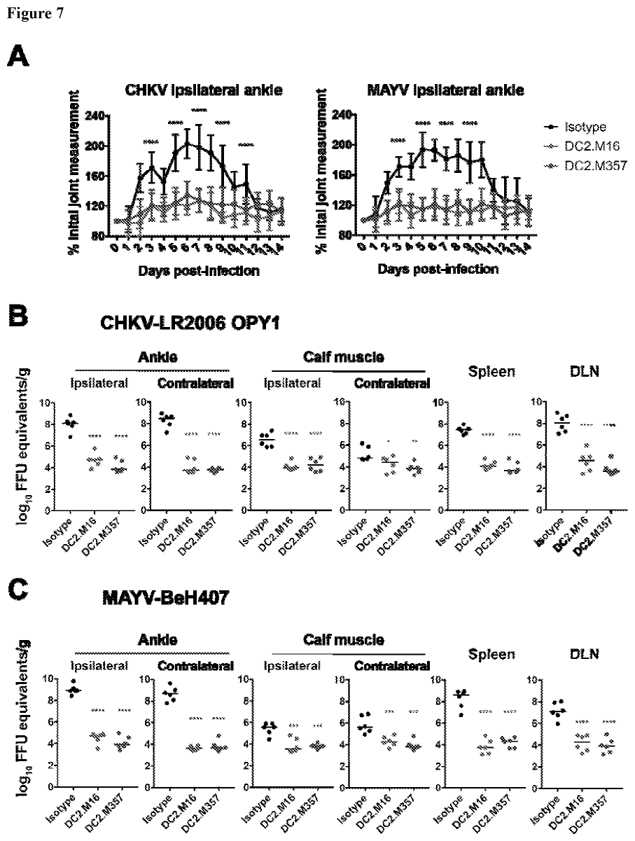

To determine whether human cross-neutralizing mAbs could confer protection against viral challenge in vivo, the ability of DC2.M16 and DC2.M357 mAb treatment to mitigate joint swelling and infection induced by CHIKV and MAYV was tested. Four-week old C57BL/6J mice were inoculated subcutaneously in the footpad with either CHIKV-LR2006 OPY1 or MAYV-BeH407 one day following intraperitoneal mAb administration. Joint swelling was measured daily for 14 days after inoculation (FIG. 7A). Both DC2.M16 and DC2.M357 reduced CHIKV and MAYV-induced joint swelling compared to an isotype control mAb.

To test the effect of mAb treatment on viral dissemination, mice were inoculated with CHIKV or MAYV one day after mAb administration, and viral RNA was measured by qRT-PCR in ankle, calf muscle, spleen, and draining lymph node tissues at 3 days after infection (FIGS. 7B and 7C). CHIKV and MAYV viral RNA levels in both the ipsilateral and contralateral ankle joint and calf muscle were reduced markedly in mice treated with either DC2.M16 or DC2.M357 compared to the isotype control mAb. In addition, mice treated with DC2.M16 or DC2.M357 had decreased viral RNA in the spleen and draining lymph node. These data demonstrate that DC2.M16 and DC2.M357, two near-germline human mAbs from distinct lineages, can protect against musculoskeletal infection and disease caused by CHIKV and MAYV in mice.

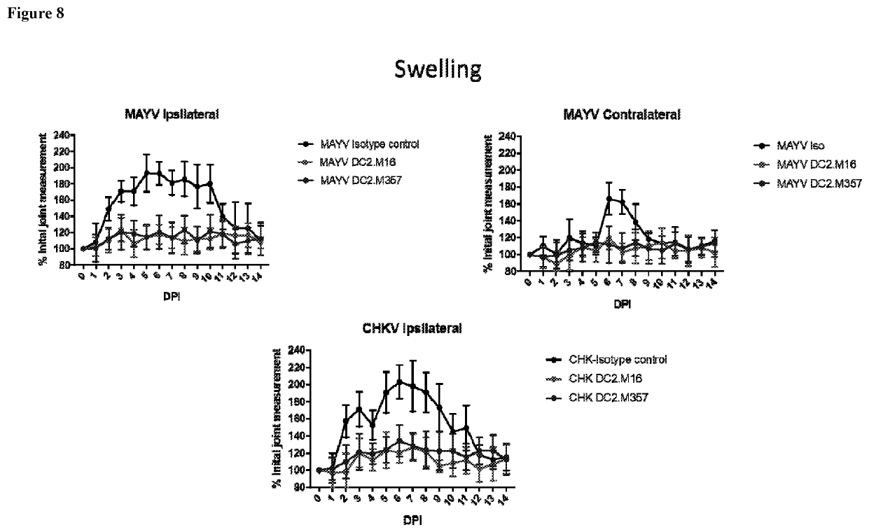

The cross-protective potential of mAbs DC2.M16 and DC2.M357 was investigated using joint swelling and viremia models for Chikungunya virus (La Reunion OPY1) and Mayaro virus (BeH407). MAbs were provided to 4-week old mice IP (100 μg/mouse) one day prior to inoculation via food pad injection of CHIKV or MAYV. DC2.M16- and DC2.M357-treated mice exhibited significantly reduced swelling of ipsilateral and contralateral joints in comparison to mice treated with isotype control (Iso) when challenged with MAYV (FIG. 8, top). Similarly, ipsilateral joint swelling was reduced upon CHIKV challenge relative to Isotype control (FIG. 8, bottom). These results indicate that mAbs DC2.M16 and DC2.M357 afford protection against CHIKV- and MAYV-induced arthritic disease.

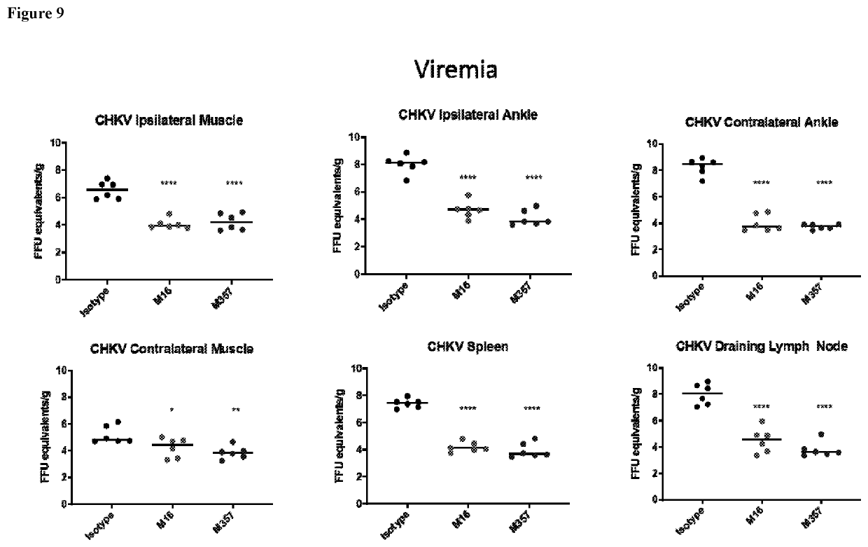

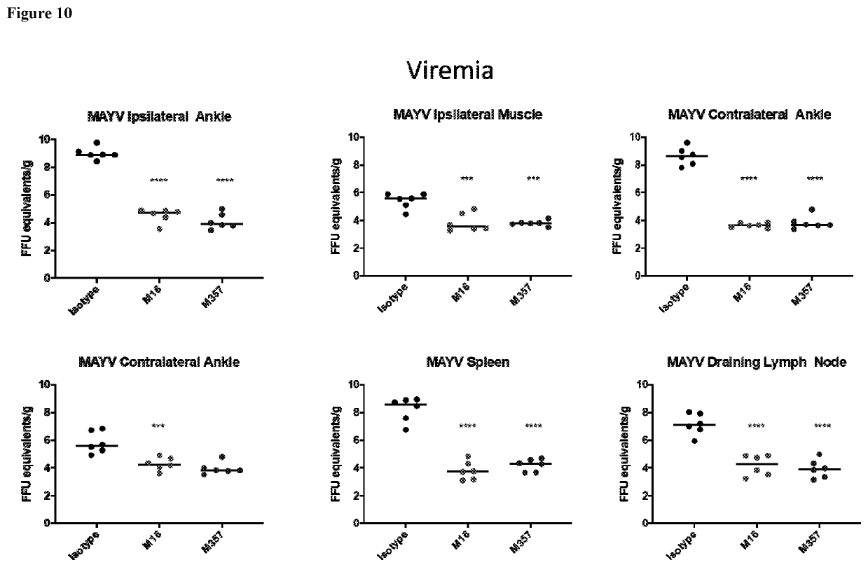

To further explore the protective potential against infection, viremia from the ipsilateral and contralateral tissues, as well as the spleen and lymph nodes was quantified for mAb-treated animals against CHIKV (FIG. 9) and MAYV (FIG. 10). In all cases, there was significantly lower viremia in all tissues for DC2.M16- and DC2.M357-treated mice than isotype control-treated mice, thus further demonstrating protective properties of these mAbs.

Although human bNAbs have been identified and extensively characterized in the context of HIV (41), influenza (42), ebolavirus (43, 44), and other pathogens, few have been described for alphaviruses. Murine CHK-265 and RRV-12, a human mAb recently isolated from a RRV convalescent donor, are among the only alphavirus bNAbs that have demonstrated cross-protection against multiple alphaviruses. Here, a single B cell sorting strategy using a heterologous MAYV antigen to capture and profile cross-reactive human mAbs from a CHIKV convalescent donor was employed, with the goal of identifying cross-protective mAbs. Serological studies of CHIKV patients have recently demonstrated cross-reactivity and cross-neutralization to heterologous alphaviruses, suggesting that cross-reactive and/or broadly-neutralizing mAbs may be elicited by natural CHIKV infection. 33 human mAbs that strongly reacted with both CHIKV and MAYV p62-E1 were isolated. These cross-reactive mAbs had diverse sequences and targeted both the E1 and E2 subunits of the alphavirus glycoprotein. Both neutralizing and non-neutralizing CHIKV-specific human mAbs targeting E1 were previously characterized (25). The current study identifies E1 as well as E2 as a target of human cross-reactive mAbs, although E1-specific mAbs were non-neutralizing.

Five human bNAbs that inhibited CHIKV and MAYV with IC₅₀ values ranging from 0.5-4.4 nM (75-660 ng/mL) were identified. The epitopes engaged by these mAbs map to the B domain of E2, which previously was identified as the target of murine bNAb CHK-265 and more recently, the human bNAb RRV-12. While all the DC2 bNAbs isolated competed with CHK-265, they exhibited differential neutralizing breadth and potency with respect to heterologous viruses ONNV, RRV, and SFV. The viral escape mutant studies revealed that human bNAbs engage related but distinct regions of the B domain of E2 and that fine recognition specificity is a determinant of neutralization breadth. BNAbs that mapped to G209 and K215 epitope were more limited in breadth than DC2.M357, which targeted K189 and N218. The DC2.M357 epitope appears highly related to that of murine CHK-265, which shares similar escape mutations and neutralization profiles. RRV-12, the only reported human bNAb to protect against multiple alphaviruses, was isolated from an RRV convalescent donor and also shares similar features with CHK-265. Although three-dimensional structures of DC2.M108 and DC2.M357 bound to E2 are not yet available, the viral escape mutations suggest different angles of mAb engagement. Given the high density of viral spikes on the alphavirus particle, this might in part explain their differences in potency and breadth, as mAb binding to the lateral side of the B domain by DC2.M108 would be predicted to be more sterically hindered by adjacent spikes. Thus, the work identifies distinct classes of human bNAbs that map to different regions on the E2 B domain, which may be important to consider in the design of alphavirus vaccines with broad reactivity. Recent work has demonstrated that CHIKV infection or vaccination with a vaccinia-based CHIKV vaccine confers variable and limited cross-protection to heterologous alphaviruses (45). Another study demonstrated that CHIKV and MAYV adenoviral vector-based vaccines only partially protected against heterologous viral challenge in mice (46). Designed immunogens guided by human bNAb epitopes may aid the targeted elicitation of more robust, cross-protective antibody responses.

Sequence analysis of DC2 bNAbs revealed two distinct V-gene lineages. Four of the five human bNAbs (DC2.M16, DC2.M108, DC2.M131, and DC2.M230) were related clonally and shared IGHV3-11 and IGKV1-NL1 germline pairing, whereas DC2.M357 was distinct and utilized IGHV3-30 and IGKV3-11. Recently, IGHV3-11 usage was found to be prevalent in germline neutralizing mAbs that target the RSV F protein (47). Although the cross-reactive mAbs showed a range of somatic mutation, bNAbs were generally close to the germline V-gene sequence with very few substitutions. Moreover, analysis of DC2.M16 and DC2.M108 inferred germline mAbs showed equivalent binding and neutralization profiles compared to the wild-type somatically mutated antibody. Germline-like neutralizing antibodies have been recently identified for RSV (47), H7N9 Influenza virus (48), Zika virus (49, 50), Dengue virus (51), and SARS-CoV-2 (52). Such antibodies may be elicited early in the course of infection compared to those that undergo extensive affinity maturation and somatic mutation. Therefore, induction of broadly neutralizing alphavirus antibodies may not require sustained antigen production or exposure. Furthermore, since both IGHV3-11 germline-reverted and mature alphavirus bNAbs can neutralize multiple alphaviruses, strategies to preferentially stimulate this germline could potentially result in elicitation of higher levels of broadly neutralizing antibody responses. Recent work towards the development of germline-targeting vaccines that induce broadly-protective antibody responses has been described in HIV (53-55) and influenza (56, 57). Thus, targeted elicitation of bNAbs against alphaviruses from specific germlines may be advantageous in developing a broadly effective vaccine.

Currently, no antiviral treatments for arthritogenic alphaviruses exist, and therapeutic mAbs have shown promise in mice and non-human primates against homologous viruses, but less so against heterologous viruses. It was shown that two bNAbs (DC2.M16 and DC2.M357) confer protection in mouse models of both CHIKV and MAYV infection. Both mAbs markedly reduced alphavirus-induced joint swelling and viral infection and dissemination to a similar extent, despite having moderate (sub-nanomolar to low nanomolar range) neutralizing activity and having a 10-fold difference in potency. Recent work has demonstrated that in addition to neutralizing potency, Fc effector functions are important in mediating protection against alphaviruses (30, 58, 59). CHK-265 was shown to protect against heterologous RRV challenge despite moderate cross-neutralizing potency (37). The study, together with these findings, demonstrates the utility of evaluating in vivo properties of moderately inhibitory mAbs, as neutralizing potency may not be the only important correlate of protection. Notably, mAbs all contain a human IgG1 Fc that binds mouse FcγRI, FcγRIII, and FcγRIV, which may be important for effector functions and in vivo efficacy (60).

MAbs with fewer somatic mutations generally exhibit more favorable “drug-like” properties such as better conformational stability and reduced hydrophobicity and poly-reactivity (61). A broad antiviral antibody against arthritogenic alphaviruses may be useful due their similar (almost indistinguishable) clinical manifestations and continued global spread. The present study identifies candidate mAbs for further development as broad immunotherapies to combat alphavirus disease and provides insight into the potential for elicitation of similar broad responses in humans.

### REFERENCES

1. Holmes A C, Basore K, Fremont D H, & Diamond M S (2020) A molecular understanding of alphavirus entry. PLOS Pathogens 16(10):e1008876.
2. Levi L I & Vignuzzi M (2019) Arthritogenic Alphaviruses: A Worldwide Emerging Threat? Microorganisms 7(5):133.
3. Zaid A, et al. (Arthritogenic alphaviruses: epidemiological and clinical perspective on emerging arboviruses. The Lancet Infectious Diseases.
4. Chen W, et al. (2015) Arthritogenic alphaviruses: new insights into arthritis and bone pathology. Trends in Microbiology 23(1):35-43.
5. Suhrbier A, Jaffar-Bandjee M C, & Gasque P (2012) Arthritogenic alphaviruses—an overview. Nat Rev Rheumatol 8(7):420-429.
6. Crosby L, et al. (2016) Severe manifestations of chikungunya virus in critically ill patients during the 2013-2014 Caribbean outbreak. International Journal of Infectious Diseases 48:78-80.
7. Thiberville S-D, et al. (2013) Chikungunya fever: epidemiology, clinical syndrome, pathogenesis and therapy. Antiviral Res 99(3):345-370.
8. Yergolkar P N, et al. (2006) Chikungunya outbreaks caused by African genotype, India. Emerg Infect Dis 12(10):1580-1583.
9. Wahid B, Ali A, Rafique S, & Idrees M (2017) Global expansion of chikungunya virus: mapping the 64-year history. Int J Infect Dis 58:69-76.
10. Yactayo S, Staples J E, Millot V, Cibrelus L, & Ramon-Pardo P (2016) Epidemiology of Chikungunya in the Americas. The Journal of Infectious Diseases 214(suppl_5):S441-S445.
11. Diagne C T, et al. (2020) Mayaro Virus Pathogenesis and Transmission Mechanisms. Pathogens 9(9):738.
12. Hozé N, et al. (2020) Reconstructing Mayaro virus circulation in French Guiana shows frequent spillovers. Nat Commun 11(1):2842.
13. Long K C, et al. (2011) Experimental transmission of Mayaro virus by Aedes aegypti. Am J Trop Med Hyg 85(4):750-757.
14. Pereira T N, Rocha M N, Sucupira P H F, Carvalho F D, & Moreira L A (2018) Wolbachia significantly impacts the vector competence of Aedes aegypti for Mayaro virus. Scientific Reports 8(1):6889.
15. Pereira T N, Carvalho F D, De Mendonça S F, Rocha M N, & Moreira L A (2020) Vector competence of Aedes aegypti, Aedes albopictus, and Culex quinquefasciatus mosquitoes for Mayaro virus. PLOS Negl Trop Dis 14(4):e0007518.
16. Brustolin M, Pujhari S, Henderson C A, & Rasgon J L (2018) Anopheles mosquitoes may drive invasion and transmission of Mayaro virus across geographically diverse regions. PLOS Negl Trop Dis 12(11):e0006895.
17. Alto B W, Civana A, Wiggins K, Eastmond B, & Shin D (2020) Effect of Oral Infection of Mayaro Virus on Fitness Correlates and Expression of Immune Related Genes in Aedes aegypti. Viruses 12(7).
18. Kraemer M U G, et al. (2015) The global distribution of the arbovirus vectors Aedes aegypti and Ae. albopictus. eLife 4:e08347.
19. Acosta-Ampudia Y, et al. (2018) Mayaro: an emerging viral threat? Emerg Microbes Infect 7(1):163-163.
20. Jose J, Snyder J E, & Kuhn R J (2009) A structural and functional perspective of alphavirus replication and assembly. Future Microbiology 4(7):837-856.
21. Martín C S-S, Liu C Y, & Kielian M (2009) Dealing with low pH: entry and exit of alphaviruses and flaviviruses. Trends in Microbiology 17(11):514-521.
22. Vaney M C, Duquerroy S, & Rey F A (2013) Alphavirus structure: activation for entry at the target cell surface. Curr Opin Virol 3(2):151-158.
23. Voss J E, et al. (2010) Glycoprotein organization of Chikungunya virus particles revealed by X-ray crystallography. Nature 468(7324):709-712.
24. Uchime O, Fields W, & Kielian M (2013) The Role of E3 in pH Protection during Alphavirus Assembly and Exit. Journal of Virology 87(18):10255-10262.
25. Quiroz J A, et al. (2019) Human monoclonal antibodies against chikungunya virus target multiple distinct epitopes in the E1 and E2 glycoproteins. PLOS Pathogens 15(11):e1008061.
26. Pal P, et al. (2013) Development of a highly protective combination monoclonal antibody therapy against Chikungunya virus. PLoS pathogens 9(4):e1003312-e1003312.
27. Smith Scott A, et al. (2015) Isolation and Characterization of Broad and Ultrapotent Human Monoclonal Antibodies with Therapeutic Activity against Chikungunya Virus. Cell Host & Microbe 18(1):86-95.
28. Powell L A, et al. (2020) Human monoclonal antibodies against Ross River virus target epitopes within the E2 protein and protect against disease. PLoS pathogens 16(5):e1008517-e1008517.
29. Selvarajah S, et al. (2013) A neutralizing monoclonal antibody targeting the acid-sensitive region in chikungunya virus E2 protects from disease. PLoS Negl Trop Dis 7(9):e2423.
30. Earnest J T, et al. (2019) Neutralizing antibodies against Mayaro virus require Fc effector functions for protective activity. Journal of Experimental Medicine 216(10):2282-2301.
31. Zhou Q F, et al. (2020) Structural basis of Chikungunya virus inhibition by monoclonal antibodies. Proceedings of the National Academy of Sciences 117(44):27637.
32. Basore K, et al. (2019) Cryo-EM Structure of Chikungunya Virus in Complex with the Mxra8 Receptor. Cell 177(7):1725-1737.e1716.
33. Powell L A, et al. (2020) Human mAbs Broadly Protect against Arthritogenic Alphaviruses by Recognizing Conserved Elements of the Mxra8 Receptor-Binding Site. Cell Host & Microbe.
34. Martins K A, et al. (2019) Neutralizing Antibodies from Convalescent Chikungunya Virus Patients Can Cross-Neutralize Mayaro and Una Viruses. The American Journal of Tropical Medicine and Hygiene 100(6):1541-1544.
35. Webb E M, et al. (2019) Effects of Chikungunya virus immunity on Mayaro virus disease and epidemic potential. Scientific Reports 9(1):20399.
36. Fischer C, et al. (2020) Robustness of Serologic Investigations for Chikungunya and Mayaro Viruses following Coemergence. mSphere 5(1):e00915-00919.
37. Fox J M, et al. (2020) A cross-reactive antibody protects against Ross River virus musculoskeletal disease despite rapid neutralization escape in mice. PLOS Pathogens 16(8):e1008743.
38. Fox J M, et al. (2015) Broadly Neutralizing Alphavirus Antibodies Bind an Epitope on E2 and Inhibit Entry and Egress. Cell 163(5):1095-1107.
39. Townsend C L, et al. (2016) Significant Differences in Physicochemical Properties of Human Immunoglobulin Kappa and Lambda CDR3 Regions. Front Immunol 7:388.
40. Pierson T C & Diamond M S (2015) Chapter Five—A Game of Numbers: The Stoichiometry of Antibody-Mediated Neutralization of Flavivirus Infection. Progress in Molecular Biology and Translational Science, ed Klasse PJ (Academic Press), Vol 129, pp 141-166.
41. Burton D R & Hangartner L (2016) Broadly Neutralizing Antibodies to HIV and Their Role in Vaccine Design. Annual Review of Immunology 34(1):635-659.
42. Laursen N S & Wilson I A (2013) Broadly neutralizing antibodies against influenza viruses. Antiviral Res 98(3):476-483.
43. Bornholdt Z A, et al. (2019) A Two-Antibody Pan-Ebolavirus Cocktail Confers Broad Therapeutic Protection in Ferrets and Nonhuman Primates. Cell Host & Microbe 25(1):49-58.e45.
44. Wec A Z, et al. (2019) Development of a Human Antibody Cocktail that Deploys Multiple Functions to Confer Pan-Ebolavirus Protection. Cell Host & Microbe 25(1):39-48.e35.
45. Nguyen W, et al. (2020) Arthritogenic Alphavirus Vaccines: Serogrouping Versus Cross-Protection in Mouse Models. Vaccines (Basel) 8(2):209.
46. Kroon Campos R, et al. (2020) Adenoviral-Vectored Mayaro and Chikungunya Virus Vaccine Candidates Afford Partial Cross-Protection From Lethal Challenge in A129 Mouse Model. Front Immunol 11:591885.
47. Goodwin E, et al. (2018) Infants Infected with Respiratory Syncytial Virus Generate Potent Neutralizing Antibodies that Lack Somatic Hypermutation. Immunity 48(2):339-349.e335.
48. Yu F, et al. (2017) A Potent Germline-like Human Monoclonal Antibody Targets a pH-Sensitive Epitope on H7N9 Influenza Hemagglutinin. Cell Host & Microbe 22(4):471-483.e475.
49. Gao F, et al. (2019) Development of a Potent and Protective Germline-Like Antibody Lineage Against Zika Virus in a Convalescent Human. Front Immunol 10:2424-2424.
50. Magnani D M, et al. (2017) A human inferred germline antibody binds to an immunodominant epitope and neutralizes Zika virus. PLOS Neglected Tropical Diseases 11(6):e0005655.
51. Hu D, et al. (2019) A broadly neutralizing germline-like human monoclonal antibody against dengue virus envelope domain III. PLoS pathogens 15(6):e1007836-e1007836.
52. Kreer C, et al. (2020) Longitudinal Isolation of Potent Near-Germline SARS-CoV-2-Neutralizing Antibodies from COVID-19 Patients. Cell 182(4):843-854.e812.
53. Ward A B & Wilson I A (2020) Innovations in structure-based antigen design and immune monitoring for next generation vaccines. Current opinion in immunology 65:50-56.
54. Steichen J M, et al. (2019) A generalized HIV vaccine design strategy for priming of broadly neutralizing antibody responses. Science (New York, N.Y.) 366(6470).
55. Jardine J, et al. (2013) Rational HIV immunogen design to target specific germline B cell receptors. Science (New York, N.Y.) 340(6133):711-716.
56. Cheung C S-F, et al. (2020) Identification and Structure of a Multidonor Class of Head-Directed Influenza-Neutralizing Antibodies Reveal the Mechanism for Its Recurrent Elicitation. Cell Reports 32(9):108088.
57. Sangesland M, et al. (2019) Germline-Encoded Affinity for Cognate Antigen Enables Vaccine Amplification of a Human Broadly Neutralizing Response against Influenza Virus. Immunity 51(4):735-749.e738.
58. Earnest J T, et al. (2021) The mechanistic basis of protection by non-neutralizing anti-alphavirus antibodies. Cell Rep 35(1):108962.
59. Fox J M, et al. (2019) Optimal therapeutic activity of monoclonal antibodies against chikungunya virus requires Fc-FcγR interaction on monocytes. Science Immunology 4(32):eaav5062.
60. Dekkers G, et al. (2017) Affinity of human IgG subclasses to mouse Fc gamma receptors. mAbs 9(5):767-773.
61. Shehata L, et al. (2019) Affinity Maturation Enhances Antibody Specificity but Compromises Conformational Stability. Cell Reports 28(13):3300-3308.e3304.
62. Brault A C, et al. (2004) Infection patterns of o'nyong nyong virus in the malaria-transmitting mosquito, Anopheles gambiae. Insect Mol Biol 13(6):625-635.
63. Kuhn R J, Niesters H G M, Hong Z, & Strauss J H (1991) Infectious RNA transcripts from ross river virus cDNA clones and the construction and characterization of defined chimeras with sindbis virus. Virology 182(2):430-441.
64. Chuong C, Bates T A, & Weger-Lucarelli J (2019) Infectious cDNA clones of two strains of Mayaro virus for studies on viral pathogenesis and vaccine development. Virology 535:227-231.
65. Liljeström P, Lusa S, Huylebroeck D, & Garoff H (1991) In vitro mutagenesis of a full-length cDNA clone of Semliki Forest virus: the small 6,000-molecular-weight membrane protein modulates virus release. Journal of virology 65(8):4107-4113.
66. Hardwick J M & Levine B (2000) Sindbis virus vector system for functional analysis of apoptosis regulators. Methods Enzymol 322:492-508.
67. Sánchez-San Martín C, Nanda S, Zheng Y, Fields W, & Kielian M (2013) Cross-inhibition of chikungunya virus fusion and infection by alphavirus E1 domain III proteins. J Virol 87(13):7680-7687.
68. Tiller T, et al. (2008) Efficient generation of monoclonal antibodies from single human B cells by single cell RT-PCR and expression vector cloning. J Immunol Methods 329(1-2):112-124.
69. Mazor Y, Barnea I, Keydar I, & Benhar I (2007) Antibody internalization studied using a novel IgG binding toxin fusion. J Immunol Methods 321(1-2):41-59.
70. Brochet X, Lefranc M P, & Giudicelli V (2008) IMGT/V-QUEST: the highly customized and integrated system for IG and TR standardized V-J and V-D-J sequence analysis. Nucleic Acids Res 36(Web Server issue):W503-508.

1. An anti-alphavirus antibody or alphavirus-binding fragment thereof, wherein said antibody or fragment thereof comprises:
(1) a heavy chain comprising the CDRS set forth in GFTFSNYA (SEQ ID NO:1), ISFDGSIN (SEQ ID NO: 2), CARDRYYYDSSAYFLIDAFDI (SEQ ID NO:3); and
(2) a light chain comprising the CDRS set forth in QSVSSH (SEQ ID NO:4), DAS (SEQ ID NO:5), and QQRSNWPPIT (SEQ ID NO:6).

2. An anti-alphavirus antibody or alphavirus-binding fragment thereof, wherein said antibody or fragment thereof comprises:
(1) a heavy chain variable domain set forth in SEQ ID NO:199; and
(2) a light chain variable domain set forth in SEQ ID NO:200.

3. The antibody, or alphavirus-binding fragment thereof, of claim 1, comprising a non-naturally occurring Fc region.

4. The antibody, or alphavirus-binding fragment thereof, of claim 1, comprising a mutated human Fc region.

5. The antibody, or alphavirus-binding fragment thereof, of claim 1, which is an Immunoglobulin G type antibody.

6. The antibody, or alphavirus-binding fragment thereof, of claim 1, wherein the antibody, or alphavirus-binding fragment thereof, binds an alphavirus with a binding affinity (K_{D}) of from about 0.005 nM to 100 nM.

7. The antibody, or alphavirus-binding fragment thereof, of claim 1, which is a monoclonal antibody.

8. The antibody, or alphavirus-binding fragment thereof, of claim 1, which is a recombinant antibody.

9. The alphavirus-binding fragment of claim 1, which is an Fab, F(ab)2 or scFv.

10. The antibody, or alphavirus-binding fragment thereof, of claim 1, wherein the antibody, or alphavirus-binding fragment thereof binds to E2.

11. The antibody, or alphavirus-binding fragment thereof, of claim 1, wherein the antibody, or alphavirus-binding fragment thereof binds to B domain of E2.

12. The antibody, or alphavirus-binding fragment thereof, of claim 1, wherein the alphavirus is selected from the group consisting of Chikungunya virus (CHIKV), Mayaro virus (MAYV), Ross River virus (RRV), O'nyong-nyong virus (ONNV), and Semliki Forest virus (SFV).

13. A method for treating an alphavirus infection in a subject, comprising administering an antibody or antigen-binding fragment thereof of claim 1 in an amount effective to treat the alphavirus infection in the subject.

14. A method for inhibiting an alphavirus infection in a subject, comprising administering an antibody or antigen-binding fragment thereof of claim 1 in an amount effective to inhibit the alphavirus infection in the subject.

15-23. (canceled)

24. An isolated nucleic acid molecule encoding the antibody, or antigen-binding fragment thereof, of claim 1.

25. A vector comprising the nucleic acid molecule of claim 24.

26. A host cell comprising the nucleic acid molecule of claim 24.

27. A method of producing an anti-alphavirus antibody comprising culturing the host cell of claim 26, under conditions wherein the anti-alphavirus antibody is produced by the host cell.

28. A pharmaceutical composition comprising an anti-alphavirus antibody, or alphavirus-binding fragment thereof, of claim 1, and a pharmaceutically acceptable excipient.

29. A method of reducing an activity of an alphavirus in a subject in need thereof, comprising administering to said subject a therapeutically effective amount of the anti-alphavirus antibody, or alphavirus-binding fragment thereof, of claim 1.

30. A method of treating a disease, disorder, or condition mediated by, or related to increased activity of an alphavirus in a subject a therapeutically effective amount of the anti-alphavirus antibody, or alphavirus-binding fragment thereof, of claim 1.

31. An assay device for selectively detecting an alphavirus in a biological sample comprising:
a first portion comprising a first plurality of anti-alphavirus antibodies of claim 1,
wherein the antibodies are each attached to their own reporting entity;
a second portion comprising a second plurality of anti-alphavirus antibodies.

32-41. (canceled)
